(19) United States
(12) Patent Application Publication
(10) Pub. No.: US 2020/0216906 A1
(43) Pub. Date: Jul. 9, 2020

(54) METHODS AND COMPOSITIONS RELATING TO THE DIAGNOSIS AND TREATMENT OF CANCER

(71) Applicant: PRESIDENT AND FELLOWS OF HARVARD COLLEGE, Cambridge, MA (US)

(72) Inventors: Taran GUJRAL, Brookline, MA (US); Marc W. KIRSCHNER, Newton, MA (US)

(73) Assignee: PRESIDENT AND FELLOWS OF HARVARD COLLEGE, Cambridge, MA (US)

(21) Appl. No.: 15/754,695

(22) PCT Filed: Aug. 23, 2016
(86) PCT No.: PCT/US2016/048133

§ 371 Date: Feb. 23, 2018

Related U.S. Application Data
(60) Provisional application No. 62/209,682, filed on Aug. 25, 2015.

(51) Int. Cl. C12Q 1/6886

(52) U.S. Cl. CPC C12Q 1/6886; C12Q 2600/106; C12Q 2600/158; C12Q 2600/156

(57) ABSTRACT

Described herein are methods and compositions relating to the treatment of cancer, e.g., methods which account for a subject's Hippo pathway activity/mutational status or which relate to combination treatments that influence the subject's Hippo pathway activity in order to enhance the effectiveness of chemotherapeutics.

### CROSS-REFERENCE TO RELATED APPLICATIONS

This application is a 35 U.S.C. § 371 National Phase Entry Application of International Application No. PCT/US2016/048133 filed Aug. 23, 2016, which designates the U.S. and claims priority under 35 U.S.C. § 119(e) to U.S. Provisional Patent Application Ser. No. 62/209,682, filed Aug. 25, 2015, the contents of which are incorporated herein by reference in their entireties.

### GOVERNMENT SUPPORT

This invention was made with government support under Grant Nos. ROI 152189 and R01 HD073104 awarded by the National Institutes of Health. The U.S. government has certain rights in the invention.

### SEQUENCE LISTING

The instant application contains a Sequence Listing which has been submitted electronically in ASCII format and is hereby incorporated by reference in its entirety. Said ASCII copy, created on Aug. 22, 2016, is named 002806-085541-PCT_SL.txt and is 134,539 bytes in size.

### TECHNICAL FIELD

The technology described herein relates to methods of diagnosing, prognosing, and treating cancer.

### BACKGROUND

Pancreatic ductal adenocarcinoma (PDAC) is one of the most lethal forms of cancer. The 1- and 5-year survival rates for PDAC are about 10% and 4.6%, respectively, which are the lowest survival rates of all major cancers. Currently, the nucleoside analogue gemcitabine is the first line treatment of locally advanced and metastatic pancreatic cancer. However, most patients (>75%) treated with gemcitabine do not have an objective response to treatment and only a minority obtains stabilization of disease or partial response.

### SUMMARY

As described herein, the inventors have discovered that cancer cells develop resistance to certain chemotherapeutics (e.g. gemcitabine) as the cell density increases. This developed resistance is controlled by alterations in the Hippo-YAP signaling pathway. The sensitivity of the cells to the chemotherapeutics can be restored by suppressing the Hippo-YAP pathway. This discovery permits both improved methods of treatment by 1) administering gemcitabine only to subjects who are sensitive to it, and 2) by inducing gemcitabine sensitivity by administering Hippo-YAP signaling inhibitors.

In one aspect, described herein is a method of treating cancer, the method comprising administering a chemotherapeutic selected from the group consisting of: an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; to a subject having cancer cells determined to have:
a. a deletion, a truncation or inactivating mutation in FAT4; LATS1; LATS2; STK11; or NF2;
b. decreased expression of FAT4; LATS1; LATS2; STK11; or NF2 relative to a reference;
c. increased expression of YAP; CTGF; AREG; AMOTL2; AXL; or BIRC5 relative to a reference;
d. decreased phosphorylation of YAP relative to a reference; or
e. increased nuclear localization of YAP relative to a reference.

In one aspect, provided herein is a therapeutically effective amount of a chemotherapeutic selected from the group consisting of: an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; for use in a method of treating cancer, the method comprising administering the cytotoxic chemotherapeutic to a subject having cancer cells determined to have:
a. a deletion, a truncation or inactivating mutation in FAT4; LATS1; LATS2; STK11; or NF2;
b. decreased expression of FAT4; LATS1; LATS2; STK11; or NF2 relative to a reference;
c. increased expression of YAP; CTGF; AREG; AMOTL2; AXL; or BIRC5 relative to a reference;
d. decreased phosphorylation of YAP relative to a reference; or
e. increased nuclear localization of YAP relative to a reference.

In some embodiments, the antimetabolite or nucleoside analog is selected from the group consisting of: gemcitabine; 5-FU; cladribine; cytarabine; tioguanine; mercaptopurine; and clofarabine. In some embodiments, the antifolate is methotrexate. In some embodiments, the topoisomerase I inhibitor is camptothecin, topotecan, or irrenotecan. In some embodiments, the topoisomerase II inhibitor is selected from the group consisting of: epirubicin; daunorubicin; doxorubicin; valrubicin; teniposide; etopiside; and mitoxantrone. In some embodiments the anthracycline is selected from the group consisting of: epirubicin; daunorubicin; doxorubicin; and valrubicin. In some embodiments, the tubulin modulator is ixabepilone. In some embodiments, the Src family kinase inhibitor or BCR-Abl kinase inhibitor is imatinib. In some embodiments, the DNA cross-linking agent is mitomycin.

In one aspect, provided herein is a method of treating cancer, the method comprising administering a chemotherapeutic selected from the group consisting of: an antimetabolite; an anthracylcine; an anthracycline topoisomerase II inhibitor; a proteasome inhibitor; an mTOR inhibitor; an RNA synthesis inhibitor; a peptide synthesis inhibitor; an alkylating agent; an antiandrogen; a Src family kinase inhibitor; a BCR-Abl kinase inhibitor; a MEK inhibitor; and a kinase inhibitor; to a subject having cancer cells determined not to have:
a. a deletion, a truncation, or inactivating mutation in FAT4; LATS1; LATS2; STK11; or NF2;
b. decreased expression of FAT4; LATS1; LATS2; STK11; or NF2 relative to a reference;
c. increased expression of YAP; CTGF; AREG; AMOTL2; AXL; or BIRC5 relative to a reference;
d. decreased phosphorylation of YAP relative to a reference; or
e. increased nuclear localization of YAP relative to a reference.

In one aspect, provided herein is a therapeutically effective amount of a compound selected from the group consisting of: an antimetabolite; an anthracylcine; an anthracycline topoisomerase II inhibitor; a proteasome inhibitor; an mTOR inhibitor; an RNA synthesis inhibitor; a peptide synthesis inhibitor; an alkylating agent; an antiandrogen; a Src family kinase inhibitor; a BCR-Abl kinase inhibitor; a MEK inhibitor; and a kinase inhibitor; for use in a method of treating cancer, the method comprising administering the compound to a subject having cancer cells determined not to have:
a. a deletion, a truncation, or inactivating mutation in FAT4; LATS1; LATS2; STK11; or NF2;
b. decreased expression of FAT4; LATS1; LATS2; STK11; or NF2 relative to a reference;
c. increased expression of YAP; CTGF; AREG; AMOTL2; AXL; or BIRC5 relative to a reference;
d. decreased phosphorylation of YAP relative to a reference; or
e. increased nuclear localization of YAP relative to a reference.

In some embodiments, the anthracycline toposisomerase II inhibitor is selected from the group consisting of: daunorubicin; doxorubicin; epirubicin; and valrubicin. In some embodiments, the anthracycline is selected from the group consisting of: daunorubicin; doxorubicin; epirubicin; and valrubicin. In some embodiments, the proteasome inhibitor is carfilzomib or bortezomib. In some embodiments, the mTOR inhibitor is everolimus. In some embodiments the RNA synthesis inhibitor is triethylenemelamine, dactinomycin, or plicamycin. In some embodiments, the kinase inhibitor is ponatinib or trametinib. In some embodiments, the Src family kinase inhibitor or BCR-Abl kinase inhibitor is ponatinib. In some embodiments, the MEK inhibitor is trametinib. In some embodiments, the antiandrogen is enzalutamide. In some embodiments. the peptide synthesis inhibitor is omacetaxine mepesuccinate.

In some embodiments of any of the aspects described herein, the mutation in FAT4; LATS1; LATS2; STK11; or NF2 is selected from Table 2. In some embodiments of any of the aspects described herein, the method further comprises a step of detecting the presence of one or more of:
a. a deletion, a truncation, or inactivating mutation in FAT4; LATS1; LATS2; STK11; or NF2;
b. decreased expression of FAT4; LATS1; LATS2; STK11; or NF2 relative to a reference;

c. increased expression of YAP; CTGF; AREG; AMOTL2; AXL; or BIRC5 relative to a reference;
d. decreased phosphorylation of YAP relative to a reference; or
e. increased nuclear localization of YAP relative to a reference.

In one aspect, provided herein is a method of treating cancer, the method comprising administering
a. a chemotherapeutic selected from the group consisting of:
   an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; and
b. an inhibitor of FAT4; STK11; LATS1; LATS2; or NF2; or an agonist of YAP.

In one aspect, provided herein is a therapeutically effective amount of a chemotherapeutic selected from the group consisting of: an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; and a therapeutically effective amount of an inhibitor of FAT4, STK11, LATS1, LATS2, or NF2, or an agonist of YAP; for use in a method of treating cancer, the method comprising administering i) the chemotherapeutic and ii) the inhibitor of FAT4, STK11, LATS1, LATS2, or NF2, or agonist of YAP; to a subject in need of treatment for cancer. In some embodiments, the antimetabolite or nucleoside analog is selected from the group consisting of: gemcitabine; 5-FU; cladribine; cytarabine; tioguanine; mercaptopurine; and clofarabine. In some embodiments, the antifolate is methotrexate. In some embodiments, the topoisomerase I inhibitor is camptothecin, topotecan, or irrenotecan. In some embodiments, the topoisomerase II inhibitor is selected from the group consisting of: epirubicin; daunorubicin; doxorubicin; valrubicin; teniposide; etopiside; and mitoxantrone. In some embodiments the anthracycline is selected from the group consisting of: epirubicin; daunorubicin; doxorubicin; and valrubicin. In some embodiments, the tubulin modulator is ixabepilone. In some embodiments, the Src family kinase inhibitor or BCR-Abl kinase inhibitor is imatinib. In some embodiments, the DNA cross-linking agent is mitomycin.

In some embodiments of any of the aspects described herein, the agonist of YAP is a non-phospho, active form of YAP (e.g. one or more of S61A, S109A, S127A, S128A, S131A, S163A, S164A, S381A mutants) or a nucleic acid encoding a non-phospho, active form of YAP. In some embodiments of any of the aspects described herein, the inhibitor of FAT4; STK11; LATS1; LATS2; or NF2 is an inhibitory nucleic acid. In some embodiments of any of the aspects described herein, the inhibitor of STK11 is AZ-23. In some embodiments of any of the aspects described herein, the inhibitor of LATS2 is GSK690693; AT7867; or PF-477736.

In some embodiments of any of the aspects described herein, the cancer is pancreatic cancer; pancreatic ductal adenocarcinoma; metastatic breast cancer; breast cancer; bladder cancer; small cell lung cancer; lung cancer; ovarian cancer; stomach cancer; uterine cancer; mesothelioma; adenoid cystic carcinoma; lymphoid neoplasm; kidney cancer; colorectal cancer; adenoid cystic carcinoma; prostate cancer; cervical cancer; head and neck cancer; and glioblastoma.

In one aspect, provided herein is an assay comprising: detecting, in a test sample obtained from a subject in need of treatment for cancer;
i. a deletion, a truncation or inactivating mutation in FAT4; LATS1; LATS2; STK11; or NF2;
ii. decreased expression of FAT4; LATS1; LATS2; STK11; or NF2 relative to a reference;
iii. increased expression of YAP; CTGF; AREG; AMOTL2; AXL; or BIRC5 relative to a reference;
iv. decreased phosphorylation of YAP relative to a reference; or
v. increased nuclear localization of YAP relative to a reference.
   wherein the presence of any of i.-v. indicates the subject is more likely to respond to treatment with a chemotherapeutic selected from the group consisting of: an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor. In some emboidments, the absence of i.-v. indicates the subject should receive treatment with a treatment selected from the group consisting of: an antimetabolite; an anthracylcine; an anthracycline topoisomerase II inhibitor; a proteasome inhibitor; an mTOR inhibitor; an RNA synthesis inhibitor; a peptide synthesis inhibitor; an alkylating agent; an antiandrogen; a Src family kinase inhibitor; a BCR-Abl kinase inhibitor; a MEK inhibitor; and a kinase inhibitor.

In some embodiments of any of the aspects described herein, the determining step comprises measuring the level of a nucleic acid. In some embodiments of any of the aspects described herein, the measuring the level of a nucleic acid comprises measuring the level of a RNA transcript. In some embodiments of any of the aspects described herein, the level of the nucleic acid is determined using a method selected from the group consisting of: RT-PCR; quantitative RT-PCR; Northern blot; microarray based expression analysis; next-generation sequencing; and RNA in situ hybridization. In some embodiments of any of the aspects described herein, the determining step comprises determining the sequence of a nucleic acid. In some embodiments of any of the aspects described herein, the determining step comprises measuring the level of a polypeptide. In some embodiments of any of the aspects described herein, the polypeptide level is measured using immunochemistry. In some embodiments of any of the aspects described herein, the immunochemistry comprises the use of an antibody reagent which is detectably labeled or generates a detectable signal. In some embodiments of any of the aspects described herein, the level of the polypeptide is determined using a method selected from the group consisting of: Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy; FACS; and immunoelectrophoresis assay. In some embodiments of any of the aspects described herein, the expression level is normalized relative to the expression level of one or more reference genes or reference proteins. In some embodiments of any of the aspects described herein, the reference level is the expression level in a prior sample obtained from the subject. In some embodiments of any of the aspects described herein, the sample comprises a biopsy; blood; serum; urine; or plasma.

### BRIEF DESCRIPTION OF THE DRAWINGS

FIG. 1 depicts a graph demonstrating that “switching-off” Hippo pathway confers sensitivity to gemcitabine in pancreatic cancer. Dose response curve of gemcitabine in Panc02.13 cells grown in 3D spheroid. Cells were either transfected with GFP vector (GFP), or active form of YAP (YAPS6A) or knockdown of NF2 (NF2sh).

FIG. 2 depicts graphs of a live-cell kinetic cell growth assay used to characterize the phenotypic effect of gemcitabine in a panel of pancreatic cancer cell lines. Plots depict the effect of gemcitabine on cell growth of five pancreatic cancer cell lines.

FIG. 3 depicts graphs of dose response curves of gemcitabine treated pancreatic cancer cell lines. The respective GC₅₀ for each cell line is also indicated.

FIG. 4 depicts plots demonstrating the effect of six cytotoxic drugs on growth of seven pancreatic cancer cell lines under sparse and dense conditions. The efficacy of gemcitabine, doxorubisin and camptothecin was density-dependent while the effects of paclitaxel, Docetaxel and Oxaliplatin were largely density independent.

FIG. 5 depicts a plot showing changes in protein levels or phosphorylation which occur in ASPC1 cells grown under low or high densities. Many growth factor signaling proteins such as Erk, Akt and S6 ribosomal proteins is downregulated when cells are grown in dense cultures. Increase in phosphorylation of YAP in density-dependent manner is also observed. The right panel depicts a western blot demonstrating an increase in phosphorylation of YAP in a density-dependent manner in Bxpc3 cells.

FIG. 6 depicts graphs demonstrating that suppressing Hippo pathway by expression of non-phospho, active form of YAP (YAPS6A) sensitizes pancreatic cancer cells to gemcitabine (left panel) and 5-FU (right panel). A plot showing the effect of gemcitabine on the growth of Panc02.13 cells expressing vector only or YapS6A construct grown at high cell density.

FIG. 7 depicts Western blots showing expression of YAPS6A sensitizes cells to gemcitabine and activates apoptosis. Pan02.13 cells expressing vector control or YAPS6A were treated with 50 nM Gemcitabine for 48 hours. Whole cell lysates were collected and subjected to western blotting. Apoptosis is measured by immunobloting with cleaved caspases 3/7 or PARP. Blots were also stained with anti-β-actin for loading control.

FIG. 8 depicts graphs demonstrating that suppressing Hippo pathway by expression of non-phospho, active form of YAP (YAPS6A) or knockdown of NF2 (upstream regulator of YAP phosphorylation) sensitizes pancreatic cancer cells to gemcitabine and 5-FU in 3D spheroid culture. Depicts are dose response curves of treated Panc02.13 cells expressing GFP vector, YAPS6A plasmid or NF2shRNA grown as 3D speheroid to the indicated compounds.

FIG. 9 depicts a graph demonstrating that activation of YAP decreases expression of several multidrug transporters. mRNA expression profiles comparing 84 drug transporters in Panc02.13 cells expressing vector control or YAPS6A. Expression of drug transporters which are significantly (p<0.05) are indicated in red while significantly upregulated transporters are indicated in green.

FIG. 10 depicts the density and YAP-dependent protein expression of several multidrug transporters. Left, Western blots demonstrating increase in protein expression of drug transporters ABCG2 and LRP with cell density. Right, Western blots demonstrating decrease in LRP protein expression upon overexpression of YAPS6A or NF2 knockdown.

FIG. 11 depicts plots demonstrating gemcitabine efflux (release in the medium) in Panc02.13 cells either grown at low/high densities (bottom left) or with overexpression of YAPS6A (bottom right). The top panel depicts the intracellular concentration of gemcitabine in Panc02.13 cells either grown at low/high densities.

FIG. 12 demonstrates that activation of YAP decreases expression of CDA (cytidine deaminase), the key enzyme that metabolizes the drug following its transport into the cell. Top, western blots showing protein expression of CDA in Panc02.13 cells expressing vector control, YAPS6A or NF2shRNA. Bottom, mRNA expression of CDA is significantly decreased in Panc02.13 cells expressing, YAPS6A or NF2shRNA compared with vector only control. The mRNA expression of dCK do not change with overexpression of YAPS6A or NF2shRNA.

FIG. 13 depicts a table of the percentage of various cancer types harboring mutations or deletions in the Hippo pathway genes. Data for this table was compiled using web-based cBioPortal for Cancer Genomics (http://cbioportal.org) [2].

FIG. 14 depicts a graph demonstrating that mesothelioma cells harboring LATS2 deletion are sensitive to gemcitabine and restoring LATS2 expression confers drug resistance. A plot showing the effect of gemcitabine on growth of H2052-mesothelioma cells in the presence or absence of LATS2 expression.

FIG. 15 depicts graphs demonstrating that low expression of NF2 gene signature is associated with prolong patient survival in pancreatic cancers. Kaplan-Meier curves of overall survival of pancreatic cancer patients with low or high levels of NF2 expression in two independent studies.

FIG. 16 depicts graphs demonstrating that responses of Aspc1 and Panc02.13 cells to gemcitabine are density-dependent.

FIG. 17 depicts graphs demonstrating that Yap activation sensitizes pancreatic cancer cells to cytotoxic drugs. 119 FDA-approved oncology drugs were tested in pancreatic cancer cells using 3D spheroid growth assays. Left, A plot showing most of the drugs are ineffective in Panc02.13 GFP expressing cells with EC50>1 μM. Some of the drugs which blocked spheroid growth in parental Panc02.13 cells are indicated. Right, YapS6A expressing Panc02.13 are sensitive to 15 additional drugs which includes antimetabolites, anthracyclines, topoisomerase inhibitors and kinase inhibitors (indicated in red).

FIG. 18 depicts graphs demonstrating that YAP activation (e.g. by use of YAPS6A) sensitizes Panc02 cells to antimetabolite drugs.

FIG. 19 depicts graphs demonstrating that YAP activation (e.g. by use of YAPS6A) sensitizes Panc02 cells to topoisomerase inhibitor drugs.

FIGS. 20A-20E demonstrate cell crowding-dependent response to gemcitabine in pancreatic cancer. FIG. 20A depicts aschematic showing live-cell kinetic cell growth assay used to characterize the phenotypic effect of gemcitabine in a panel of pancreatic cancer cell lines. Gemcitabine-mediated GC50 (50% inhibition in growth compared with control) for each cell line was calculated. FIG. 20B depicts a plot showing affect on gemcitabine on growth of 15 pancreatic cancer cell lines. Literature curated values of cell line specific GC50 are also indicated. FIG. 20C depicts graphs of crowding affects gemcitabine response. Plots show cell growth curves of Aspc1 (top) and Patu-8988S (bottom) cells grown in low or high crowding conditions. FIG. 20D depicts graph demonstrating that all cell lines were sensitive or resistant to gemcitabine in low or high crowding conditions respectively. FIG. 20E depicts graphs demonstrating that replating cells at low density restored sensitive to gemcitabine.

FIG. 21A-21C demonstrate that YAP activation sensitizes pancreatic cancer cells to cytotoxic drugs. FIG. 21A depicts proteomic changes in six pancreatic cancer cell lines grown in five different crowding conditions, performed using reverse phase protein arrays. Representative images show levels of phosho-S6, β-actin and GAPDH. FIG. 21B depicts Western blots showing expression of YAPS6A sensitizes cells to gemcitabine and activates apoptosis. Pan02.13 cells expressing vector control or YAPS6A were treated with 50 nM Gemcitabine for 48 hours. Whole cell lysates were collected and subjected to western blotting. Apoptosis was measured by immunobloting with cleaved caspases 3/7 or PARP. Blots were also stained with anti-β-actin for loading control. FIG. 21C depicts a schematic showing 3D-spheroid assay used for chemical screening. Cells were grown in round-bottom plates for two days to form spheroid of approximately 400 microns, followed by dose-dependent drug treatment and live cell imaging for 4 days. A dose response curve is then use to determine the effect of each drug on spheroid growth.

FIGS. 22A-22F demonstrate that Hippo-YAP pathway affects gemcitabine availability by modulating its efflux and metabolism. FIG. 22A depicts a plot showing increased gemcitabine efflux (release in the medium) in Panc02.13 cells either grown at low/high crowding conditions. Radioactive counts were normalized by total protein from each sample. FIG. 22B depicts graphs of gemcitabine and dFdU efflux in Panc02.13 cells expressing either vector control or YAPS6A measured using LC/MS. FIG. 22C depicts Western blots showing increase in protein expression of drug transporters ABCG2 and LRP with cell crowding. FIG. 22D depicts Western blots showing protein expression of CDA in Panc02.13.13 cells expressing vector control, YAPS6A or NF2shRNA. FIG. 22E demonstrates that protein levels of CDA change with cell crowding Western blots showing protein levels of CDA in three different pancreatic cancer cell lines. Blots were also stained with anti-β-actin for loading control. FIG. 22F demonstrates that Hippo-YAP pathway negatively regulates ABCG2 and CDA expression. ABCG2 and CDA expression levels were measured using promoter reporter construct in Panc02.13 cells expressing NF2shRNA or control siRNA. Data were normalized to internal control (SEAP) activity.

FIGS. 23A-23D demonstrate that Hippo pathway genetic aberrations confer sensitivity to gemcitabine in several cancer types. FIG. 23A depicts a plot showing dose-dependent effect of gemcitabine on growth of A549 cells (carrying STK11 mutation) in 3D-spheroid. FIG. 23B depicts a table summarizing the effect of gemcitabine on growth of six different cancer cell lines carrying Hippo pathway mutations. The relative GC50 and mutated or deleted Hippo pathway gene for each cell line is also listed. FIG. 23C demonstrates that ectopic expression of LATS2 increases the expression of ABCG2 and CDA in H2052 cells. FIG. 23D depicts plots showing relative levels of gemcitabine and dFdU effluxed from H2052 parental or H2052 expressing LATS2 cells.

FIGS. 24A-24D demonstrate that YAP activation sensitizes pancreatic tumors to gemcitabine in mouse xenograft models. FIGS. 24A-24B demonstrate that gemcitabine treatment of YAPS6A expressing Miapaca2 (FIG. 24A) or Panc02.13 (FIG. 24B) xenografts showed significantly reduced tumor growth in nude mice. Parental (left) or YAPS6A expressing Miapaca2 or Panc02.13 cells (right) were subcutaneously injected into athymic mice. When the outgrowths were approximately 200 mm3, mice were divided at random into two groups (vehicle control, gemcitabine). FIG. 24C depicts a bar graph showing relative levels of intra-tumor dFdU in Miapaca2 xenografts measured using LC/MS. FIG. 24D depicts graphs demonstrating that high levels of Hippo-YAP downstream gene target is associated with prolonged patient survival in pancreatic cancers in two independent studies. Kaplan-Meier curves of overall survival of pancreatic cancer patients with low or high levels of YAP-TEAD downstream targets.

FIGS. 25A-25C demonstrate that YAP activation sensitizes a panel of diverse human tumors to gemcitabine in PDX models. FIG. 25A demonstrates that high YAP expressing tumors shows significantly heightened sensitivity to gemcitabine (p=0.01, Mann-Whitney test). A plot showing tumor growth inhibition in response to gemcitabine in 20 PDX models. Tumor samples were stained with YAP levels and scored for high or low YAP index. Representative images of YAP staining among high and low YAP group are also shown. Scale bar, 200 μm. FIG. 25B depicts a graph of the poor correlation between gemcitabine response and tumor doubling time in PDX models (r=−0.07). FIG. 25C depicts plots showing tumor growth inhibition in response to other cytotoxic drugs is not affected by YAP levels (p>0.05).

FIG. 26 depicts schematics of the Hippo-YAP pathway, which mediates physiological resistance to gemcitabine. In low crowding conditions or in case of Hippo pathway genetic aberrations, Hippo pathway is inactive leading to lower levels of CDA and efflux pumps. This increases intracellular concentration of gemcitabine causing enhanced killing. In high crowding conditions, Hippo pathway is active leading to higher levels of CDA and efflux pumps. This reduces intracellular concentration of gemcitabine leading to drug resistance.

FIG. 27 depicts the inconsistency in gemcitabine response observed in literature for these cell lines. Literature curated gemcitabine IC50 in nanomolar.

FIG. 28 depicts pancreatic cancer cell lines with genetic and clinical characteristics used in the current study.

FIG. 29 depicts the presence of mutations/deletions in Hippo pathway genes in clinical studies of different cancer types.

FIG. 30 depicts characteristics of PDX models obtained from Champions TumorGraft® Database.

FIG. 31A depicts dose response curves of gemcitabine treated liver cancer and untransformed cell lines. The respective EC50 or for each cell line is also indicated. Growth factor stimulation of pancreatic cancer cells does not affect gemcitabine response. FIG. 31B depicts bar graphs showing changes in cell viability at 72 hr (top) and 96 hr (bottom) post stimulation with a combination of growth factor and gemcitabine. Cells were also treated with PBS control and gemcitabine alone. FIG. 31C demonstrates that growth factor stimulation activated their cognate downstream signaling proteins. Bar graphs showing activities of six downstream signaling proteins following stimulation with 15 growth factors. Series are, from left to right: PBS; Activin; BDNF; EGF; Ephb2; FGF; Gash; HGF; IGF; IL-6; PDFGb; PDFGb; PIGF; Tgfb; Wnt3a; and Wnt5a.

FIGS. 32A-32F demonstrate that changes in extrinsic factors do not affect gemcitabine response. FIG. 32A depicts a plot showing magnesium concentration increases cell growth in Bxpc3 cells in a dose-dependent manner. FIG. 32B demonstrates that high magnesium concentration (5 μM) has no effect on gemcitabine response in high crowding conditions. Bxpc3, Aspc1 and Panc10.05 cells grown in high crowding conditions were exposed to gemcitabine and cell viability was measured using live cell imaging. FIG. 32C demonstrates that conditioned media from Pancl or human dermal fibroblast (HDF) cells has no effect on gemcitabine response in high crowding conditions. FIG. 32D demonstrates that co-culturing of sparse GFP-labeled Pan02.13 cells achieved high overall cell density produced the same resistance to gemcitabine found in dense tumor cell culture. Cells grown in high crowding conditions do not acquire intrinsic resistance to apoptosis. FIG. 31E depicts a plot showing levels of 29 apoptosis-related signaling proteins in Panc02 cells grown in low crowding (LD) or high crowding conditions (HD). Levels of apoptotic proteins were measured using antibody arrays as described in materials and methods. FIG. 32F demonstrates that ultra-violet (UV)-induced apoptosis is not affected by cell crowding conditions. Panc02.13 cells grown in varying crowding conditions were exposed to medium strength UV for 10 sec. Cells were then lysed and whole cell lysates were subjected to western blotting. Western blots showing activities of cleaved caspase3, 7 and PARP.

FIGS. 33A-33F demonstrate cell crowding-dependent response to cytotoxic drugs in pancreatic cancer. FIG. 33A depicts plots showing the effect of six cytotoxic drugs on growth of seven pancreatic cancer cell lines under sparse and dense conditions. The efficacy of gemcitabine, doxorubicin was crowding-dependent while the effects of camptothecin paclitaxel, docetaxel and oxaliplatin were largely crowding-independent. Hippo-YAP pathway is activated in pancreatic cancer cells at high crowding conditions. FIG. 33B depicts a plot showing changes in phosphorylation of S6 ribosomal protein with cell crowding in six different pancreatic cancer cell lines. FIG. 33C depicts a heatmap showing changes in phosphorylation of growth factor signaling proteins such as Akt, Erk, Mek, Src, and S6 in Aspc1 cells. FIG. 33D depicts Western blots showing cell crowding-dependent changes in YAP phosphorylation (S127) in four pancreatic cancer cell lines. Knockdown of YAP decreases pancreatic cell proliferation. FIG. 33E depicts Western blots showing knockdown of YAP using two different shRNA in three pancreatic cell lines. Blots were also probed with β-actin for loading control. FIG. 33 F depicts plots showing growth of three pancreatic cancer cell lines expressing control or shRNA targeting YAP.

FIGS. 34A-34H demonstrate the cell crowding-dependent affect of verteporfin on pancreatic cancer cell growth. FIG. 34A depicts a graph demonstrating that verteporfin treatment potently slows down growth of Panc02.13 cells when grown in low crowding conditions. FIG. 34B depicts dose response curves of Panc02.13 cells treated with verteporfin, gemcitabine or combination of verteporfin and gemcitabine (50 nM) in a 3D-spheroid assay. EC50 of verteporfin in 3D-spheroid and low crowding condition is also indicated. FIG. 34C demonstrates that inactivation of Hippo pathway restores sensitivity to verteporfin in 3D-spheroid assay. Dose response curve of Panc02 cells expressing control-shRNA or shRNA targeting NF2. EC50 for each condition is also indicated. Hippo pathway inactivation mildly increases cell growth of pancreatic cancer cells. FIG. 34D depicts Western blots showing expression of V5-YAPS6A in Panc10.05 and Panc02.13 cells. FIG. 34E depicts Western blots showing expression of YAPS6A and NF2 knockdown increases phosphorylation of S6 ribosomal protein. Blots were also probed with β-actin for loading control. FIG. 34F depicts a plot showing mRNA expression of YAP-TEAD target genes in Panc02 cells expressing GFP or YAPS6A in high crowding conditions. FIG. 34G demonstrates that YAPS6A expression or NF2 depletion mildly increases cell growth in Panc02 cells. FIG. 34H depicts graphs of YAPS6A expression in Panc10.05 cells increases number of EdU-positive cell population in high crowding conditions.

FIGS. 35A-35H demonstrate that Hippo pathway inactivation sensitizes cells to gemcitabine and 5-FU. FIG. 35A demonstrates that Hippo inactivation (YAPS6A) expression sensitizes Panc02 cells to 5-FU in high crowding conditions. FIG. 35B demonstrates that YAPS6A expression increases apoptosis in gemcitabine treated Panc02 cells. Panc02 cells expressing YAPS6A or vector control were treated with varying doses of gemcitabine. Apoptosis was scored using nucview caspase 3/7 reagent. Plots show number of GFP positive (cleaved caspase3/7) cells upon gemcitabine treatment. FIG. 35C depicts a plot showing change in cell viability in gemcitabine treated Panc2 expressing vector or YAPS6A cells. FIG. 35D demonstrates that YAPS6A expression sensitizes cells to gemcitabine in a soft agar colony formation assay. FIG. 35E demonstrates that Hippo pathway inactivation increases action of several FDA-approved oncology drugs. Dose response curves of Panc02 cells expressing GFP or YAPS6A treated with 15 FDA-approved oncology drugs. FIG. 35F demonstrates that stability of gemcitabine in conditioned media over 5-day period. Plots showing gemcitabine and dFdU (FIG. 35G) from media-alone or from Panc02.13 cells collected over five days. Relative concentration of gemcitabine and dFdU was measured using LC/MS. FIG. 35H depicts representative Multiple-Reaction Monitoring (MRM) Chromatograms of gemcitabine and dFdU from Pan02 or media only at day 1.

FIGS. 36A-36M demonstrate that Hippo pathway inactivation decreases drug transport pumps. FIG. 36A depicts a bar graph showing relative mRNA expression of ABCB4, ABCC3 and MVP in Panc02.13 cells expressing control-shRNA or NF2-shRNA. FIG. 36B demonstrates that YAPS6A expression decreases expression of several transporters while the expression gemcitabine uptake pump (SLC29A1) remains unaffected. FIG. 36C depicts protein levels of LRP and ABCG2 in Panc02.13 cells expressing YAPS6A, or vector control or NF2-shRNA. FIG. 36D depicts Western blots showing cell crowding-dependent changes in protein levels of ABCG2 and LRP. FIG. 36E demonstrates that Hippo inactivation decreases levels of cytidine deaminase (CDA). YAPS6A expression in Pancl cells decreases mRNA expression of CDA. mRNA expression of dCK remains unaffected. FIG. 36F demonstrates that NF2 depletion in Patu8988S and YAPC cells decreases CDA levels. FIG. 36G depicts a Western blot showing expression of YAPS6A in Patu8902 cells decreases CDA protein levels. FIG. 36H demonstrates that verteporfin treatment increases mRNA expression of CDA in Panc02.13 cells. FIG. 36I demonstrates that gemcitabine resistant-MKN28 showed high levels of CDA. FIG. 36J depicts Western blots showing restoring LATS2 expresion in H2052 mesothelioma cells increases CDA protein levels. The levels of dCK remain unchanged. FIG. 36K demonstrates that LKB1 knockout cells showed decreased CDA levels. FIGS. 36L-36M depict plots showing normalized protein levels of phospho-YAP and CDA in A549 (STK11 mut) and Calu-1 (STK11-WT) cells under various crowding conditions.

FIGS. 37A-37G demonstrate that Hippo pathway inactivation correlates with better overall survival in pancreatic, lung and gastric cancers. FIG. 37A depicts a bar graph showing relative levels of cleaved caspase 7 and phosphor-H2aX in Miapaca2 xenografts. FIG. 37B depicts a Kaplan-Meier plot of lung cancer patients with low or high levels of CTGF. FIG. 37C depicts a Kaplan-Meier plots of gastric cancer patients treated with 5-FU-based chemotherapy with Hippo activation (levels of NF2, left) or hippo inactivation (levels of CTGF, right). FIG. 37D depicts Kaplan-Meier plots sowing overall survival of pancreatic cancer patients with low or high levels of Hippo-YAP independent transporter gene signature. FIG. 37E demonstrates that drug modulating pumps and CDA levels are upregulated in pancreatic cancers. Plots showing increased relative expresion levels of ABCC3, MVP and (FIG. 37F) CDA in pancreatic tumor samples compared with normal tissue. FIG. 37G demonstrates that levels of YAP-TEAD target genes are not altered in pancreatic tumor samples.

### DETAILED DESCRIPTION

As described herein, the inventors have demonstrated that the sensitivity of cancer cells to certain chemotherapeutics (e.g. gemcitabine, camptothecin, and 5-FU) is dependent on cell-to-cell contact, e.g. cell density. In particular, the cells are more resistant at higher densities. However, inhibition of the Hippo signaling pathway suppresses this resistance, restoring sensitivity in both 2D and 3D cultures. Accordingly, provided herein are methods of diagnosing, prognosing, and treating cancer that relate to the alteration of sensitivity to chemotherapeutics by the Hippo pathway.

As described herein, the inventors have demonstrated that cells with decreased activity of the Hippo-YAP signaling pathway are sensitive to certain chemotherapeutics, e.g. gemcitabine, camptothecin, and 5-FU. Additionally, 119 FDA-approved oncology drugs were screened for their ability to inhibit spheroid cell growth in both Hippo active and parental pancreatic cancer cell lines in accordance with the assays described in the Examples herein. A number of compounds were identified that have particularly significant inhibitory activity when the Hippo-YAP pathway activity is decreased (i.e., when YAP is activated and localized to the nucleus). Those compounds include cladribine (a purine analog approved for hairy cell leukemia, AML, and ALL); mitoxantrone (a type II topoisomerase approved for AML, non-Hodgkin's lymphoma and metastatic breast cancers); methotrexate (an antifolate drug approved for leukemia, lymphoma, lung, and osteosarcoma); irrenotecan; etoposide; and teniposide.

Accordingly, in one aspect of any of the embodiments described herein, is a method of treating cancer by administering a chemotherapeutic selected from the group consisting of: an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; to a subject having cancer cells with decreased Hippo-YAP signaling pathway activity and/or cancer cells not having upregulating Hippo-YAP signaling pathway activity. In some embodiments, the chemotherapeutic can be selected from the group consisting of: an antimetabolite; a nucleoside analog; an antifolate; a topoisomerase I inhibitor; a topoisomerase II inhibitor; an anthracycline; a tubulin modulator; and a DNA cross-linking agent. In some embodiments, the chemotherapeutic can be selected from the group consisting of: gemcitabine; 5-FU; cladribine; cytarabine; tioguanine; mercaptopurine; clofarabine; methotrexate; camptothecin; topotecan; irrenotecan; epirubicin; daunorubicin; doxorubicin; valrubicin; teniposide; etopiside; mitoxantrone; ixabepilone; imatinib; and mitomycin.

In one aspect of any of the embodiments described herein is a method of treating cancer, the method comprising administering a chemotherapeutic selected from the group consisting of: an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; to a subject having cancer cells determined to have: a) a deletion, a truncation or inactivating mutation in FAT4; LATS1; LATS2; STK11; or NF2; b) decreased expression of FAT4; LATS1; LATS2; STK11; or NF2 relative to a reference; c) increased expression of YAP; CTGF; AREG; AMOTL2; AXL; or BIRC5 relative to a reference; d) decreased phosphorylation of YAP relative to a reference; or e) increased nuclear localization of YAP relative to a reference. In some embodiments, the chemotherapeutic can be selected from the group consisting of: an antimetabolite; a nucleoside analog; an antifolate; a topoisomerase I inhibitor; a topoisomerase II inhibitor; an anthracycline; a tubulin modulator; and a DNA cross-linking agent. In some embodiments, the chemotherapeutic can be selected from the group consisting of: gemcitabine; 5-FU; cladribine; cytarabine; tioguanine; mercaptopurine; clofarabine; methotrexate; camptothecin; topotecan; irrenotecan; epirubicin; daunorubicin; doxorubicin; valrubicin; teniposide; etopiside; mitoxantrone; ixabepilone; imatinib; and mitomycin.

Additionally, susceptibility to a chemotherapeutic selected from the group consisting of: an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; can also be induced by inhibiting Hippo-YAP signaling. Accordingly, provided herein is a method of treating cancer comprising administerting, to a subject in need of treatment thereof, i) a chemotherapeutic selected from the group consisting of: an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; and ii) an inhibitor of Hippo-YAP signaling, e.g., an inhibitor of FAT4; STK11; LATS1; LATS2; or NF2; or an agonist of YAP. In some embodiments, the chemotherapeutic can be selected from the group consisting of: an antimetabolite; a nucleoside analog; an antifolate; a topoisomerase I inhibitor; a topoisomerase II inhibitor; an anthracycline; a tubulin modulator; and a DNA cross-linking agent. In some embodiments, the chemotherapeutic can be selected from the group consisting of: gemcitabine; 5-FU; cladribine; cytarabine; tioguanine; mercaptopurine; clofarabine; methotrexate; camptothecin; topotecan; irrenotecan; epirubicin; daunorubicin; doxorubicin; valrubicin; teniposide; etopiside; mitoxantrone; ixabepilone; imatinib; and mitomycin.

Chemotherapeutics selected from the group consisting of: an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; are known in the art and are readily identified by one of skill in the art. An antimetabolite chemotherapeutic is an agent that inhibits the use of a metabolite, e.g., the use of folic acid or nucleosides or nucleotides. Antimetabolites can include, e.g. nucleoside analogs and antifolates. Nucleoside analogs are compounds that mimic the structure of a natural nucleoside such that attempts to incorporate them in DNA or RNA synthesis inhibits further synthesis. By way of non-limiting example, the nucleoside analog can be gemcitabine; 5-FU; cladribine; cytarabine; tioguanine; mercaptopurine; clofarabine; or a variant or derivative thereof. Antifolates mimic the structure of folic acid such that they inhibit metabolism of folic acid. By way of non-limiting example, the antifolate can be methotrexate or a variant or derivative thereof.

Topoisomerase inhibitors are compounds that inhibit the activity of one or more topoisomerases, e.g, topoisomerase I or II. By way of non-limiting example, the topoisomerase I inhibitor can be camptothecin, topotecan, irrenotecan, or a variant or derivative thereof. By way of non-limiting example, the topoisomerase II inhibitor can be epirubicin; daunorubicin; doxorubicin; valrubicin; teniposide; etopiside; mitoxantrone, or a variant or derivative thereof. In some embodiments of any of the aspects described herein, the topoisomerase II inhibitor can be an inihibitor that is not an anthracycline. By way of non-limiting example, the topoisomerase II inhibitor that is not an anthracycline can be teniposide; etopiside; mitoxantrone; or a variant or derivative thereof. Anthracylcines are a structural class of compounds derived from Streptomyces. Anthracyclines can include, e.g., epirubicin; daunorubicin; doxorubicin; valrubicin, or a variant or derivative thereof.

A tubulin modulator is an agent that modulates the synthesis, assembly, or disassembly of tubulin and/or microtubules. In some embodiments of any of the aspects described herein, the tubulin modulator can stabilize microtubules. By way of non-limiting example, the tubulin modulator can be ixabepilone. A DNA cross-linking agent is an agent that can induce cross-links in DNA, e.g., via alkylation. Such cross-links inhibit DNA and RNA synthesis. By way of non-limiting example, a DNA cross-linking agents can include mitomycin. Src family kinase inhibitors are tyrosine kinase inhibitor agents that inhibit the activity (e.g., reduce the phosphorylation of a target molecule) of one or more Src family kinases (e.g., Src, Yes, Fyn, Fgr, Lck, Hck, Blk, Lyn, and Frk). By way of non-limiting example, Src family kinase inhibitors can include imatinib. BCR-Abl kinase inhibitors are tyrosine kinase inhibitor agents that inhibit the activity (e.g., reduce the phosphorylation of a target molecule) of BCR-Abl. By way of non-limiting example, BCR-Abl kinase inhibitors can include imatinib.

As used herein, “Hippo-YAP signaling pathway” refers to a signaling pathway involving a kinase cascade that regulates, e.g. drug transporter expression. The pathway comprises FAT4, which is an upstream regulator of the pathway and may act as a receptor; NF2, which is an upstream regulator of the pathway; the serine/threonine kinase STK11; and LATS1/2, nuclear DBF-2 related kinases which, when active, suppress the activity of YAP by phosphorylation. Thus, when the Hippo-YAP pathway is active, YAP is phosphorylated, e.g., at Ser127, preventing its translocation to the nucleus and maintaining it in an inactive form. When the Hippo-YAP pathway is downregulated, YAP is activated by being dephosphorylated and localized to the nucleus. When YAP is active, it leads to the downregulation of several multidrug transporters (e.g., ABCG2, ABCC3, and LRP). As described herein, the Hippo-YAP pathway is downregulatedwhen cells are at low density and is upregulated when cells are in high density conditions.

As used herein, “FAT4” or “FAT atypical cadherin 4” refers to a member of the Hippo-YAP pathway that may function as a receptor. Nucleic acid and polypeptide sequences for FAT4 are known for a number of species, e.g., human FAT4 (NCBI Gene ID: 79663; NM_001291303 (mRNA)(SEQ ID NO: 1); and NP_001278232 polypeptide (SEQ ID NO: 2)).

As used herein, “STK11” or “serine threonine kinase 11” refers to a kinase of the Hippo-YAP signaling cascade. Nucleic acid and polypeptide sequences for STK11 are known for a number of species, e.g., human STK11 (NCBI Gene ID: 6794; NM_000455 (mRNA)(SEQ ID NO: 3); and NP 000446 polypeptide (SEQ ID NO: 4)).

As used herein, “LATS1” or “large tumor suppressor kinase 1” refers to a kinase that promotes the phosphorylation of YAP. Nucleic acid and polypeptide sequences for LATS1 are known for a number of species, e.g., human LATS1 (NCBI Gene ID: 9113; NM_004690 (mRNA)(SEQ ID NO: 5); and NP 00468 polypeptide (SEQ ID NO: 6)).

As used herein, “LATS2” or “large tumor suppressor kinase 2” refers to a kinase that promotes phosphorylation of YAP. Nucleic acid and polypeptide sequences for LATS2 are known for a number of species, e.g., human LATS2 (NCBI Gene ID: 26524; NM_014572 (mRNA)(SEQ ID NO: 7); and NP 055387 polypeptide (SEQ ID NO: 8)).

As used herein, “NF2” or “neurofibromin 2” refers to an upstream regulator in the Hippo pathway that is required for LATS1/2 phosphorylation of YAP. Nucleic acid and polypeptide sequences for NF2 are known for a number of species, e.g., human NF2 (NCBI Gene ID: 4771; NM_000268 (mRNA)(SEQ ID NO: 9); and NP_000259 polypeptide (SEQ ID NO: 10)).

As used herein, “YAP” or “YES-associated protein 1” refers to a member of the Hippo pathway, that when active, translocates to the nucleus to regulate gene transcription. Nucleic acid and polypeptide sequences for YAP are known for a number of species, e.g., human YAP (NCBI Gene ID: 10413; NM_001282101 (mRNA)(SEQ ID NO: 11); and NP_001269030 polypeptide (SEQ ID NO: 12)). When YAP is dephosphorylated, it is translocated to the nucleus and interacts with transcription factors to regulate expression of a number of genes, e.g., as described elsewhere herein. Accordingly, decreased activity of the Hippo-YAP pathway can be indicated by decreased levels of phosphorylation of YAP and/or increased nuclear levels of YAP.

Active YAP can modulate the expression of CTGF; AREG; AMOTL2; AXL; and BIRC5, such that increased expression and/or activity of YAP results in increased expression and/or activity of CTGF (e.g. NCBI Gene ID: 1490); AREG (e.g. NCBI Gene ID: 374); AMOTL2 (NCBI Gene ID: 51421); AXL (NCBI Gene ID: 558); and/or BIRC5 (NCBI Gene ID: 332). Nucleic acid and polypeptide sequences for the foregoing genes are known for a number of species, e.g., the human sequences associated with the provided accession numbers.

In some embodiments, measurement of the level of a target and/or detection of the level or presence of a target, e.g. of an expression product (nucleic acid or polypeptide of one of the genes described herein) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments, a DNA sample can undergo enzymatic replication, e.g., by polymerase chain reaction (PCR).

Transformation, measurement, and/or detection of a target molecule, e.g. a YAP mRNA or polypeptide can comprise contacting a sample obtained from a subject with a reagent (e.g. a detection reagent) which is specific for the target, e.g., a target-specific reagent. In some embodiments, the target-specific reagent is detectably labeled. In some embodiments, the target-specific reagent is capable of generating a detectable signal. In some embodiments, the target-specific reagent generates a detectable signal when the target molecule is present.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for the various targets described herein are commercially available and can be used for the purposes of the invention to measure protein expression levels, e.g. anti-YAP (Cat. No. ab52771; Abcam, Cambridge Mass.). Alternatively, since the amino acid sequences for the targets described herein are known and publically available at the NCBI website, one of skill in the art can raise their own antibodies against these polypeptides of interest for the purpose of the invention.

The amino acid sequences of the polypeptides described herein have been assigned NCBI accession numbers for different species such as human, mouse and rat. In particular, the NCBI accession numbers for the amino acid sequence of human YAP is included herein, e.g. SEQ ID NO: 12.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments, the immunoassay can be a quantitative or a semi-quantitative immunoassay.

An immunoassay is a biochemical test that measures the concentration of a substance in a biological sample, typically a fluid sample such as urine, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., any of the targets as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3, 3′, 5, 5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce significant color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick tests, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In some embodiments, the level of a target can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the gene expression products as described herein can be instead determined by determining the level of messenger RNA (mRNA) expression of the genes described herein. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNAse protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

The nucleic acid sequences of the genes described herein have been assigned NCBI accession numbers for different species such as human, mouse and rat. For example, the human YAP mRNA (e.g. SEQ ID NO: 11) is known. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments, detecting decreased activity and/or expression of a target can comprise detecting the present of a deletion, a truncation or inactivating mutation, i.e. a mutation that decreases the activity and/or level of the gene products expressed from the gene. A number of such mutations are known in the art and are provided in Table 2 herein.

In some embodiments, the assays and methods can relate to detecting the presence of a mutation, e.g. a deletion, a truncation or inactivating mutation in a sample obtained from a subject. In some embodiments, the presence of the mutation can be determined using an assay selected from the group consisting of: hybridization; sequencing; exome capture; PCR; high-throughput sequencing; allele-specific probe hybridization; allele-specific primer extension, allele-specific amplification; 5′ nuclease digestion; molecular beacon assay; oligonucleotide ligation assay; size analysis; single-stranded conformation polymorphism; real-time quantitative PCR, and any combinations thereof.

In some embodiments, the presence and/or absence of a mutation can be detected by determining the sequence of a genomic locus and/or an mRNA transcript. Such molecules can be isolated, derived, or amplified from a biological sample, such as a tumor sample. Nucleic acid (e.g. DNA) and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; and proteinase K extraction can be used to obtain nucleic acid from blood (Roiff, A et al. PCR: Clinical Diagnostics and Research, Springer (1994)).

In some embodiments, the nucleic acid sequence of a target gene in a sample obtained from a subject can be determined and compared to a reference sequence to determine if a mutation is present in the subject. In some embodiments, the sequence of the target gene can be determined by sequencing the target gene (e.g. the genomic sequence and/or the mRNA transcript thereof). Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (3 ed.), Cold Spring Harbor Laboratory Press, Cold Spring Harbor, N.Y., USA (2001); which are incorporated by reference herein in their entireties.

In some embodiments, sequencing can comprise exome sequencing (i.e. targeted exome capture). Exome sequencing comprises enriching for an exome(s) of interest and then sequencing the nucleic acids comprised by the enriched sample. Sequencing can be according to any method known in the art, e.g. those described above herein. Methods of enrichment can include, e.g. PCR, molecular inversion probes, hybrid capture, and in solution capture. Exome capture methodologies are well known in the art, see, e.g. Sulonen et la. Genome Biology 2011 12:R94; and Teer and Mullikin. Hum Mol Genet 2010 19:R2; which are incorporated by reference herein in their entireties. Kits for performing exome capture are available commercially, e.g. the TRUSEQ™ Exome Enrichment Kit (Cat. No. FC-121-1008; Illumnia, San Diego, Calif.). Exome capture methods can also readily be adapted by one of skill in the art to enrich specific exomes of interest.

In some embodiments, the presence of a mutation can be determined using a probe that is specific for the mutation. In some embodiments, the probe can be detectably labeled. In some embodiments, a detectable signal can be generated by the probe when a mutation is present.

In some embodiments, the probe specific for the mutation can be a probe in a hybridization assay, i.e. the probe can specifically hybridize to a nucleic acid comprising a mutation (as opposed to a wild-type nucleic acid sequence) and the hybridization can be detected, e.g. by having the probe and or the target nucleic acid be detectably labeled. Hybridization assays are well known in the art and include, e.g. northern blots and Southern blots.

In some embodiments, the probe specific for the mutation can be a probe in a PCR assay, i.e. a primer. In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a thermostable DNA polymerase, and optionally, (iii) screening the PCR products for a band or product of the correct size. The primers used are oligonucleotides of sufficient length and appropriate sequence to provide initiation of polymerization, i.e. each primer is specifically designed to be complementary to a strand of the genomic locus to be amplified. In an alternative embodiment, the presence of a mutation in an mRNA tramscript can be determined by reverse-transcription (RT) PCR and by quantitative RT-PCR (QRT-PCR) or real-time PCR methods. Methods of RT-PCR and QRT-PCR are well known in the art. In some embodiments, the PCR product can be labeled, e.g. the primers can comprise a detectable label, or a label can be incorporated and/or bound to the PCR product, e.g. EtBr detection methods. Other non-limiting detection methods can include the detection of a product by mass spectroscopy or MALDI-TOF.

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylene diaminetetraacetic acid (EDTA).

A level which is less than a reference level can be a level which is less by at least about 10%, at least about 20%, at least about 50%, at least about 60%, at least about 80%, at least about 90%, or less than the reference level. In some embodiments, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments, a level which is more than a reference level can be a level which is statistically significantly greater than the reference level.

In some embodiments, the reference can be a level of the target molecule in a population of subjects who do not have or are not diagnosed as having, and/or do not exhibit signs or symptoms of a cancer. In some embodiments, the reference can also be a level of expression of the target molecule in a control sample, a pooled sample of control individuals or a numeric value or range of values based on the same. In some embodiments, the reference can be the level of a target molecule in a sample obtained from the same subject at an earlier point in time, e.g., the methods described herein can be used to determine if a subject's sensitivity to a given therapy is changing over time.

In some embodiments, the level of expression products of no more than 200 other genes is determined. In some embodiments, the level of expression products of no more than 100 other genes is determined. In some embodiments, the level of expression products of no more than 20 other genes is determined. In some embodiments, the level of expression products of no more than 10 other genes is determined.

In some embodiments of the foregoing aspects, the expression level of a given gene can be normalized relative to the expression level of one or more reference genes or reference proteins.

The term “sample” or “test sample” as used herein denotes a sample taken or isolated from a biological organism, e.g., a blood or plasma sample from a subject. Exemplary biological samples include, but are not limited to, a biopsy, a tumor sample, biofluid sample; serum; plasma; urine; saliva; and/or tissue sample etc. The term also includes a mixture of the above-mentioned samples. The term “test sample” also includes untreated or pretreated (or pre-processed) biological samples. In some embodiments, a test sample can comprise cells from a subject. In some embodiments, the test sample can be a biopsy, tumor sample, blood; plasma; urine, or serum.

The test sample can be obtained by removing a sample from a subject, but can also be accomplished by using a previously isolated sample (e.g. isolated at a prior timepoint and isolated by the same or another person).

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein can further comprise a step of obtaining a test sample from a subject. In some embodiments, the subject can be a human subject. In some embodiments, the subject can be a subject in need of treatment for (e.g. having or diagnosed as having) a cancer or a subject at risk of or at increased risk of developing a cancer as described elsewhere herein.

In some embodiments of any of the aspects described herein, a method of treatment can further comprise a step of detecting and/or measuring the level of a Hippo-YAP pathway gene product (e.g. a nucleic acid or polypeptide) as described herein (e.g. FAT4; LATS1; LATS2; STK11; NF2; YAP; CTGF; AREG; AMOTL2; AXL; and/or BIRC5); the level of phosphylation and/or level of nuclear localization of YAP; and/or the presence of a deletion, a truncation or an inactivating mutation of FAT4, LATS1, LATS2, STK11, and/or NF2.

As used herein, the term “inhibitor” refers to an agent which can decrease the expression and/or activity of the targeted expression product, e.g. by at least 10% or more, e.g. by 10% or more, 50% or more, 70% or more, 80% or more, 90% or more, 95% or more, or 98% or more. The efficacy of an inhibitor of a particularly target e.g. its ability to decrease the level and/or activity of the target can be determined, e.g. by measuring the level of an expression product and/or the activity of the target. Methods for measuring the level of a given mRNA and/or polypeptide are known to one of skill in the art, e.g. RT-PCR with primers can be used to determine the level of RNA and Western blotting with an antibody (e.g. an anti-FAT4 antibody, e.g. Cat No. ab130076; Abcam; Cambridge, Mass.) can be used to determine the level of a polypeptide. The activity of a target can be determined using methods known in the art, e.g. measuring the expression level of a gene regulated by the Hippo-YAP pathway or the level of phosphorylation of a downstream member of the pathway as described herein. In some embodiments, the inhibitor can be an inhibitory nucleic acid; an aptamer; an antibody reagent; an antibody; or a small molecule.

Small molecule inhibitors of the targets described herein, e.g., FAT4, LATS1, LATS2, STK11, and NF2, are known in the art. By way of non-limiting example, AZ-23 is an inhibitor of STK11 and LATS2 inhibitors can include GSK690693, AT7867, and PF-477736.

As used herein, an agonist refers to any agent that increases the level and/or activity of the target, e.g, of YAP. As used herein, the term “agonist” refers to an agent which increases the expression and/or activity of the target by at least 10% or more, e.g. by 10% or more, 50% or more, 100% or more, 200% or more, 500% or more, or 1000% or more. The efficacy of an agonist of, for example, YAP, e.g. its ability to increase the level and/or activity of YAP be determined, e.g. by measuring the level of an expression product of YAP and/or the activity of YAP. Methods for measuring the level of a given mRNA and/or polypeptide are known to one of skill in the art, e.g. RTPCR with primers can be used to determine the level of RNA, and Western blotting with an antibody (e.g. an anti-YAP antibody, e.g. Cat No. ab52771 Abcam; Cambridge, Mass.) can be used to determine the level of a polypeptide. The activity of, e.g. YAP can be determined using methods described elsewhere herein, e.g. by measuring the level of phosphorylation or the localization of YAP to the nucleus, and/or by measuring the level of gene expression of known targets of YAP, e.g., BIRC5 or other targets described herein.

Non-limiting examples of agonists of YAP can include YAP polypeptides or fragments thereof and nucleic acids encoding a YAP polypeptide, e.g. a polypeptide comprising the sequence SEQ ID NO: 12 or a nucleic acid comprising the sequence of SEQ ID NO: 11 or variants thereof. In some embodiments, the agonist of YAP can be an YAP polypeptide. In some embodiments, the agonist of YAP can be an engineered and/or recombinant polypeptide. In some embodiments, the agonist of YAP can be a nucleic acid encoding YAP, e.g. a functional fragment thereof. As described above herein, a decrease (or lack) of phosphorylation of YAP induces its translocation to the nucleus where it is active. Accordingly, in some embodiments of any of the aspects described herein, the agonist of YAP can be a non-phospho, active form of YAP (e.g. a form of YAP comprising one or more mutations selected from S61A, S109A, S127A, S128A, S131A, S163A, S164A, S381A (e.g. relative to SEQ ID NO: 12) or a nucleic acid encoding such a non-phospho, active form of YAP. In some embodiments of any of the aspects described herein, the nucleic acid can be comprised by a vector.

In the screen of 119 FDA-approved oncology drugs described above herein, several drugs were identified that were effective in preventing cancer cell growth independent of the state of Hippo-YAP signaling pathway activity, e.g., these compounds are effective even when the Hippo-YAP pathway is active. Those compounds include, e.g., carfilzomib; bortezomib; dactinomycin; plicamycin; ponatinib; trametinib; enzalutamide; and omacetaxine mepesuccinate.

Accordingly, in one aspect of any of the embodiments described herein, is a method of treating cancer, the method comprising administering a chemotherapeutic selected from the group consisting of: an antimetabolite; an anthracylcine; an anthracycline topoisomerase II inhibitor; a proteasome inhibitor; an mTOR inhibitor; an RNA synthesis inhibitor; a peptide synthesis inhibitor; an alkylating agent; an antiandrogen; a Src family kinase inhibitor; a BCR-Abl kinase inhibitor; a MEK inhibitor; and a kinase inhibitor; to a subject having cancer cells determined not to have: a) a deletion, a truncation, or inactivating mutation in FAT4; LATS1; LATS2; STK11; or NF2; b) decreased expression of FAT4; LATS1; LATS2; STK11; or NF2 relative to a reference; c) increased expression of YAP; CTGF; AREG; AMOTL2; AXL; or BIRC5 relative to a reference; d) decreased phosphorylation of YAP relative to a reference; or e) increased nuclear localization of YAP relative to a reference. In some embodiments, the subject can have cancer cells determined not to have: a) a deletion, a truncation, or inactivating mutation in FAT4; LATS1; LATS2; STK11; or NF2; b) decreased expression of FAT4; LATS1; LATS2; STK11; or NF2 relative to a reference; c) increased expression of YAP; CTGF; AREG; AMOTL2; AXL; or BIRC5 relative to a reference; d) decreased phosphorylation of YAP relative to a reference; and e) increased nuclear localization of YAP relative to a reference.

In some embodiments, the chemotherapeutic can be selected from the group consisting of an antimetabolite; a proteasome inhibitor; an RNA synthesis inhibitor; a peptide synthesis inhibitor; an antiandrogen; a Src family kinase inhibitor; a BCR-Abl kinase inhibitor; a MEK inhibitor; and a kinase inhibitor. In some embodiments, the chemotherapeutic can be selected from the group consisting of an antimetabolite; a proteasome inhibitor; an RNA synthesis inhibitor; a peptide synthesis inhibitor; an antiandrogen; and a MEK inhibitor. In some embodiments, the chemotherapeutic can be selected from the group consisting of an antimetabolite; a proteasome inhibitor; a peptide synthesis inhibitor; an antiandrogen; and a MEK inhibitor. In some embodiments, the chemotherapeutic can be selected from the group consisting of: daunorubicin; doxorubicin; epirubicin; valrubicin; carfilzomib; bortezomib; everolimus; triethylenemelamine; dactinomycin; plicamycin; ponatinib; trametinib; enzalutamide; and omacetaxine mepesuccinate. In some embodiments, the chemotherapeutic can be selected from the group consisting of: daunorubicin; doxorubicin; epirubicin; valrubicin; carfilzomib; bortezomib; dactinomycin; plicamycin; ponatinib; trametinib; enzalutamide; and omacetaxine mepesuccinate. In some embodiments, the chemotherapeutic can be selected from the group consisting of: carfilzomib; bortezomib; dactinomycin; plicamycin; ponatinib; trametinib; enzalutamide; and omacetaxine mepesuccinate.

Chemotherapeutics which are an antimetabolite; an anthracylcine; an anthracycline topoisomerase II inhibitor; a proteasome inhibitor; an mTOR inhibitor; an RNA synthesis inhibitor; a peptide synthesis inhibitor; an alkylating agent; an antiandrogen; a Src family kinase inhibitor; a BCR-Abl kinase inhibitor; a MEK inhibitor; or a kinase inhibitor are known in the art and readily identified by one of skill in the art. By way of non-limiting example, a anthracycline toposisomerase II inhibitor can be daunorubicin; doxorubicin; epirubicin; valrubicin; or a variant or derivative thereof. A proteasome inhibitor is an agent that inhibits the activity of the proteasome (e.g., protein degradation). By way of non-limiting example, proteasome inhibitors can include carfilzomib, bortezomib, or a variant or derivative thereof. mTOR inhibitors are agents that inhibit the activity of mTOR (e.g. the mTORC1 and/or mTORC2 complexes). By way of non-limiting example, mTOR inhibitors can include everolimus or a variant or derivative thereof. RNA synthesis inhibitors are agents that inhibit the synthesis of mRNA molecules, e.g., they inhibit transcription. In some embodiments, RNA synthesis inhibitors inhibit synthesis by binding to a component of the RNA polymerase complex. By way of non-limiting example, RNA synthesis inhibitors can include triethylenemelamine, dactinomycin, plicamycin, or a variant or derivative thereof. A peptide synthesis inhibitor is an agent that inhibits the synthesis of polypeptides, e.g., that inhibits translation. By way of non-limiting example, peptide synthesis inhibitors can include omacetaxine mepesuccinate. Antiandrogens are compounds that inhibit androgen-dependent signaling, e.g., by competing for binding to androgen receptors. By way of non-limiting example, antiandrogens can include enzalutamide. By way of non-limiting example, alkylating agents can include triethylenemelamine. By way of non-limiting example, a Src family kinase inhibitor or BCR-Abl kinase inhibitor can include ponatinib. MEK inhibitors are agents that inhibit the activity of mitogen-activated protein kinase kinase enzyme MEK1 and/or MEK2. By way of non-limiting example, MEK inhibitors can include trametinib.

In some embodiments of any of the aspects described herein, the cancer can be pancreatic cancer; pancreatic ductal adenocarcinoma; metastatic breast cancer; breast cancer; bladder cancer; small cell lung cancer; lung cancer; ovarian cancer; stomach cancer; uterine cancer; mesothelioma; adenoid cystic carcinoma; lymphoid neoplasm; kidney cancer; colorectal cancer; adenoid cystic carcinoma; prostate cancer; cervical cancer; head and neck cancer; or glioblastoma. In some embodiments of any of the aspects described herein, the cancer can be pancreatic cancer.

In some embodiments, the methods described herein relate to treating a subject having or diagnosed as having cancer. Subjects having cancer can be identified by a physician using current methods of diagnosing cancer. Symptoms and/or complications of cancer, e.g. pancreatic cancer, which characterize these conditions and aid in diagnosis are well known in the art and include but are not limited to, pain in the upper abdomen, jaundice, weight loss, digestive problems, or diabetes. Tests that may aid in a diagnosis of, e.g. pancreatic cancer include, but are not limited to, CT scane, endoscopic ultrasound, biopsy, liver function tests, MRI, and/or PET. A family history of cancer or exposure to risk factors for cancer (e.g. in the case of pancreatic cancer, having diabetes) can also aid in determining if a subject is likely to have cancer or in making a diagnosis of cancer.

The compositions and methods described herein can be administered to a subject having or diagnosed as having cancer. In some embodiments, the methods described herein comprise administering an effective amount of compositions described herein, e.g. an agonist of YAP to a subject in order to alleviate a symptom of a cancer. As used herein, “alleviating a symptom of a cancer” is ameliorating any condition or symptom associated with the cancer. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a composition (e.g. an agonist of YAP) needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular anti-tumor effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for Hippo-YAP signaling activity and/or tumor growth, among others. The dosage can be determined by a physician and adjusted, as necessary, to suit observed effects of the treatment.

In some embodiments, the technology described herein relates to a pharmaceutical composition comprising a chemotherapeutic and/or agonist of YAP as described herein, and optionally a pharmaceutically acceptable carrier. In some embodiments, the active ingredients of the pharmaceutical composition comprise an agent (e.g., a chemotherapeutic and/or agonist of YAP) as described herein. In some embodiments, the active ingredients of the pharmaceutical composition consist essentially of, e.g., a chemotherapeutic and/or agonist of YAP as described herein. In some embodiments, the active ingredients of the pharmaceutical composition consist of, e.g., a chemotherapeutic and/or agonist of YAP, as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, as described herein.

In some embodiments, the pharmaceutical composition comprising, e.g., a chemotherapeutic and/or agonist of YAP, as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS-type dosage forms and dose-dumping.

Suitable vehicles that can be used to provide parenteral dosage forms are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an active ingredient as disclosed herein can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering an additional agent and/or treatment to the subject, e.g. as part of a combinatorial therapy. Non-limiting examples of a second agent and/or treatment can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy. Further, the methods of treatment can further include the use of surgical treatments.

In certain embodiments, an effective dose of a composition, e.g. a composition comprising a chemotherapeutic and/or agonist of YAP as described herein, can be administered to a patient once. In certain embodiments, an effective dose of a composition can be administered to a patient repeatedly. For systemic administration, subjects can be administered a therapeutic amount of a composition, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. reduce tumor growth and/or size by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of, e.g., a chemotherapeutic and/or agonist of YAP, according to the methods described herein depend upon, for example, the form of the active ingredient, its potency, and the extent to which symptoms, markers, or indicators of a condition described herein are desired to be reduced, for example the percentage reduction desired for tumor growth or the extent to which, for example, YAP activity are desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition, e.g, a chemotherapeutic and/or agonist of YAP, in, e.g. the treatment of a condition described herein, or to induce a response as described herein (e.g. YAP activation) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor growth or YAP activity. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or tumor growth); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. YAP activity). It is well within the ability of one skilled in the art to monitor efficacy of administration and/or treatment by measuring any one of such parameters, or any combination of parameters. Efficacy can be assessed in animal models of a condition described herein, for example treatment of mouse models of pancreatic cancer. When using an experimental animal model, efficacy of treatment is evidenced when a statistically significant change in a marker is observed, e.g. tumor growth, liver function, and/or Hippo-YAP signaling activity.

In vitro and animal model assays are provided herein which allow the assessment of a given dose of a a chemotherapeutic and/or agonist of YAP. By way of non-limiting example, the effects of a dose of a given agent can be assessed by measuring the nuclear localization of YAP. A non-limiting example of a protocol for such an assay is as follows: Panc02.13 cells can be cultured on Lab-Tek II™ chamber glass slides (Nalge Nunc, Naperville, Ill.) or on 24-well glass bottom dishes (MatTek Corporation). Cells are fixed in 4% paraformaldehyde for 15 min at room temperature, washed in PBS, permeabilized with 0.1% Triton X-100, and blocked for 60 min with PBS containing 3% BSA (w/v). Cells are immunostained with the appropriate antibody (e.g. anti-YAP antibody), following by immunostaining with Alexa Fluor 488-labeled goat-anti-rabbit antibody (Molecular Probes, Eugene, Oreg.). Nuclei are counterstained with Hoescht 33342 (Sigma-Aldrich, St. Louis, Mo.). Fluorescent micrographs can be obtained using a Nikon A1R™ point scanning confocal microscope. Individual channels were overlaid using ImageJ™ software (National Institutes of Health, Bethesda, Md.)

In one aspect of any of the embodiments described herein, provided herein is an assay comprising detecting, in a test sample obtained from a subject in need of treatment for cancer; i) a deletion, a truncation or inactivating mutation in FAT4; LATS1; LATS2; STK11; or NF2; ii) decreased expression of FAT4; LATS1; LATS2; STK11; or NF2 relative to a reference; iii) increased expression of YAP; CTGF; AREG; AMOTL2; AXL; or BIRC5 relative to a reference; iv) decreased phosphorylation of YAP relative to a reference; and/or v) increased nuclear localization of YAP relative to a reference. wherein the presence of any of i)-v) indicates the subject is more likely to respond to treatment with a nucleoside analog; an antifolate; a topoisomerase I inhibitor; and a topoisomerase II inhibitor that is not an anthracycline.

In some embodiments of any of the aspects described herein, the absence of any of i)-v) indicates the subject should receive treatment with a treatment selected from the group consisting of: daunorubicin; doxorubicin; Epirubicin; Valrubicin; Carfilzomib; Dactinomycin; Everolimus; Plicamycin; Triethylenemelamine; and/or Ponatinib. In some embodiments of any of the aspects described herein, the absence of i)-v) indicates the subject should receive treatment with a treatment selected from the group consisting of: daunorubicin; doxorubicin; Epirubicin; Valrubicin; Carfilzomib; Dactinomycin; Everolimus; Plicamycin; Triethylenemelamine; and/or Ponatinib.

In some embodiments of any of the aspects described herein, the methods, assays, and systems described herein can comprise creating a report based on results of the determining and/or measuring step. In some embodiments, the report denotes raw values for the levels of a marker gene or gene expression product in the sample (plus, optionally, the level in a reference sample) or it indicates a percentage or fold increase in the level as compared to a reference level, and/or provides a signal indicating what treatments should or should not be administered to the subject.

In some embodiments of any of the aspects described herein, the subject is a human subject. In some embodiments of any of the aspects described herein, the subject has or is diagnosed as having cancer.

In one aspect, described herein is a kit for performing any of the assays and/or methods described herein. In some embodiments, the kit can comprise a target-specific reagent.

A kit is any manufacture (e.g., a package or container) comprising at least one reagent, e.g., an antibody reagent(s) or nucleic acid probe, for specifically detecting, e.g., an expression product or fragment thereof of a gene as described herein, the manufacture being promoted, distributed, or sold as a unit for performing the methods or assays described herein. When the kits, and methods described herein are used for diagnosis and/or treatment of cancer in patients, the reagents (e.g., detection probes) or systems can be selected such that a positive result is obtained in at least about 20%, at least about 40%, at least about 60%, at least about 80%, at least about 90%, at least about 95%, at least about 99% or in 100% of subjects having or developing a sensitivity to the therapeutics described herein.

In some embodiments, described herein is a kit for the detection of an expression product in a sample, the kit comprising at least a first target-specific reagent as described herein which specifically binds the expression product, on a solid support and comprising a detectable label. The kits described herein include reagents and/or components that permit assaying the level of an expression product in a sample obtained from a subject (e.g., a biological sample obtained from a subject). The kits described herein can optionally comprise additional components useful for performing the methods and assays described herein.

A kit can further comprise devices and/or reagents for concentrating an expression product (e.g, a polypeptide) in a sample, e.g. a tumor sample. Thus, ultrafiltration devices permitting, e.g., protein concentration from plasma can also be included as a kit component.

Preferably, a diagnostic or prognostic kit for use with the methods and assays disclosed herein contains detection reagents for expression products of targets described herein. Such detection reagents comprise in addition to target-specific reagents, for example, buffer solutions, labels or washing liquids etc. Furthermore, the kit can comprise an amount of a known nucleic acid and/or polypeptide, which can be used for a calibration of the kit or as an internal control. A diagnostic kit for the detection of an expression product can also comprise accessory ingredients like secondary affinity ligands, e.g., secondary antibodies, detection dyes and any other suitable compound or liquid necessary for the performance of a expression product detection method known to the person skilled in the art. Such ingredients are known to the person skilled in the art and may vary depending on the detection method carried out. Additionally, the kit may comprise an instruction leaflet and/or may provide information as to the relevance of the obtained results.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female.

A subject can be one who has been previously diagnosed with or identified as suffering from or having a condition in need of treatment (e.g. cancer) or one or more complications related to such a condition, and optionally, have already undergone treatment for cancer or the one or more complications related to cancer. Alternatively, a subject can also be one who has not been previously diagnosed as having cancer or one or more complications related to cancer. For example, a subject can be one who exhibits one or more risk factors for cancer or one or more complications related to cancer or a subject who does not exhibit risk factors.

A “subject in need” of treatment for a particular condition can be a subject having that condition, diagnosed as having that condition, or at risk of developing that condition.

As used herein the term “chemotherapeutic agent” refers to any chemical or biological agent with therapeutic usefulness in the treatment of diseases characterized by abnormal cell growth. Such diseases include tumors, neoplasms and cancer as well as diseases characterized by hyperplastic growth. These agents can function to inhibit a cellular activity upon which the cancer cell depends for continued proliferation. In some aspect of all the embodiments, a chemotherapeutic agent is a cell cycle inhibitor or a cell division inhibitor. Categories of chemotherapeutic agents that are useful in the methods of the invention include alkylating/alkaloid agents, antimetabolites, hormones or hormone analogs, and miscellaneous antineoplastic drugs. Most of these agents are directly or indirectly toxic to cancer cells. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed. 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). The term is intended to include radioactive isotopes (e.g. At211, 1131, 1125, Y90, Re186, Re188, Sm153, Bi212, P32 and radioactive isotopes of Lu), chemotherapeutic agents, and toxins, such as small molecule toxins or enzymatically active toxins of bacterial, fungal, plant or animal origin, including fragments and/or variants thereof. In some embodiments, the chemotherapeutic agent can be a cytotoxic chemotherapeutic.

As used herein, the term “cancer” relates generally to a class of diseases or conditions in which abnormal cells divide without control and can invade nearby tissues. Cancer cells can also spread to other parts of the body through the blood and lymph systems.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

A subject that has a cancer or a tumor is a subject having objectively measurable cancer cells present in the subject's body. Included in this definition are malignant, actively proliferative cancers, as well as potentially dormant tumors or micrometastatses. Cancers which migrate from their original location and seed other vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice. See, e.g., Freshney, CULTURE ANIMAL CELLS: MANUAL BASIC TECH. (3rd ed., 1994).

As used herein, “engineered” refers to the aspect of having been manipulated by the hand of man. For example, a YAP polypeptide is considered to be “engineered” when the sequence of the polypeptide and/or encoding nucleic acid sequence manipulated by the hand of man to differ from the sequence of an polypeptide as it exists in nature. As is common practice and is understood by those in the art, progeny and copies of an engineered polynucleotide and/or polypeptide are typically still referred to as “engineered” even though the actual manipulation was performed on a prior entity.

As used herein, “recombinant” refers to a cell, tissue or organism that has undergone transformation with a new combination of genes or DNA. When used in reference to nucleic acid molecules, “recombinant” refers to a combination of nucleic acid molecules that are joined together using recombinant DNA technology into a progeny nucleic acid molecule, and/or a heterologous nucleic acid sequence introduced into a cell, tissue, or organism. When used in reference to a polypeptide, “recombinant” refers to a polypeptide which is the expression product of a recombinant nucleic acid, and can be such a polypeptide as produced by a recombinant cell, tissue, or organisms. The nucleic acid molecule can be stably integrated into the genome of the host or the nucleic acid molecule can also be present as an extrachromosomal molecule. Such an extrachromosomal molecule can be auto-replicating. Recombinant viruses, cells, and organisms are understood to encompass not only the end product of a transformation process, but also recombinant progeny thereof.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, a particular “polypeptide”, e.g. a YAP polypeptide can include the human polypeptide (e.g., SEQ ID NO: 12); as well as homologs from other species, including but not limited to bovine, dog, cat chicken, murine, rat, porcine, ovine, turkey, horse, fish, baboon and other primates. The terms also refer to fragments or variants of the native polypeptide that maintain at least 50% of the activity or effect of the native full length polypeptide, e.g. as measured in an appropriate animal model. Conservative substitution variants that maintain the activity of wildtype polypeptides will include a conservative substitution as defined herein. The identification of amino acids most likely to be tolerant of conservative substitution while maintaining at least 50% of the activity of the wildtype is guided by, for example, sequence alignment with homologs or paralogs from other species. Amino acids that are identical between homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants can be tested for activity, for example, by administering the variant to an appropriate animal model of cancer as described herein.

In some embodiments, a polypeptide, e.g., an YAP polypeptide, can be a variant of a sequence described herein, e.g. a variant of an YAP polypeptide comprising the amino acid sequence of SEQ ID NO: 12. In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., at least 50% of the wildtype reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wildtype, e.g. 110%, 125%, 150%, 175%, 200%, 500%, 1000% or more.

One method of identifying amino acid residues which can be substituted is to align, for example, the human polypeptide to a homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, e.g. SEQ ID NO: 12 or a nucleic acid encoding that amino acid sequence. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov), with default parameters set.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, a polypeptide, e.g., an YAP polypeptide, administered to a subject can comprise one or more amino acid substitutions or modifications. In some embodiments, the substitutions and/or modifications can prevent or reduce proteolytic degradation and/or prolong half-life of the polypeptide in the subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide, e.g., a YAP polypeptide, as described herein can comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, e.g., a YAP polypeptide, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, an YAP polypeptide as described herein can comprise alternative amino acids. Non-limiting examples of alternative amino acids include D-amino acids, beta-amino acids, homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, e.g. a YAP polypeptide, can be modified, e.g. by addition of a moiety to one or more of the amino acids comprising the peptide. In some embodiments, a polypeptide as described herein can comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g. PEG or albumin.

In some embodiments, the polypeptide administered to the subject (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. In some embodiments, a polypeptide as described herein can be chemically synthesized and mutations can be incorporated as part of the chemical synthesis process.

In some embodiments, a polypeptide, e.g., a YAP polypeptide, as described herein can be formulated as a pharmaceutically acceptable prodrug. As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. ll, 345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), which are incorporated by reference herein in their entireties.

In some embodiments, a polypeptide as described herein can be a pharmaceutically acceptable solvate. The term “solvate” refers to a peptide as described herein in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The peptides of the present invention can be synthesized by using well known methods including recombinant methods and chemical synthesis. Recombinant methods of producing a peptide through the introduction of a vector including nucleic acid encoding the peptide into a suitable host cell is well known in the art, such as is described in Sambrook et al., Molecular Cloning: A Laboratory Manual, 2d Ed, Vols 1 to 8, Cold Spring Harbor, N.Y. (1989); M. W. Pennington and B. M. Dunn, Methods in Molecular Biology: Peptide Synthesis Protocols, Vol 35, Humana Press, Totawa, N.J. (1994), contents of both of which are herein incorporated by reference. Peptides can also be chemically synthesized using methods well known in the art. See for example, Merrifield et al., J. Am. Chem. Soc. 85:2149 (1964); Bodanszky, M., Principles of Peptide Synthesis, Springer-Verlag, New York, N.Y. (1984); Kimmerlin, T. and Seebach, D. J. Pept. Res. 65:229-260 (2005); Nilsson et al., Annu. Rev. Biophys. Biomol. Struct. (2005) 34:91-118; W. C. Chan and P. D. White (Eds.) Fmoc Solid Phase Peptide Synthesis: A Practical Approach, Oxford University Press, Cary, N.C. (2000); N. L. Benoiton, Chemistry of Peptide Synthesis, CRC Press, Boca Raton, Fla. (2005); J. Jones, Amino Acid and Peptide Synthesis, 2^{11d} Ed, Oxford University Press, Cary, N.C. (2002); and P. Lloyd-Williams, F. Albericio, and E. Giralt, Chemical Approaches to the synthesis of peptides and proteins, CRC Press, Boca Raton, Fla. (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

In some embodiments, the technology described herein relates to a nucleic acid encoding a polypeptide (e.g. a YAP polypeptide) as described herein. As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the nucleic acid is DNA. In another aspect, the nucleic acid is RNA. Suitable nucleic acid molecules are DNA, including genomic DNA or cDNA. Other suitable nucleic acid molecules are RNA, including mRNA. The nucleic acid molecule can be naturally occurring, as in genomic DNA, or it may be synthetic, i.e., prepared based up human action, or may be a combination of the two. The nucleic acid molecule can also have certain modification such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

In some embodiments, a nucleic acid encoding a polypeptide as described herein (e.g. a YAP polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

Inhibitors of the expression of a given gene can be an inhibitory nucleic acid. In some embodiments, the inhibitory nucleic acid is an inhibitory RNA (iRNA). As used herein, the term “iRNA” refers to any type of interfering RNA, including but are not limited to RNAi, siRNA, shRNA, endogenous microRNA and artificial microRNA. Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein effects inhibition of the expression and/or activity of a target gene described herein. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-Co-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA as described herein involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments, an inhibitor of a given polypeptide can be an antibody reagent specific for that polypeptide. As used herein an “antibody” refers to IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a target.

As used herein, “expression level” refers to the number of mRNA molecules and/or polypeptide molecules encoded by a given gene that are present in a cell or sample. Expression levels can be increased or decreased relative to a reference level.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-O-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treating cancer, the method comprising administering a chemotherapeutic selected from the group consisting of:
   an antimetabolite; a nucleoside analog; an antifolate; a topoisomerase I inhibitor; a topoisomerase II inhibitor; an anthracycline; a tubulin modulator; a DNA cross-linking agent; a Src family inase inhibitor; and a BCR-Abl kinase inhibitor;
   to a subject having cancer cells determined to have:
      a. a deletion, a truncation or inactivating mutation in FAT4; LATS1; LATS2; STK11; or NF2;
      b. decreased expression of FAT4; LATS1; LATS2; STK11; or NF2 relative to a reference;
      c. increased expression of YAP; CTGF; AREG; AMOTL2; AXL; or BIRC5 relative to a reference;
      d. decreased phosphorylation of YAP relative to a reference; or
      e. increased nuclear localization of YAP relative to a reference.
2. The method of paragraph 1, wherein the antimetabolite or nucleoside analog is selected from the group consisting of:
   gemcitabine; 5-FU; cladribine; cytarabine; tioguanine; mercaptopurine; and clofarabine.
3. The method of paragraph 1, wherein the antifolate is methotrexate.
4. The method of paragraph 1, wherein the topoisomerase I inhibitor is camptothecin, topotecan, or irrenotecan.
5. The method of paragraph 1, wherein the topoisomerase II inhibitor is selected from the group consisting of:
   epirubicin; daunorubicin; doxorubicin; valrubicin; teniposide; etopiside; and mitoxantrone.
6. The method of paragraph 1, wherein the anthracycline is selected from the group consisting of:
   epirubicin; daunorubicin; doxorubicin; and valrubicin.
7. The method of paragraph 1, wherein the tubulin modulator is ixabepilone.
8. The method of paragraph 1, wherein the Src family kinase inhibitor or BCR-Abl kinase inhibitor is imatinib.
9. The method of paragraph 1, wherein the DNA cross-linking agent is mitomycin.
10. A method of treating cancer, the method comprising administering a chemotherapeutic selected from the group consisting of:
   an antimetabolite; an anthracylcine; an anthracycline topoisomerase II inhibitor; a proteasome inhibitor; an mTOR inhibitor; an RNA synthesis inhibitor; a peptide synthesis inhibitor; an alkylating agent; an antiandrogen; a Src family kinase inhibitor; a BCR-Abl kinase inhibitor; a MEK inhibitor; and a kinase inhibitor;
   to a subject having cancer cells determined not to have:
      a. a deletion, a truncation, or inactivating mutation in FAT4; LATS1; LATS2; STK11; or NF2;
      b. decreased expression of FAT4; LATS1; LATS2; STK11; or NF2 relative to a reference;
      c. increased expression of YAP; CTGF; AREG; AMOTL2; AXL; or BIRC5 relative to a reference;
      d. decreased phosphorylation of YAP relative to a reference; or
      e. increased nuclear localization of YAP relative to a reference.
11. The method of paragraph 10, wherein the anthracycline toposisomerase II inhibitor is selected from the group consisting of:
   daunorubicin; doxorubicin; epirubicin; and valrubicin.
12. The method of paragraph 10, wherein the anthracycline is selected from the group consisting of:
   daunorubicin; doxorubicin; epirubicin; and valrubicin.
13. The method of paragraph 10, wherein the proteasome inhibitor is carfilzomib or bortezomib.
14. The method of paragraph 10, wherein the mTOR inhibitor is everolimus.
15. The method of paragraph 10, wherein the RNA synthesis inhibitor is triethylenemelamine, dactinomycin, or plicamycin.
16. The method of paragraph 10, wherein the kinase inhibitor is ponatinib or trametinib.
17. The method of paragraph 10, wherein the Src family kinase inhibitor or BCR-Abl kinase inhibitor is ponatinib.
18. The method of paragraph 10, wherein the MEK inhibitor is trametinib.
19. The method of paragraph 10, wherein the antiandrogen is enzalutamide.
20. The method of paragraph 10, wherein the peptide synthesis inhibitor is omacetaxine mepesuccinate.
21. The method of any of paragraphs 1-20, wherein the mutation in FAT4; LATS1; LATS2; STK11; or NF2 is selected from Table 2.
22. The method of any of paragraphs 1-21, wherein the method further comprises a step of detecting the presence of one or more of:
   a. a deletion, a truncation, or inactivating mutation in FAT4; LATS1; LATS2; STK11; or NF2;
   b. decreased expression of FAT4; LATS1; LATS2; STK11; or NF2 relative to a reference;
   c. increased expression of YAP; CTGF; AREG; AMOTL2; AXL; or BIRC5 relative to a reference;
   d. decreased phosphorylation of YAP relative to a reference; or
   e. increased nuclear localization of YAP relative to a reference.
23. A method of treating cancer, the method comprising administering
   a. a chemotherapeutic selected from the group consisting of:
      an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; and
   b. an inhibitor of FAT4; STK11; LATS1; LATS2; or NF2; or an agonist of YAP.
24. The method of paragraph 23, wherein the antimetabolite or nucleoside analog is selected from the group consisting of:
   gemcitabine; 5-FU; cladribine; cytarabine; tioguanine; mercaptopurine; and clofarabine.
25. The method of paragraph 23, wherein the antifolate is methotrexate.
26. The method of paragraph 23, wherein the topoisomerase I inhibitor is camptothecin, topotecan, or irrenotecan.
27. The method of paragraph 23, wherein the topoisomerase II inhibitor is selected from the group consisting of:
   epirubicin; daunorubicin; doxorubicin; valrubicin; teniposide; etopiside; and mitoxantrone.
28. The method of paragraph 23, wherein the anthracycline is selected from the group consisting of:
   epirubicin; daunorubicin; doxorubicin; and valrubicin.
29. The method of paragraph 23, wherein the tubulin modulator is ixabepilone.
30. The method of paragraph 23, wherein the Src family kinase inhibitor or BCR-Abl kinase inhibitor is imatinib.
31. The method of paragraph 23, wherein the DNA cross-linking agent is mitomycin.
32. The method of any of paragraphs 23-31, wherein the agonist of YAP is a non-phospho, active form of YAP (e.g. one or more of S61A, S109A, S127A, S128A, S131A, S163A, S164A, S381A mutants) or a nucleic acid encoding a non-phospho, active form of YAP.
33. The method of any of paragraphs 23-31, wherein the inhibitor of FAT4; STK11; LATS1; LATS2; or NF2 is an inhibitory nucleic acid.
34. The method of any of paragraphs 23-31, wherein the inhibitor of STK11 is AZ-23.
35. The method of any of paragraphs 23-31, wherein the inhibitor of LATS2 is GSK690693; AT7867; or PF-477736.
36. The method of any of paragraphs 1-35, wherein the cancer is pancreatic cancer; pancreatic ductal adenocarcinoma; metastatic breast cancer; breast cancer; bladder cancer; small cell lung cancer; lung cancer; ovarian cancer; stomach cancer; uterine cancer; mesothelioma; adenoid cystic carcinoma; lymphoid neoplasm; kidney cancer; colorectal cancer; adenoid cystic carcinoma; prostate cancer; cervical cancer; head and neck cancer; and glioblastoma.
37. An assay comprising:
   detecting, in a test sample obtained from a subject in need of treatment for cancer;
   i. a deletion, a truncation or inactivating mutation in FAT4; LATS1; LATS2; STK11; or NF2;
   ii. decreased expression of FAT4; LATS1; LATS2; STK11; or NF2 relative to a reference;
   iii. increased expression of YAP; CTGF; AREG; AMOTL2; AXL; or BIRC5 relative to a reference;
   iv. decreased phosphorylation of YAP relative to a reference; or
   v. increased nuclear localization of YAP relative to a reference.
   wherein the presence of any of i.-v. indicates the subject is more likely to respond to treatment with a chemotherapeutic selected from the group consisting of:
   an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor.
38. The assay of paragraph 24, wherein the absence of i.-v. indicates the subject should receive treatment with a treatment selected from the group consisting of:
   an antimetabolite; an anthracylcine; an anthracycline topoisomerase II inhibitor; a proteasome inhibitor; an mTOR inhibitor; an RNA synthesis inhibitor; a peptide synthesis inhibitor; an alkylating agent; an antiandrogen; a Src family kinase inhibitor; a BCR-Abl kinase inhibitor; a MEK inhibitor; and a kinase inhibitor;
39. The assay of paragraph 37, wherein the antimetabolite or nucleoside analog is selected from the group consisting of:
   gemcitabine; 5-FU; cladribine; cytarabine; tioguanine; mercaptopurine; and clofarabine.
40. The assay of paragraph 37, wherein the antifolate is methotrexate.
41. The assay of paragraph 37, wherein the topoisomerase I inhibitor is camptothecin, topotecan, or irrenotecan.
42. The assay of paragraph 37, wherein the topoisomerase II inhibitor is selected from the group consisting of:
   epirubicin; daunorubicin; doxorubicin; valrubicin; teniposide; etopiside; and mitoxantrone.
43. The assay of paragraph 37, wherein the anthracycline is selected from the group consisting of:
   epirubicin; daunorubicin; doxorubicin; and valrubicin.
44. The assay of paragraph 37, wherein the tubulin modulator is ixabepilone.
45. The assay of paragraph 37, wherein the Src family kinase inhibitor or BCR-Abl kinase inhibitor is imatinib.
46. The assay of paragraph 37, wherein the DNA cross-linking agent is mitomycin.
47. The assay of paragraph 38, wherein the anthracycline toposisomerase II inhibitor is selected from the group consisting of:
   daunorubicin; doxorubicin; epirubicin; and valrubicin.
48. The assay of paragraph 38, wherein the anthracycline is selected from the group consisting of:
   daunorubicin; doxorubicin; epirubicin; and valrubicin.
49. The assay of paragraph 38, wherein the proteasome inhibitor is carfilzomib or bortezomib.
50. The assay of paragraph 38, wherein the mTOR inhibitor is everolimus.
51. The assay of paragraph 38, wherein the RNA synthesis inhibitor is triethylenemelamine, dactinomycin, or plicamycin.
52. The assay of paragraph 38, wherein the kinase inhibitor is ponatinib or trametinib.
53. The assay of paragraph 38, wherein the Src family kinase inhibitor or BCR-Abl kinase inhibitor is ponatinib.
54. The assay of paragraph 38, wherein the MEK inhibitor is trametinib.
55. The assay of paragraph 38, wherein the antiandrogen is enzalutamide.
56. The assay of paragraph 38, wherein the peptide synthesis inhibitor is omacetaxine mepesuccinate.
57. The assay or method of any of paragraphs 1-56, wherein the determining step comprises measuring the level of a nucleic acid.
58. The assay or method of paragraph 57, wherein the measuring the level of a nucleic acid comprises measuring the level of a RNA transcript.
59. The assay or method of any of paragraphs 57-58, wherein the level of the nucleic acid is determined using a method selected from the group consisting of: RT-PCR; quantitative RT-PCR; Northern blot; microarray based expression analysis; next-generation sequencing; and RNA in situ hybridization.
60. The assay or method of any of paragraphs 1-59, wherein the determining step comprises determining the sequence of a nucleic acid.
61. The assay or method of any of paragraphs 1-59 wherein the determining step comprises measuring the level of a polypeptide.
62. The assay or method of paragraph 61, wherein the polypeptide level is measured using immunochemistry.
63. The assay or method of paragraph 62, wherein the immunochemistry comprises the use of an antibody reagent which is detectably labeled or generates a detectable signal.
64. The assay or method of paragraph 61-63, wherein the level of the polypeptide is determined using a method selected from the group consisting of:
   Western blot; immunoprecipitation; enzyme-linked immunosorbent assay (ELISA);
   radioimmunological assay (RIA); sandwich assay; fluorescence in situ hybridization (FISH); immunohistological staining; radioimmunometric assay; immunofluoresence assay; mass spectroscopy; FACS; and immunoelectrophoresis assay.
65. The assay or method of any of paragraphs 1-64, wherein the expression level is normalized relative to the expression level of one or more reference genes or reference proteins.
66. The assay or method of any of paragraphs 1-65, wherein the reference level is the expression level in a prior sample obtained from the subject.
67. The assay or method of any of paragraphs 1-66, wherein the sample comprises a biopsy; blood; serum; urine; or plasma.
68. A therapeutically effective amount of a chemotherapeutic selected from the group consisting of:
   an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor;
   for use in a method of treating cancer, the method comprising administering the cytotoxic chemotherapeutic to a subject having cancer cells determined to have:
      a. a deletion, a truncation or inactivating mutation in FAT4; LATS1; LATS2; STK11; or NF2;
      b. decreased expression of FAT4; LATS1; LATS2; STK11; or NF2 relative to a reference;
      c. increased expression of YAP; CTGF; AREG; AMOTL2; AXL; or BIRC5 relative to a reference;
      d. decreased phosphorylation of YAP relative to a reference; or
      e. increased nuclear localization of YAP relative to a reference.
69. The use of paragraph 68, wherein the antimetabolite or nucleoside analog is selected from the group consisting of:
   gemcitabine; 5-FU; cladribine; cytarabine; tioguanine; mercaptopurine; and clofarabine.
70. The use of paragraph 68, wherein the antifolate is methotrexate.
71. The use of paragraph 68, wherein the topoisomerase I inhibitor is camptothecin, topotecan, or irrenotecan.
72. The use of paragraph 68, wherein the topoisomerase II inhibitor is selected from the group consisting of:
   epirubicin; daunorubicin; doxorubicin; valrubicin; teniposide; etopiside; and mitoxantrone.
73. The use of paragraph 68, wherein the anthracycline is selected from the group consisting of:
   epirubicin; daunorubicin; doxorubicin; and valrubicin.
74. The use of paragraph 68, wherein the tubulin modulator is ixabepilone.
75. The use of paragraph 68, wherein the Src family kinase inhibitor or BCR-Abl kinase inhibitor is imatinib.
76. The use of paragraph 68, wherein the DNA cross-linking agent is mitomycin.
77. A therapeutically effective amount of a compound selected from the group consisting of:
   an antimetabolite; an anthracylcine; an anthracycline topoisomerase II inhibitor; a proteasome inhibitor; an mTOR inhibitor; an RNA synthesis inhibitor; a peptide synthesis inhibitor; an alkylating agent; an antiandrogen; a Src family kinase inhibitor; a BCR-Abl kinase inhibitor; a MEK inhibitor; and a kinase inhibitor;
   for use in a method of treating cancer, the method comprising administering the compound to a subject having cancer cells determined not to have:
      a. a deletion, a truncation, or inactivating mutation in FAT4; LATS1; LATS2; STK11; or NF2;
      b. decreased expression of FAT4; LATS1; LATS2; STK11; or NF2 relative to a reference;
      c. increased expression of YAP; CTGF; AREG; AMOTL2; AXL; or BIRC5 relative to a reference;
      d. decreased phosphorylation of YAP relative to a reference; or
      e. increased nuclear localization of YAP relative to a reference.
78. The use of paragraph 77, wherein the anthracycline toposisomerase II inhibitor is selected from the group consisting of:
   daunorubicin; doxorubicin; epirubicin; and valrubicin.
79. The use of paragraph 77, wherein the anthracycline is selected from the group consisting of:
   daunorubicin; doxorubicin; epirubicin; and valrubicin.
80. The use of paragraph 77, wherein the proteasome inhibitor is carfilzomib or bortezomib.
81. The use of paragraph 77, wherein the mTOR inhibitor is everolimus.
82. The use of paragraph 77, wherein the RNA synthesis inhibitor is triethylenemelamine, dactinomycin, or plicamycin.
83. The use of paragraph 77, wherein the kinase inhibitor is ponatinib or trametinib.
84. The use of paragraph 77, wherein the Src family kinase inhibitor or BCR-Abl kinase inhibitor is ponatinib.
85. The use of paragraph 77, wherein the MEK inhibitor is trametinib.
86. The use of paragraph 77, wherein the antiandrogen is enzalutamide.
87. The use of paragraph 77, wherein the peptide synthesis inhibitor is omacetaxine mepesuccinate.
88. The use of any of paragraphs 68-87, wherein the mutation in FAT4; LATS1; LATS2; STK11; or NF2 is selected from Table 2.
89. The use of any of paragraphs 68-88, wherein the method further comprises a step of detecting the presence of one or more of:
   a. a deletion, a truncation, or inactivating mutation in FAT4; LATS1; LATS2; STK11; or NF2;
   b. decreased expression of FAT4; LATS1; LATS2; STK11; or NF2 relative to a reference;
   c. increased expression of YAP; CTGF; AREG; AMOTL2; AXL; or BIRC5 relative to a reference;
   d. decreased phosphorylation of YAP relative to a reference; or
   e. increased nuclear localization of YAP relative to a reference.
90. A therapeutically effective amount of a chemotherapeutic selected from the group consisting of:
   an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor; and
   a therapeutically effective amount of an inhibitor of FAT4, STK11, LATS1, LATS2, or NF2, or an agonist of YAP;
   for use in a method of treating cancer, the method comprising administering i) the chemotherapeutic and ii) the inhibitor of FAT4, STK11, LATS1, LATS2, or NF2, or agonist of YAP; to a subject in need of treatment for cancer.
91. The use of paragraph 90, wherein the antimetabolite or nucleoside analog is selected from the group consisting of:
   gemcitabine; 5-FU; cladribine; cytarabine; tioguanine; mercaptopurine; and clofarabine.
92. The use of paragraph 90, wherein the antifolate is methotrexate.
93. The use of paragraph 90, wherein the topoisomerase I inhibitor is camptothecin, topotecan, or irrenotecan.
94. The use of paragraph 90, wherein the topoisomerase II inhibitor is selected from the group consisting of:
   epirubicin; daunorubicin; doxorubicin; valrubicin; teniposide; etopiside; and mitoxantrone.
95. The use of paragraph 90, wherein the anthracycline is selected from the group consisting of:
   epirubicin; daunorubicin; doxorubicin; and valrubicin.
96. The use of paragraph 90, wherein the tubulin modulator is ixabepilone.
97. The use of paragraph 90, wherein the Src family kinase inhibitor or BCR-Abl kinase inhibitor is imatinib.
98. The use of paragraph 90, wherein the DNA cross-linking agent is mitomycin.
99. The use of any of paragraphs 90-98, wherein the agonist of YAP is a non-phospho, active form of YAP (e.g. one or more of S61A, S109A, S127A, S128A, S131A, S163A, S164A, S381A mutants) or a nucleic acid encoding a non-phospho, active form of YAP.
100. The use of any of paragraphs 90-98, wherein the inhibitor of FAT4; STK11; LATS1; LATS2; or NF2 is an inhibitory nucleic acid.
101. The use of any of paragraphs 90-98, wherein the inhibitor of STK11 is AZ-23.
102. The use of any of paragraphs 90-98, wherein the inhibitor of LATS2 is GSK690693; AT7867; or PF-477736.
103. The use of any of paragraphs 68-102, wherein the cancer is pancreatic cancer; pancreatic ductal adenocarcinoma; metastatic breast cancer; breast cancer; bladder cancer; small cell lung cancer; lung cancer; ovarian cancer; stomach cancer; uterine cancer; mesothelioma; adenoid cystic carcinoma; lymphoid neoplasm; kidney cancer; colorectal cancer; adenoid cystic carcinoma; prostate cancer; cervical cancer; head and neck cancer; and glioblastoma.

### EXAMPLES

### Example 1

Described herein is the discovery of a novel role of Hippo-YAP signaling pathway in mediating sensitivity to variety of cytotoxic drugs including gemcitabine. Genetic perturbations reveal de-phosphorylation and nuclear localization of YAP (a hallmark of Hippo pathway) regulates expression of various multidrug transporters, and drug-metabolizing enzyme (cytidine deaminase) thereby increasing the effective cellular drug availability. It is demonstrated herein that cancer cell lines harboring genetic aberrations (deletion or inactivating mutations) in FAT4, LATS2, STK11, and NF2 are extremely sensitive to gemcitabine in both 2D and 3D spheroid assays. Moreover, pancreatic cancer patients (where gemcitabine is a first-line of therapy) with low expression of NF2 or STK11 or high expression of YAP downstream gene signature had prolonged overall survival. Hippo pathway aberrations are found in several cancers where gemcitabine is not a standard-of-care. It is demonstrated herein that alterations in Hippo pathway genes and/or sub-cellular localization of YAP can be used as predictive biomarkers for selection of patients who are likely to respond to gemcitabine. Further, targeting Hippo-YAP pathway can permit treatments to overcome intrinsic drug resistance to gemcitabine in pancreatic cancer.

Pancreatic ductal adenocarcinoma (PDAC) is one of the most lethal forms of cancer. The 1- and 5-year survival rates for PDAC are about 10% and 4.6%, respectively, which are the lowest survival rates of all major cancers. Currently, the nucleoside analogue gemcitabine is the first line treatment of locally advanced and metastatic pancreatic cancer. However, most patients (>75%) treated with gemcitabine do not have an objective response to treatment and only a minority obtains stabilization of disease or partial response. Studying the mechanisms that underlie gemcitabine resistance and discovery of agents that increase the tumor sensitivity to gemcitabine, is therefore desirable.

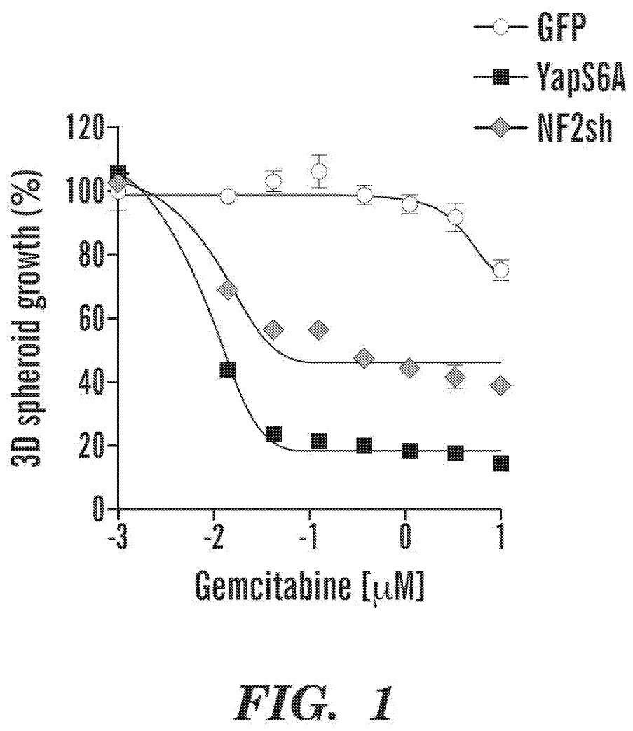

As described herein, the inventors have discovered a novel role of Hippo-YAP signaling pathway in mediating sensitivity to variety of cytotoxic drugs including gemcitabine in PDAC cell lines. All cell lines can be sensitive (IC₅₀<100 nM) or resistant (IC₅₀>1000 nM) to gemcitabine when tested in sparse or dense culture respectively. Cells grown under varying cell-cell contacts (i.e. grown at different densities) differ in many properties including, growth rate, metabolic status, and cell size. Increases in phosphorylation of YAP in density-dependent manner, consistent with previously known role of this pathway in regulating cell density were observed. Phosphorylation of YAP at Ser127 regulates its localization. YAP is localized in the nucleus in cells grown at low density (rapidly dividing) whereas it is retained in the cytosol in the cells grown at high density (growth inhibited). Suppressing hippo pathway by expression of non-phospho, active form of YAP (YAPS6A) or knockdown of NF2 (upstream regulator of YAP phosphorylation) overcomes the contact-dependent inhibition of cell growth and sensitizes pancreatic cancer cells to gemcitabine and other cytotoxic drugs both in 2D and 3D spheroid culture (FIG. 1). Further, it is demonstrated herein that activation of YAP decreases expression of several multidrug transporters including ABCG2, ABCC3 and LRP which reduces cellular efflux of gemcitabine. Thus, a YAP-dependent, combination of increased cell growth and decreased drug efflux renders PDAC cells sensitive to gemcitabine.

Results

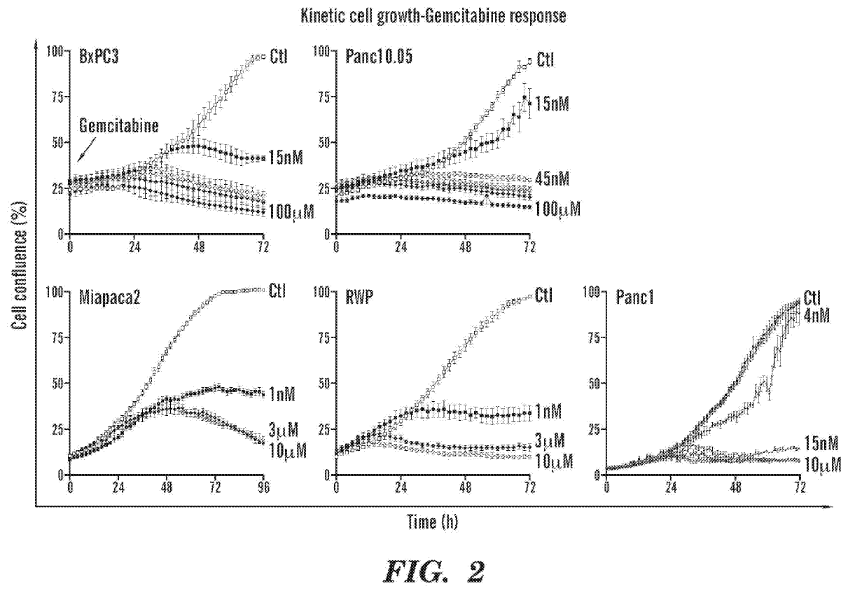

The role of the Hippo pathway in the sensitivity of Panc02.13 cells grown in 3D spheroid to gemcitabine was determined (FIG. 1). Cells were either transfected with GFP vector (GFP), or active form of YAP (YAPS6A) or knockdown of NF2 (NF2sh). “Switching-off” Hippo pathway confers sensitivity to gemcitabine in pancreatic cancer. The effect of gemcitabine on cell growth of five pancreatic cancer cell lines was determined with a live-cell kinetic cell growth assay, characterizing the phenotypic effect of gemcitabine (FIG. 2). Dose response curves were also determined (FIG. 3).

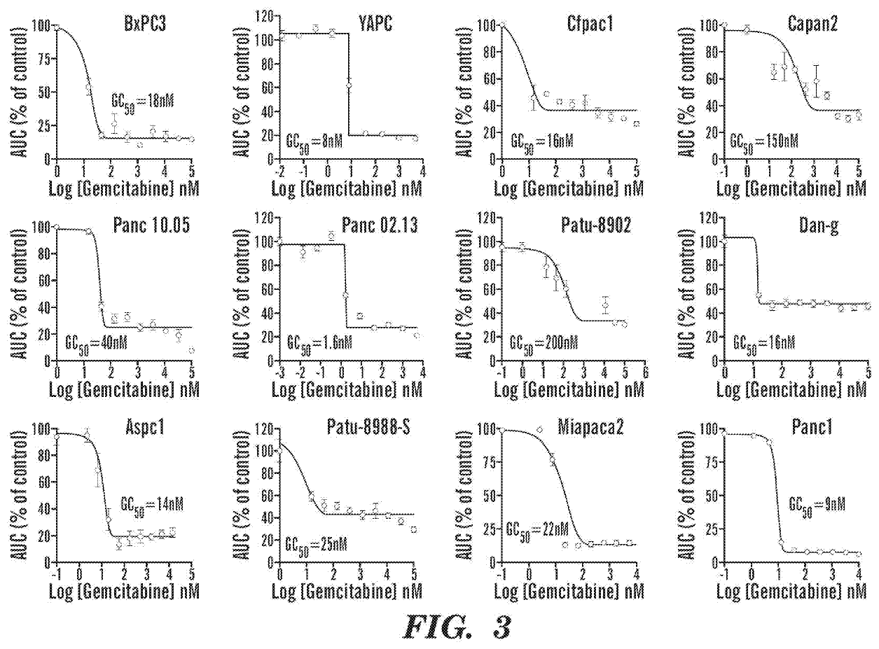

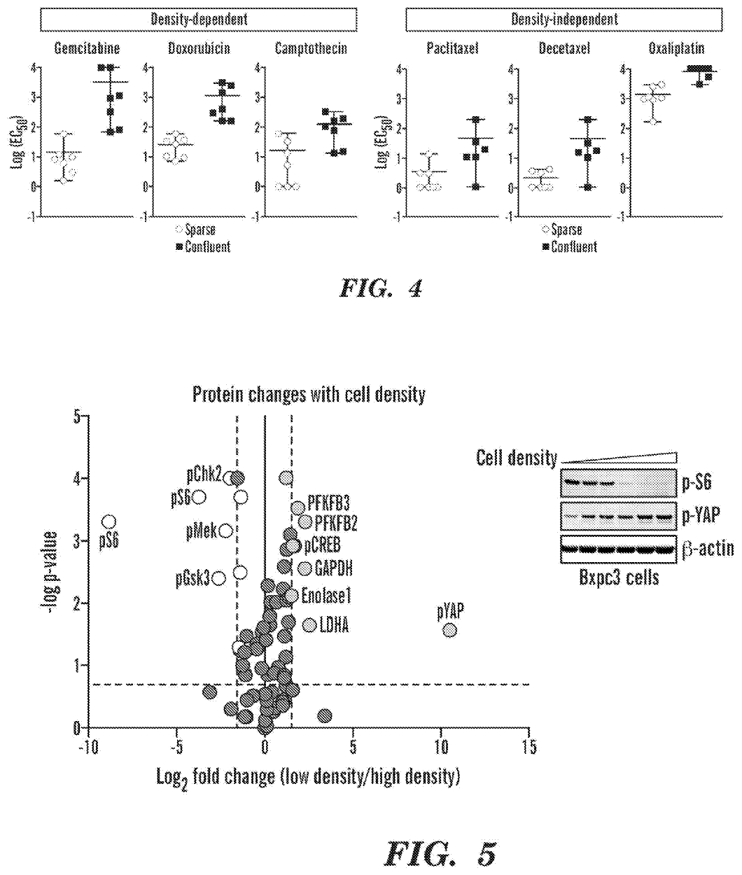

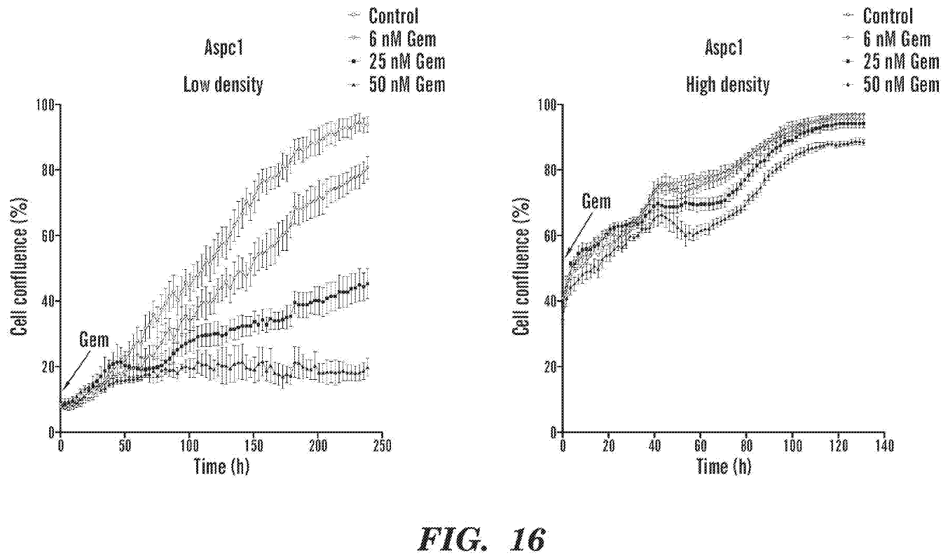

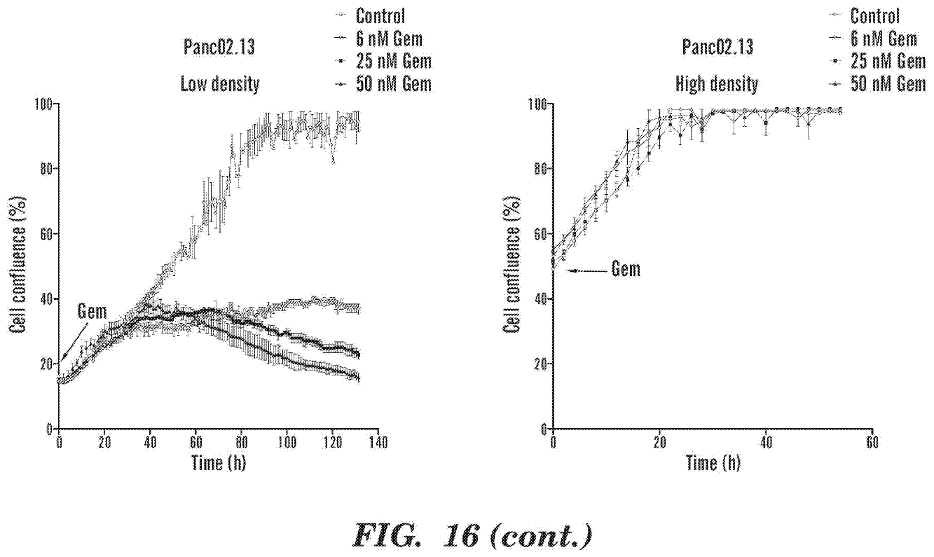

The effect of six cytotoxic drugs on growth of seven pancreatic cancer cell lines under sparse and dense conditions was determined (FIGS. 4 and 16). The efficacy of gemcitabine, doxorubisin and camptothecin was density-dependent while the effects of paclitaxel, Docetaxel and Oxaliplatin were largely density independent.

ASPC1 cells were grown under low or high densities and the protein levels and phosphorylation were determined for each growth condition (FIG. 5). Many growth factor signaling proteins such as Erk, Akt and S6 ribosomal proteins was downregulated when cells are grown in dense cultures. Increase in phosphorylation of YAP in density-dependent manner was also observed. The level of phosphorylation of YAP was also demonstrated to increase as density increased (FIG. 5, right panel).

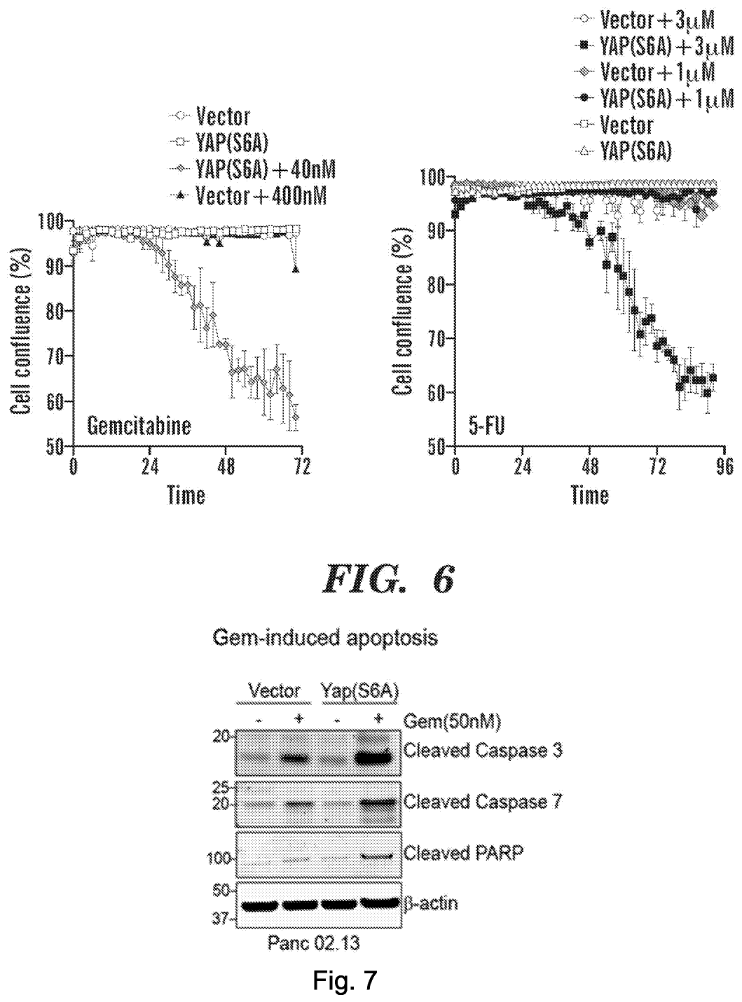

Panc02.13 cells were used to express YAPS6A (or vector controls) under sparse and dense cultures. Expression was confirmed by confocal microscopy (data not shown). Suppression of the Hippo pathway by expression of non-phospho, active form of YAP (YAPS6A) sensitized pancreatic cancer cells to gemcitabine and 5-FU (FIGS. 6 and 7). Apoptosis was measured by immunobloting with cleaved caspases 3/7 or PARP. Blots were also stained with anti-β-actin for loading control. The effect of Hippo pathway suppression on gemcitabine and 5-FU senstitization was maintained in 3D spheroid culture (FIG. 8). The effects of eleven cytotoxic drugs on the growth of Panc02.13 cells expressing vector only or YAPS6A construct grown under low or high densities were determined (Table 1).

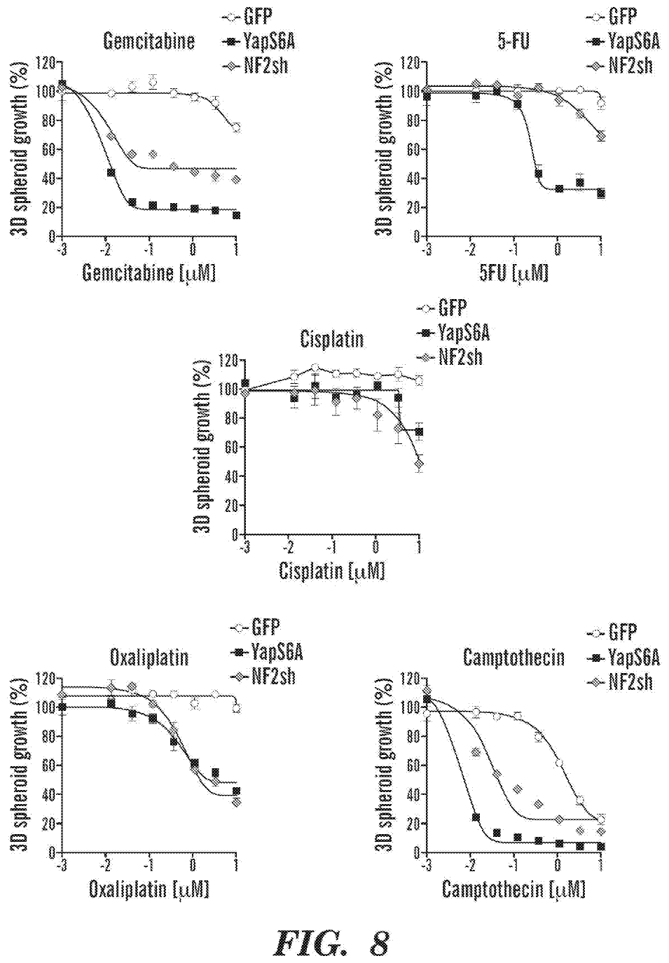

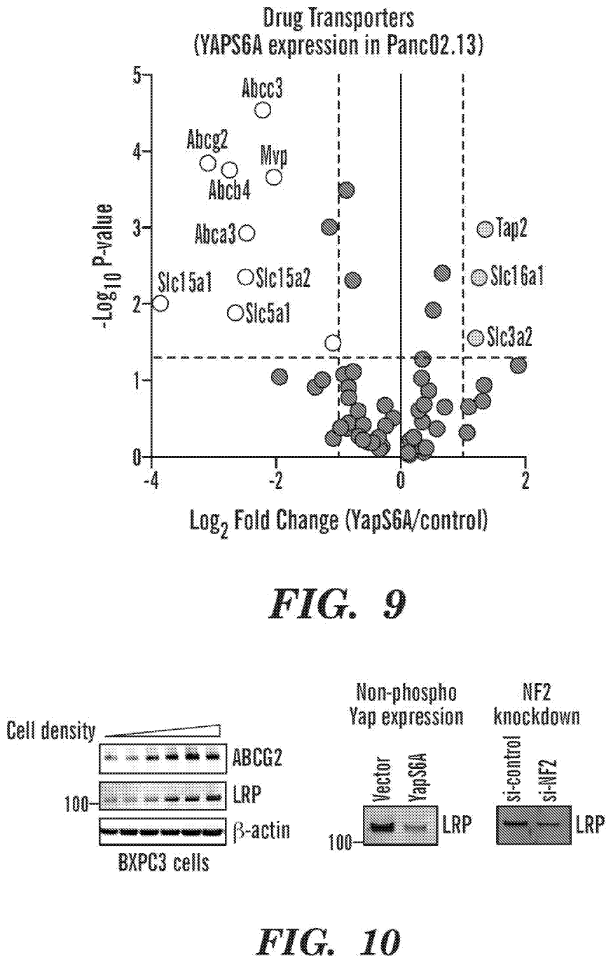

Activation of YAP altered the expression of several multidrug transporters (FIG. 9). mRNA expression profiles for 84 drug transporters in Panc02.13 cells expressing vector control or YAPS6A were determined and, in some cases, confirmed by western blot (FIG. 10). The alteration in drug transport was also evident when gemcitabine efflux (release in the medium) in Panc02.13 cells either grown at low/high densities (left) or with overexpression of YAPS6A (right) was examined (FIG. 11).

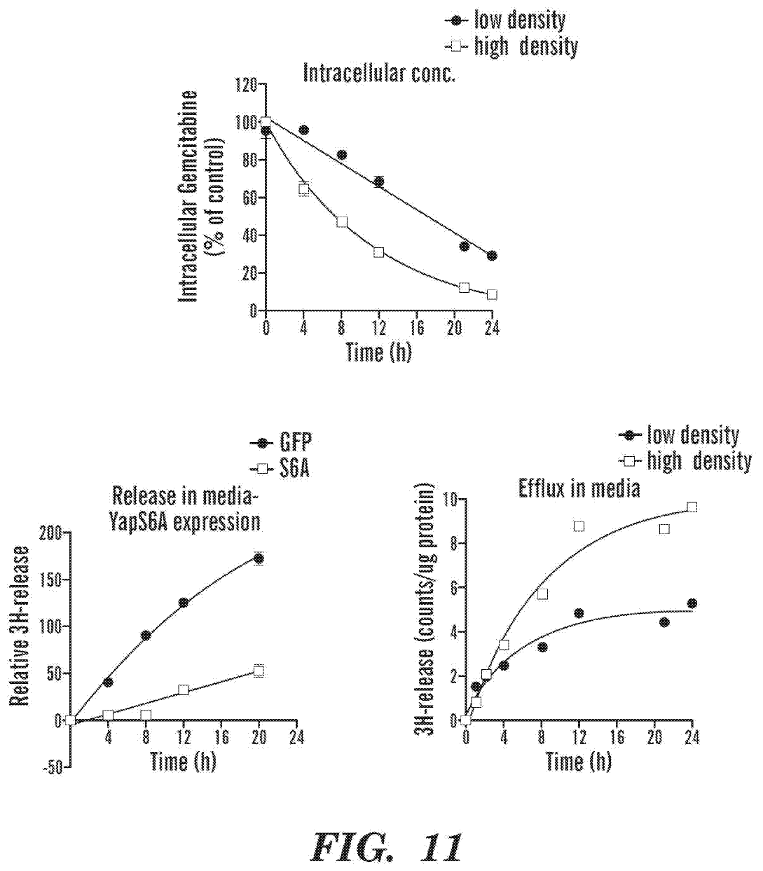

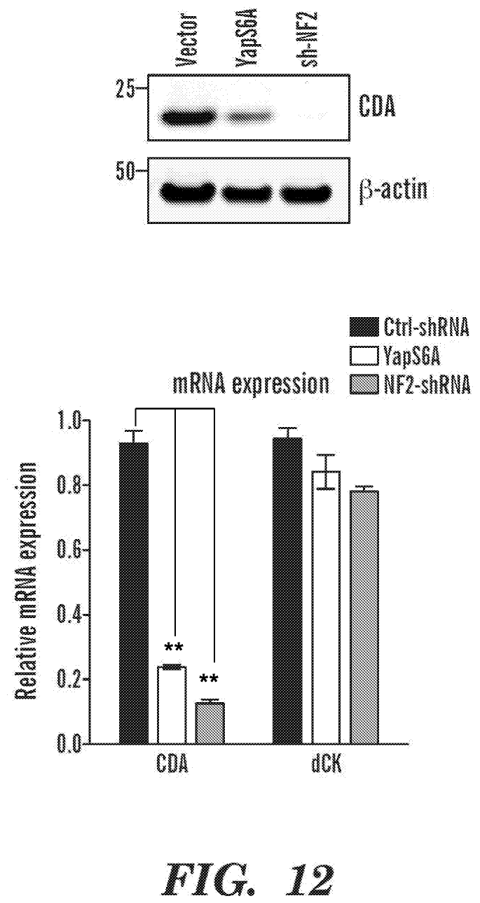

Furthermore, activation of YAP decreases expression of CDA (cytidine deaminase), the key enzyme that metabolizes the drug following its transport into the cell (FIG. 12). Expression of CDA is significantly decreased in Panc02.13 cells expressing, YAPS6A or NF2shRNA compared with vector only control. The mRNA expression of dCK does not change with overexpression of YAPS6A or NF2shRNA.

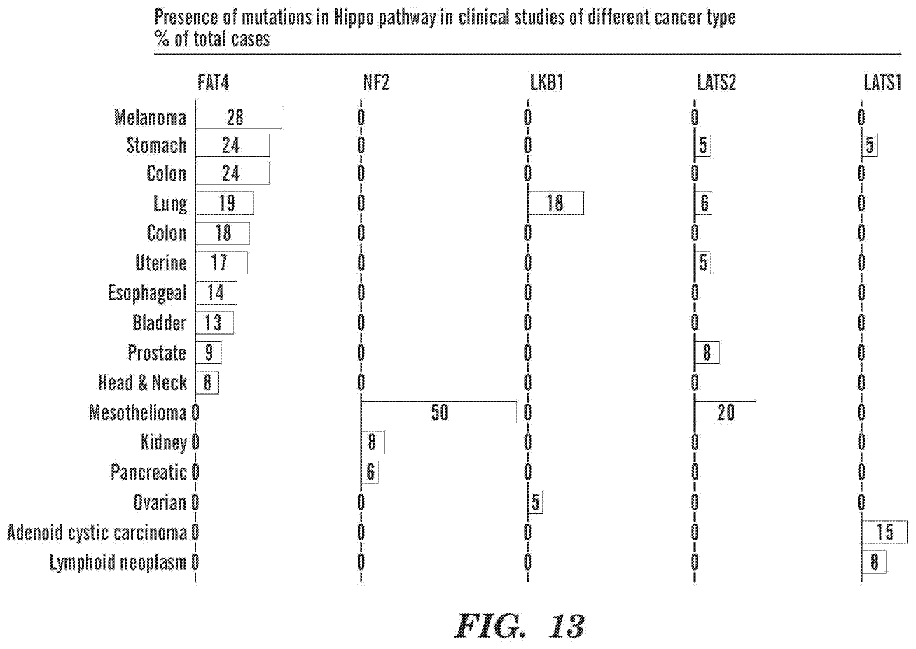

Various cancer types harbor mutations or deletions in the Hippo pathway genes (FIG. 13). Data for this table was compiled using web-based cBioPortal for Cancer Genomics (http://cbioportal.org) pi. Genetic alterations of LATS2 occur in 8% of Prostate cancer (Del, TCGA) 5.5% of Stomach cancer (mut 4.1, del 1.4, TCGA) 5-10% ofUterine cancer (mut, TCGA) and 20% of Mesothelioma. Genetic alterations of LATS1 occur in 15% of Adenoid cyctic carcinoma (del, MSKCC) 9% of Lymphoid neoplasm (del, TCGA) and 4.5% of Stomach cancer (del). Genetic alterations occur in NF2 50% of Mesothelioma 7.4% of Kidney cancer (6.2 del, 1.2 mut, TCGA) and 6% of Pancreatic cancer (del, TCGA) Ovarian, Colorectal, & Gliobalstoma. Genetic alterations in Lkbl (STK11) occur in 21% of Lung cancer (mut) and 5% of ovarian cancer. Amplifications of YAP occur in Cervical cancer (11%), Ovarian (7.4%), Prostate (6%), and H&N (6%).

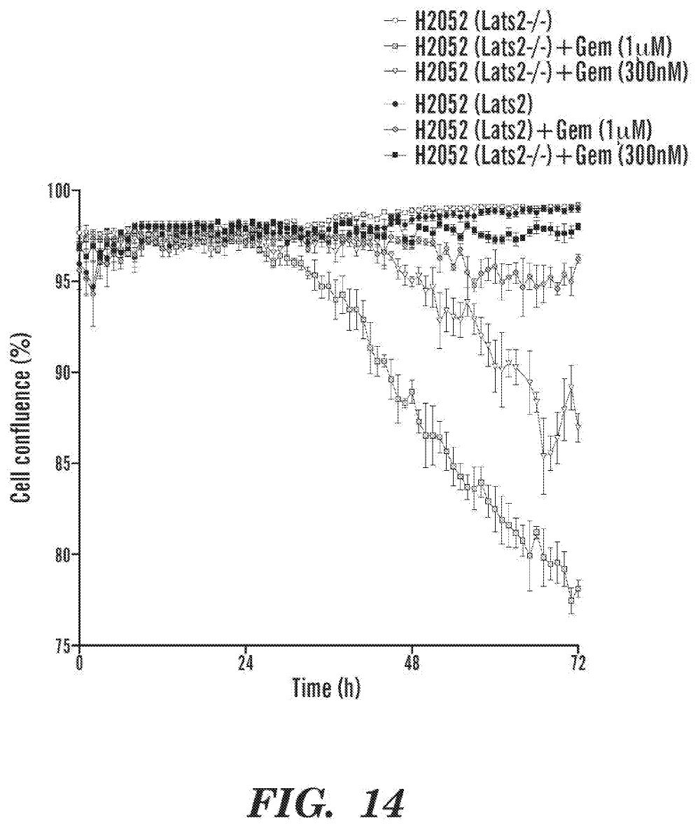

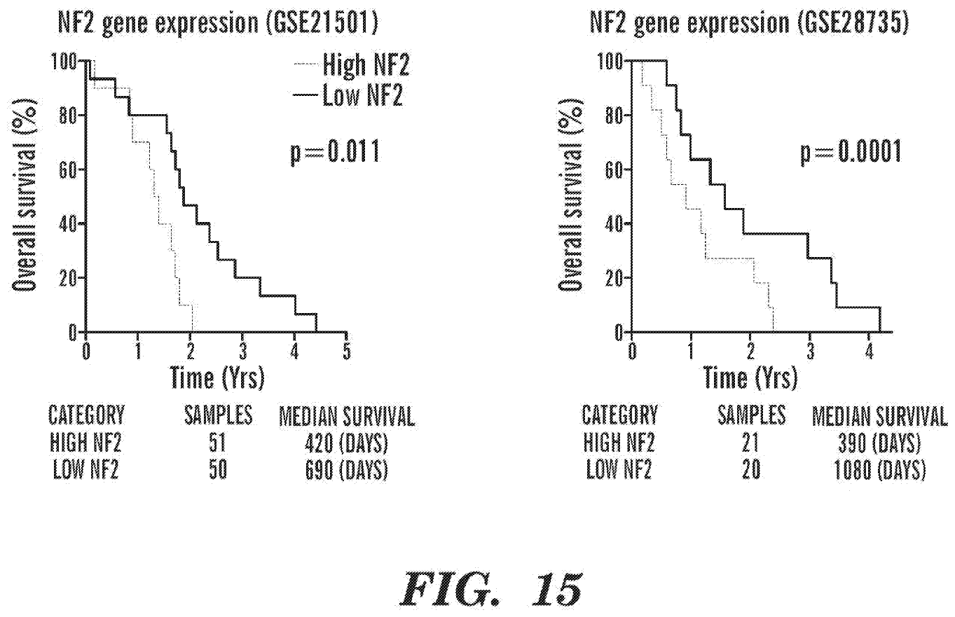

Mesothelioma cells harboring LATS2 deletion are sensitive to gemcitabine and restoring LATS2 expression confers drug resistance (FIG. 14). Low expression of NF2 gene signature is associated with prolong patient survival in pancreatic cancers (FIG. 15).

Materials and Methods

Cell Lines and Reagents.

Pancreatic cancer cell lines Pancl, Panc02.13, BcPC3, Miapaca2, Panc10.05, Capan2, YAPC, CFPAC1, PATU-8902, PATU-8988S, DANG, and ASPC1 cells and mesothelioma cell line H2052 were obtained from American Type Culture Collection (ATCC, Rockville, Md.). Pancl, Miapaca2, PATU-8902, and PATU-8988S were maintained in Dulbecco's Modified Eagle Medium (DMEM) supplemented with 10% (v/v) fetal bovine serum (FBS), 2 mM glutamine, 100 IU/mL penicillin, and 100 μg/mL streptomycin. Panc02.13, BxPC3, Panc10.05, Capan2, YAPC, CFPAC1, DANG, ASPC, and H2052 cells were maintained in Roswell Park Memorial Institute (RPMI) supplemented with 10% (v/v) fetal bovine serum (FBS), 2 mM glutamine, 100 IU/mL penicillin, and 100 μg/mL streptomycin.

Small Molecules.

Gemcitabine hydrochloride (cat # G-4177) was purchased from LC Labs (Woburn, Mass.). Radiolabeled gemcitabine was purchased from American Radiolabeled Chemicals (St. Louis, Mo.). Irrinotecan (cat # S1198), Paclitaxel (cat # S1150), Docetaxel (cat # S1148), Oxaliplatin (cat # S1224), Etoposide (cat # S1225), Camptothecin (cat # S1288) were purchased from Selleckchem (Houston, Tex.).

Antibodies.

Primary antibodies were obtained from the following sources: rabbit phosphor-YAP (S127) (Cell Signaling Technology, Beverly, Mass.; cat. #13008), rabbit anti-YAP (Cell Signaling Technology, Beverly, Mass.; cat. #14074), mouse anti-β-actin (Sigma-Aldrich, Inc., St. Louis, Mo.; cat. # A1978).

Expression Constructs and RNAi.

YAP expression construct with serine-to-alanine mutations at S61A, S109A, S127A, S128A, S131A, S163A, S164A, S381A was purchased from Addgene (Plasmid id: 42562). GIPZ Lentiviral shRNAmir clones for human YAP1 or NF2 were purchased from Dharmacon (Lafeyette, Colo.).

Kinetic Cell Growth Assay.

The effect of gemcitabine on pancreatic cancer cell growth was studied using a kinetic cell growth assay. Pancreatic cancer cells were plated on 96-well plates (Essen ImageLock, Essen Instruments, MI, US) at varying densities (2-4×10³ for low density or 15-20×10³ for high density experiments). Small molecule inhibitors at different doses were added 24 hours after plating and cell confluence was monitored with Incucyte Live-Cell Imaging System and software (Essen Instruments). Confluence was observed every hour for 48-144h or until the control (DMSO only) samples reached 100% confluence.

RNA Extraction and Quantitative Real-Time PCR.

Cells were serum-starved for 24 h and total cellular RNA was isolated using an RNeasy™ Mini Kit (QIAGEN, Santa Clara, Calif.). mRNA levels for the EMT-related genes were determined using the RT² Profiler™ qPCR array (SA Biosciences Corporation, Frederick, Md.). Briefly, 1 μg of total RNA was reverse transcribed into first strand cDNA using an RT² First Strand™ Kit (SA Biosciences). The resulting cDNA was subjected to qPCR using human gene-specific primers for 75 different genes, and five housekeeping genes (B2M, HPRT1, RPL13A, GAPDH, and ACTB). The qPCR reaction was performed with an initial denaturation step of 10 min at 95° C., followed by 15 s at 95° C. and 60 s at 60° C. for 40 cycles using an Mx3000P™ QPCR system (Stratagene, La Jolla, Calif.).

The mRNA levels of each gene were normalized relative to the mean levels of the five housekeeping genes and compared with the data obtained from unstimulated, serum-starved cells using the 2-ΔΔCt method. According to this method, the normalized level of a mRNA, X, is determined using equation 1: (1)

X=2^{-Ct(GOI)}/2^{-Ct(CTL)} (1)

where Ct is the threshold cycle (the number of the cycle at which an increase in reporter fluorescence above a baseline signal is detected), GOI refers to the gene of interest, and CTL refers to a control housekeeping gene. This method assumes that Ct is inversely proportional to the initial concentration of mRNA and that the amount of product doubles with every cycle.

Protein Isolation and Quantitative Western Blotting.

Cells were rinsed in Phosphate Buffered Saline (PBS) and lysed in Lysis Buffer (20 mM Tris-HCl, 150 mM NaCl, 1% Triton X-100 (v/v), 2 mM EDTA, pH 7.8 supplemented with 1 mM sodium orthovanadate, 1 mM phenylmethylsulfonyl fluoride (PMSF), 10 μg/mL aprotinin, and 10 μg/mL leupeptin). Protein concentrations were determined using the BCA protein assay (Pierce, Rockford, Ill.) and immunoblotting experiments were performed using standard procedures. For quantitative immunoblots, primary antibodies were detected with IRDye™ 680-labeled goat-anti-rabbit IgG or IRDye 800-labeled goat-anti-mouse IgG (LI-COR Biosciences, Lincoln, Nebr.) at 1:5000 dilution. Bands were visualized and quantified using an Odyssey™ Infrared Imaging System (LI-COR Biosciences).

Kaplan-Meier Survival Analysis.

Kaplan Meier survival curves of pancreatic cancer patients were generated using PROGgene™ and cBioPortal™, web-based tools [1, 2].

Reverse-Phase Protein Microarray.

Cell lysates prepared from various pancreatic cancer cell lines were printed using Aushon 2470 Arrayer™ (Aushon Biosystems). Validation of antibodies, staining, and analysis of array data was performed as described previously [3].

Generation of YAPS6A Overexpression Cell Lines.

Cell lines (Panc02.13, Panc10.05 or Miapaca2) were transfected with YAPS6A constructs (Addgene) using Lipofectamine™ (Invitrogen, Carlsbad, Calif.) following the manufacturer's instructions and 48 hour post-transfection selected in 5-10 μg/ml Blasticidin (InvivoGen, San Diego, Calif.). The clones screened for YAPS6A expression by Western blot. Stable cell lines were maintained in complete medium and 5 μg/ml Blasticidin.

Confocal Imaging.

Panc02.13 cells were cultured on Lab-Tek II™ chamber glass slides (Nalge Nunc, Naperville, Ill.) or on 24-well glass bottom dishes (MatTek Corporation). Cells were fixed in 4% paraformaldehyde for 15 min at room temperature, washed in PBS, permeabilized with 0.1% Triton X-100, and blocked for 60 min with PBS containing 3% BSA (w/v). Cells were immunostained with the appropriate antibody, following by immunostaining with Alexa Fluor 488-labeled goat-anti-rabbit antibody (Molecular Probes, Eugene, Oreg.). Nuclei were counterstained with Hoescht 33342 (Sigma-Aldrich, St. Louis, Mo.). Fluorescent micrographs were obtained using a Nikon A1R™ point scanning confocal microscope. Individual channels were overlaid using ImageJ™ software (National Institutes of Health, Bethesda, Md.)

3D Spheroid Assay.

Cancer cell lines were seeded at a 5×103 cells per well in a 96-well ultra-low adherence plates (Costar) and briefly spun down at 1000 rpm for 5 minutes. After 2 days, cells were treated with small molecule inhibitors at varying concentrations. Growth of spheroids was monitored using live cell imaging every 2-3 hours for 4-7 days in the Incucyte FLR™ system (Essen) or as end point assay using CellTiter-Glo™ luminescent cell viability assay (Promega).

Measuring Gemcitabine Efflux.

Panc02.13 cells expressing GFP or YapS6A plasmid were treated with radiolabeled gemcitabine (0.5 μM) for one hour. Cells were washed twice with PBS and incubated in fresh medium. Medium was collected over the time course of 24 hours and radioactivity was measured using scintillation counter.

Profiling Drug Transporters.

mRNA expression of drug transporters was profiled using Human Drug transporters PCR Array from SA Biosciences (cat # PAHS-070Z) using manufacturer's instructions.

### REFERENCES

1. Goswami, C. P. and H. Nakshatri, PROGgene: gene expression based survival analysis web application for multiple cancers. J Clin Bioinforma, 2013. 3(1): p. 22.
2. Gao, J., et al., Integrative analysis of complex cancer genomics and clinical profiles using the cBioPortal. Sci Signal, 2013. 6(269): p. p 11.
3. Gujral, T. S., et al., Profiling phospho-signaling networks in breast cancer using reverse phase protein arrays. Oncogene, 2012.

TABLE 1
Table showing the effect of eleven cytotoxic drugs on the growth of Panc02.13
cells expressing vector only or YAPS6A construct grown under low or high densities.
The respective EC₅₀ values in nanomolar for each drug is indicated.

| | | Response | | | |
|---|---|---|---|---|---|
| | | Low density (EC₅₀, nM) | | High density (EC₅₀, nM) | |
| | | 02.13- | 02.13- | 02.13- | 02.13- |
| Class | Drug | WT | YAPS6A | WT | YAPS6A |
| Nucleoside | Gemcitabine | 1.6 | 1.7 | 1100 | 17 |
| analogs | 5-FU | 350 | | >10000 | 3000 |
| Platinum | Cisplatin | >10000 | >10000 | >10000 | >10000 |
| | Oxaliplatin | 937 | 5890 | 5200 | 3184 |
| Topoisomerase | Irrenotecan (Topo I) | 1072 | 1697 | 8500 | 1649 |
| Inhibitors | Camtothecin (Topo I) | 5.2 | 6 | 15 | 9 |
| | Doxorubicin (Topo II) | 35 | 68 | 386 | 133 |
| | Etoposide (Topo II) | 386 | 383 | 2600 | 942 |
| Taxanes | Docetaxel | 3.5 | 7 | 2400 | 4825 |
| | Paclitaxel | 3 | 3 | 1674 | |
| | Epothilone | <1 | <1 | 20 | 767 |

### Example 2

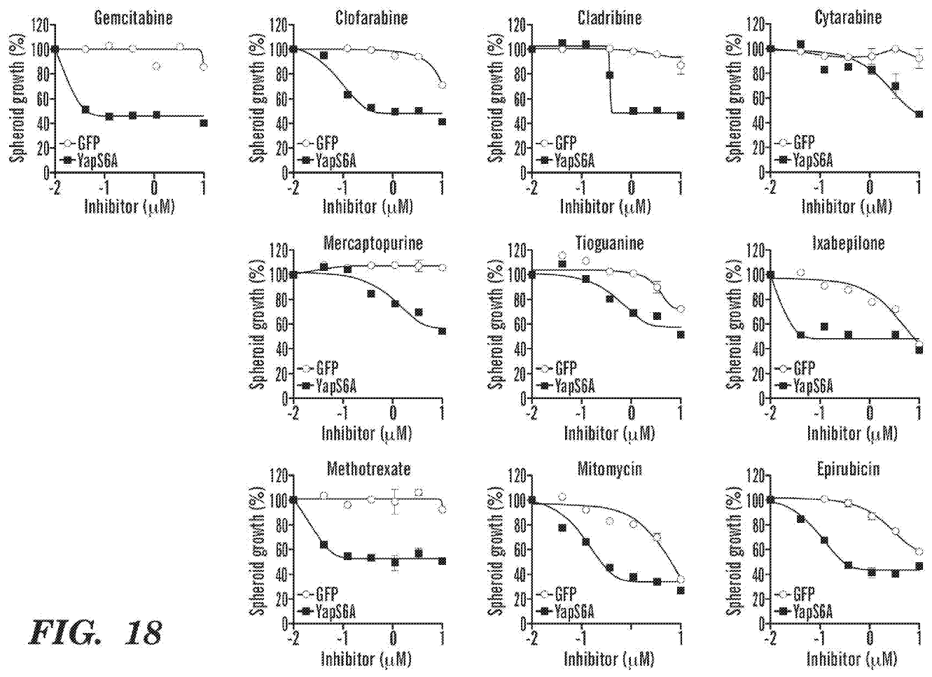

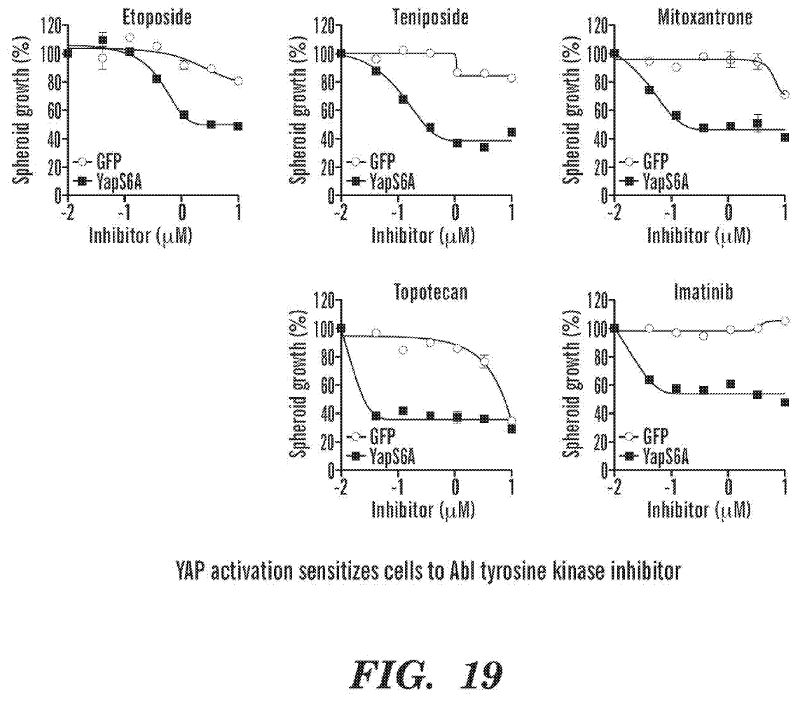

As described herein, a number of compounds were found to have increased efficacy in inhibiting cell growth when the Hippo pathway was inhibited (e.g. YAP activity was increased). Those compounds include: gemcitabine; 5-FU; cladribine; cytarabine; tioguanine; mercaptopurine; clofarabine; methotrexate; camptothecin, topotecan, irrenotecan; epirubicin; daunorubicin; doxorubicin; valrubicin; teniposide; etopiside; mitoxantrone; ixabepilone; imatinib; mitomycin (see, e.g. FIGS. 18 and 19).

Additionally, a number of compunds were demonstrated to be efficacious in inhibiting cell growth when the Hippo pathway was not inhibited. Those compounds include: daunorubicin; doxorubicin; epirubicin; valrubicin; carfilzomib; bortezomib; dactinomycin; plicamycin; ponatinib; trametinib; enzalutamide; and omacetaxine mepesuccinate. Everolimus and triethylenemelamine demonstrated efficacy at higher doses (see, e.g. FIGS. 18 and 19).

TABLE 2

| FAT4 | LKB1 | NF2 | LATS1 | LATS2 |
|---|---|---|---|---|
| 3424_3425TF > I | 137_138QE > H* | A433V | 849_850LC > R | *1089Y |
| A114T | A200_splice | A6V | A161V | 472_480APAPAPAPA > A |
| A114V | A389T | C133Y | A47S | 479_479P > PAP |
| A1259G | A43fs | D245G | A483T | A110T |
| A132T | D176A | D494N | A549S | A120G |
| A1375V | D194H | E103D | A748T | A251T |
| A1534T | D194N | E107* | A805V | A309V |
| A1603E | D237Y | E129* | A810S | A324V |
| A1693T | D23fs | E166D | A899fs | A392fs |
| A1694V | D327fs | E186* | D1043N | A392T |
| A1702T | D350E | E202A | D1086Y | A428V |
| A1711V | D355N | E215* | D837H | A497T |
| A1798T | D358N | E215D | D871Y | A546V |
| A2155V | D359Y | E231* | D994N | A561T |
| A2157V | D53fs | E247* | E100* | A678S |
| A2178P | E120* | E270_splice | E36D | A773T |
| A2178S | E130* | E38_splice | E574* | A861V |
| A2214T | E145* | E386fs | E592K | A881V |
| A2421T | E165* | E392K | E594K | A944V |
| A2525T | E199* | E427* | E606G | A95V |
| A2562T | E223* | E427K | E689* | C1083Y |
| A2604T | E256* | E465* | E802K | D1048N |
| A275S | E265* | E527Q | E920fs | D1078Y |
| A2814S | E265fs | E541* | E920G | D564Y |
| A3073S | E317* | E58* | F1010fs | D569G |
| A3096T | E317K | F162fs | F1015L | D56Y |
| A3113V | E33* | F256fs | F532fs | D800Y |
| A3119V | E357K | F96L | F641L | D852N |
| A3227V | E70* | H195P | G1106A | D956V |
| A3482D | G155_splice | H84Y | G113E | E1016K |
| A3554D | G196V | H95Y | G166_splice | E1039K |
| A3753T | G227fs | I264V | G231* | E1067A |
| A3855V | G242V | K171* | G448W | E130K |
| A389S | G242W | K227fs | G535E | E591fs |
| A389T | G251F | K80_splice | G554E | E652* |
| A4031V | G251R | K99T | G787A | E722* |
| A4149V | G251V | L295fs | G787V | E726K |
| A4165T | G268fs | L297fs | G823V | E765D |
| A4353V | G268R | L505M | G937V | F810S |
| A4481T | G270fs | L558V | H1007Y | F972L |
| A4485P | G279fs | M39_splice | H359L | G218V |
| A4504V | G288_splice | M9T | H417D | G293D |
| A4507V | G56fs | MUTATED | H475Y | G363S |
| A4513T | G56V | N248S | H52Y | G36W |
| A4760T | G56W | P134H | H820R | G40E |
| A488T | G61fs | P155fs | I131M | G498V |
| A4908E | G91L | P170fs | I131V | G539D |
| A4909S | H168R | P246L | I220V | G566C |
| A545T | H174Y | P252H | I288M | G803C |
| A566V | I111N | P275fs | I81M | G851E |
| A661T | I177T | P486fs | K1005* | G92S |
| A673V | I29fs | P488L | K1005N | H222Tfs*18 |
| A777T | I303N | P492S | K607N | H317Y |
| C2437F | I303S | P91L | L109S | H691fs |
| C3279F | K108* | Q111* | L78fs | H970N |
| C3871W | K175fs | Q121_splice | L793Q | I149T |
| C3904F | K191* | Q178* | M310V | I80fs |
| C4692S | K235* | Q333_splice | M419I | I902L |
| C4806Y | K262* | Q389* | M704V | I902N |
| D1095N | K287* | Q459H | M782I | K665R |
| D1128Y | K62* | Q470* | M790T | K702M |
| D1145N | K78E | Q470L | MUTATED | L331M |
| D1202E | K97_splice | R172_splice | N1038H | L625V |
| D1289N | L183V | R196G | N463S | L693M |
| D1310fs | L195M | R198Q | N471S | L699V |
| D1323E | L245_splice | R200_splice | N551S | L77fs |
| D1343N | L245F | R291C | N762S | L841F |
| D1379V | L285Q | R338C | N999D | L903I |
| D1379Y | L50fs | R338H | P1028A | L914fs |
| D137N | L55fs | R341* | P1028T | L967M |
| D1415A | M125_splice | R359fs | P158S | N725S |
| D1485N | M51fs | R418C | P237fs | P166S |
| D1521N | N181I | R424C | P237Q | P190fs |
| D1538fs | N181Y | R466Q | P250S | P190S |
| D1605E | N226K | R57* | P251A | P208L |
| D1790N | N259fs | S10fs | P257fs | P210S |
| D1824Y | P144fs | S12fs | P258S | P305L |
| D1853Y | P179Q | S143F | P266fs | P414L |
| D1868N | P179R | S246F | P292fs | P516L |
| D1883N | P179S | S265L | P292L | P551L |
| D1970N | P221L | S267fs | P301H | P577L |
| D2007Y | P221S | S288* | P301S | P72L |
| D2043Y | P281fs | S444fs | P375S | P86S |
| D2046E | P294L | S87* | P377S | P996L |
| D2128N | P314H | T352M | P434R | Q105P |
| D2149N | P369S | T480M | P445L | Q1079E |
| D2288N | Q112* | V146I | P452H | Q345* |
| D2424V | Q123* | W184R | P468S | Q63del |
| D2429N | Q137* | Y132* | P493S | Q643E |
| D242N | Q159* | Y132C | P506L | Q74R |
| D2542Y | Q170* | Y144* | P506R | R1043* |
| D2563N | Q220* | Y144fs | P531fs | R1054* |
| D2656N | Q37* | Y221C | P568L | R1054Q |
| D2661V | Q37fs | Y528C | P579S | R16L |
| D2664N | Q37L | | Q188R | R18* |
| D2732G | R304G | | Q225E | R271H |
| D3012Y | R310P | | Q273* | R391H |
| D3063H | R331fs | | Q553E | R415W |
| D3153H | R75fs | | Q678H | R525C |
| D3186N | R86Q | | Q863E | R558H |
| D3186Y | S193F | | Q903* | R581C |
| D3387N | S193fs | | R1020T | R593C |
| D3397E | S19P | | R1082K | R623W |
| D3400G | S216F | | R1125C | R645L |
| D3502N | S307_splice | | R1125H | R759W |
| D3505N | T250fs | | R147* | R769W |
| D3588V | T336fs | | R174C | R790Q |
| D3640N | V236A | | R233S | R817G |
| D3642N | W239C | | R252* | R832G |
| D3645N | W332* | | R252I | R849L |
| D3645Y | Y272* | | R287* | R983L |
| D3802N | Y60* | | R28Q | S179L |
| D3804N | Y60fs | | R35L | S33L |
| D3958N | | | R35W | S366F |
| D4021G | | | R502C | S528L |
| D4021V | | | R63Q | S596R |
| D4283N | | | R657C | S872L |
| D4363H | | | R682T | S91L |
| D4429E | | | R694C | T1019I |
| D470N | | | R737* | T1041I |
| D4727N | | | R744* | T1041P |
| D4809Y | | | R744L | T168M |
| D4831H | | | R744Q | T673I |
| D4877N | | | R767L | T876N |
| D4882G | | | R82* | V1086A |
| D4949A | | | R827S | V621L |
| D628N | | | R827T | V682L |
| D785G | | | R82Q | V729D |
| D788A | | | R838G | W842L |
| D876Y | | | R854K | Y183C |
| E1007V | | | R924* | Y506F |
| E1037K | | | R96L | Y531H |
| E1110K | | | R96Q | |
| E1221Q | | | R990Q | |
| E1255K | | | R995H | |
| E1308K | | | R995L | |
| E1381K | | | S1023C | |
| E1431Q | | | S207* | |
| E1566D | | | S216fs | |
| E1566K | | | S278C | |
| E1642* | | | S308F | |
| E1642K | | | S336G | |
| E1699V | | | S387F | |
| E1725D | | | S438F | |
| E1725G | | | S444P | |
| E1875K | | | S45Y | |
| E2061* | | | S792I | |
| E2165K | | | S803T | |
| E2183K | | | T255N | |
| E2201K | | | T367I | |
| E223K | | | T851I | |
| E2315* | | | V1057A | |
| E2653K | | | V234L | |
| E2677Q | | | V25F | |
| E2680K | | | V284I | |
| E2713K | | | W178* | |
| E2724* | | | W268* | |
| E2734K | | | W519C | |
| E2883K | | | Y200S | |
| E292* | | | Y862C | |
| E2926* | | | | |
| E2926D | | | | |
| E301D | | | | |
| E3134* | | | | |
| E3134D | | | | |
| E314K | | | | |
| E3161K | | | | |
| E3293* | | | | |
| E3319* | | | | |
| E3449K | | | | |
| E3449V | | | | |
| E3516D | | | | |
| E3519K | | | | |
| E3618D | | | | |
| E3788K | | | | |
| E3799Q | | | | |
| E3831* | | | | |
| E3982K | | | | |
| E4032K | | | | |
| E4083* | | | | |
| E4374D | | | | |
| E4442D | | | | |
| E4497* | | | | |
| E4497D | | | | |
| E4497K | | | | |
| E4545D | | | | |
| E4552* | | | | |
| E4595K | | | | |
| E4603Q | | | | |
| E4616G | | | | |
| E4618* | | | | |
| E4720K | | | | |
| E4793D | | | | |
| E4858K | | | | |
| E4875K | | | | |
| E4961K | | | | |
| E4962* | | | | |
| E754D | | | | |
| E904D | | | | |
| E922* | | | | |
| E944K | | | | |
| F1015S | | | | |
| F1109L | | | | |
| F1118L | | | | |
| F1175L | | | | |
| F2513I | | | | |
| F2861V | | | | |
| F2989L | | | | |
| F3022L | | | | |
| F3055V | | | | |
| F3056C | | | | |
| F313L | | | | |
| F3338V | | | | |
| F3378V | | | | |
| F3440V | | | | |
| F3558S | | | | |
| F3783L | | | | |
| F4025fs | | | | |
| F4037L | | | | |
| F4250C | | | | |
| F4642L | | | | |
| F4706V | | | | |
| F4743L | | | | |
| F654L | | | | |
| G1050V | | | | |
| G1423A | | | | |
| G1453D | | | | |
| G1561E | | | | |
| G1582* | | | | |
| G1623V | | | | |
| G1645D | | | | |
| G1782R | | | | |
| G1857C | | | | |
| G1921C | | | | |
| G1922E | | | | |
| G195C | | | | |
| G195S | | | | |
| G195V | | | | |
| G1960* | | | | |
| G1960E | | | | |
| G1986C | | | | |
| G1998C | | | | |
| G206R | | | | |
| G207R | | | | |
| G207V | | | | |
| G2170E | | | | |
| G2170V | | | | |
| G2181E | | | | |
| G2209S | | | | |
| G2235fs | | | | |
| G2314E | | | | |
| G2317V | | | | |
| G2340E | | | | |
| G2507C | | | | |
| G2507V | | | | |
| G251D | | | | |
| G2530R | | | | |
| G258W | | | | |
| G2596R | | | | |
| G2606fs | | | | |
| G2749E | | | | |
| G2851E | | | | |
| G2888R | | | | |
| G2902E | | | | |
| G2905E | | | | |
| G3014V | | | | |
| G3065E | | | | |
| G3122D | | | | |
| G3131E | | | | |
| G3135S | | | | |
| G3135V | | | | |
| G3210E | | | | |
| G3254R | | | | |
| G3331W | | | | |
| G3420E | | | | |
| G3445E | | | | |
| G3445R | | | | |
| G3507R | | | | |
| G3507W | | | | |
| G3552C | | | | |
| G3625S | | | | |
| G3631E | | | | |
| G3718S | | | | |
| G3795R | | | | |
| G3853V | | | | |
| G3882E | | | | |
| G3883* | | | | |
| G3929E | | | | |
| G3967_splice | | | | |
| G3979* | | | | |
| G3979R | | | | |
| G4044C | | | | |
| G4057E | | | | |
| G4110R | | | | |
| G4242C | | | | |
| G4285E | | | | |
| G42K | | | | |
| G42W | | | | |
| G4337V | | | | |
| G4361V | | | | |
| G4380W | | | | |
| G4397V | | | | |
| G4439V | | | | |
| G4448S | | | | |
| G4459E | | | | |
| G4476E | | | | |
| G4486W | | | | |
| G4531R | | | | |
| G4681C | | | | |
| G4728D | | | | |
| G4786* | | | | |
| G4786R | | | | |
| G4832A | | | | |
| G4885R | | | | |
| G4895S | | | | |
| G4900E | | | | |
| G4901E | | | | |
| G4922W | | | | |
| G610W | | | | |
| G639E | | | | |
| G704A | | | | |
| G768D | | | | |
| G768V | | | | |
| G813C | | | | |
| G88V | | | | |
| G926D | | | | |
| G947D | | | | |
| H1159N | | | | |
| H2514Y | | | | |
| H3601Y | | | | |
| H3732fs | | | | |
| H3770N | | | | |
| H3803Y | | | | |
| H406Q | | | | |
| H4487Q | | | | |
| H4722R | | | | |
| H697R | | | | |
| H811Y | | | | |
| I1035V | | | | |
| I140M | | | | |
| I1429T | | | | |
| I1505L | | | | |
| I1683T | | | | |
| I1759fs | | | | |
| I1759M | | | | |
| I1779T | | | | |
| I2039T | | | | |
| I2085T | | | | |
| I2153N | | | | |
| I2247T | | | | |
| I2971T | | | | |
| I2973R | | | | |
| I3057M | | | | |
| I3107V | | | | |
| I3337L | | | | |
| I3836N | | | | |
| I420M | | | | |
| I4343V | | | | |
| I4403T | | | | |
| I4605V | | | | |
| I525M | | | | |
| I728M | | | | |
| I830V | | | | |
| K1251E | | | | |
| K1376fs | | | | |
| K1376T | | | | |
| K1809* | | | | |
| K1809I | | | | |
| K1840T | | | | |
| K1996N | | | | |
| K2001R | | | | |
| K2096T | | | | |
| K2395N | | | | |
| K2428Q | | | | |
| K2512I | | | | |
| K2566N | | | | |
| K2758N | | | | |
| K2994T | | | | |
| K2997N | | | | |
| K3313E | | | | |
| K3343R | | | | |
| K3350M | | | | |
| K3350R | | | | |
| K3372E | | | | |
| K4006N | | | | |
| K4274N | | | | |
| K4311fs | | | | |
| K4381N | | | | |
| K453* | | | | |
| K4532N | | | | |
| K4533T | | | | |
| K4549T | | | | |
| K4948R | | | | |
| K945N | | | | |
| KD2428del | | | | |
| L1062R | | | | |
| L111I | | | | |
| L1230F | | | | |
| L1374P | | | | |
| L1455V | | | | |
| L1535I | | | | |
| L1590F | | | | |
| L1621F | | | | |
| L1747F | | | | |
| L1762P | | | | |
| L1813P | | | | |
| L2280F | | | | |
| L2280H | | | | |
| L2280R | | | | |
| L2422F | | | | |
| L2423S | | | | |
| L2446F | | | | |
| L2655R | | | | |
| L2884R | | | | |
| L289P | | | | |
| L2984F | | | | |
| L2984R | | | | |
| L3051V | | | | |
| L3123V | | | | |
| L3146* | | | | |
| L3266F | | | | |
| L3336F | | | | |
| L3361R | | | | |
| L3361V | | | | |
| L3406P | | | | |
| L3468V | | | | |
| L3566M | | | | |
| L3668M | | | | |
| L3762P | | | | |
| L3833I | | | | |
| L3V | | | | |
| L4011F | | | | |
| L4012I | | | | |
| L4048P | | | | |
| L419P | | | | |
| L4469H | | | | |
| L4518F | | | | |
| L4525P | | | | |
| L4888R | | | | |
| L4921I | | | | |
| L510V | | | | |
| L540M | | | | |
| L550V | | | | |
| L634V | | | | |
| L976F | | | | |
| M2333I | | | | |
| M2712K | | | | |
| M3120I | | | | |
| M3162I | | | | |
| M3518T | | | | |
| M4135I | | | | |
| M4369L | | | | |
| M4853T | | | | |
| M820I | | | | |
| N1358K | | | | |
| N1835H | | | | |
| N1938S | | | | |
| N2292K | | | | |
| N2509H | | | | |
| N2979I | | | | |
| N3285D | | | | |
| N3377I | | | | |
| N3626D | | | | |
| N3696S | | | | |
| N3769T | | | | |
| N391K | | | | |
| N3945T | | | | |
| N4536Y | | | | |
| N4915fs | | | | |
| N4929S | | | | |
| N518I | | | | |
| N683D | | | | |
| N880K | | | | |
| N946K | | | | |
| P12Q | | | | |
| P136S | | | | |
| P1421S | | | | |
| P1434S | | | | |
| P15Q | | | | |
| P1643H | | | | |
| P1741S | | | | |
| P1791Q | | | | |
| P1856S | | | | |
| P1941S | | | | |
| P1958H | | | | |
| P2054S | | | | |
| P2064H | | | | |
| P2077R | | | | |
| P2216H | | | | |
| P2269S | | | | |
| P2374H | | | | |
| P2647S | | | | |
| P2648L | | | | |
| P2699S | | | | |
| P2751Q | | | | |
| P2786L | | | | |
| P2832L | | | | |
| P2899S | | | | |
| P3067H | | | | |
| P3099H | | | | |
| P3201S | | | | |
| P3296S | | | | |
| P3553L | | | | |
| P359L | | | | |
| P35L | | | | |
| P3629S | | | | |
| P3776L | | | | |
| P3776S | | | | |
| P3834L | | | | |
| P3868L | | | | |
| P3889L | | | | |
| P3919S | | | | |
| P4117L | | | | |
| P4117Q | | | | |
| P4143S | | | | |
| P4170A | | | | |
| P4331H | | | | |
| P4349L | | | | |
| P4349S | | | | |
| P4377S | | | | |
| P4392S | | | | |
| P4401Q | | | | |
| P4426S | | | | |
| P4434L | | | | |
| P4474A | | | | |
| P447fs | | | | |
| P4501S | | | | |
| P4537H | | | | |
| P4537S | | | | |
| P4543Q | | | | |
| P4559H | | | | |
| P4563L | | | | |
| P4564L | | | | |
| P45S | | | | |
| P4609L | | | | |
| P472L | | | | |
| P473H | | | | |
| P4773L | | | | |
| P4778S | | | | |
| P4784del | | | | |
| P4836T | | | | |
| P636L | | | | |
| P807S | | | | |
| Q1063E | | | | |
| Q1143H | | | | |
| Q1193R | | | | |
| Q1383L | | | | |
| Q1462K | | | | |
| Q1622E | | | | |
| Q1731H | | | | |
| Q1821* | | | | |
| Q2320K | | | | |
| Q235* | | | | |
| Q256P | | | | |
| Q2753* | | | | |
| Q2775H | | | | |
| Q2893R | | | | |
| Q2931H | | | | |
| Q297E | | | | |
| Q3072H | | | | |
| Q3091* | | | | |
| Q3234H | | | | |
| Q3253E | | | | |
| Q3347K | | | | |
| Q3412H | | | | |
| Q3541* | | | | |
| Q4158_splice | | | | |
| Q4221* | | | | |
| Q4475fs | | | | |
| Q453L | | | | |
| Q4739H | | | | |
| Q478K | | | | |
| Q47R | | | | |
| Q4872E | | | | |
| Q557* | | | | |
| R1014* | | | | |
| R1014G | | | | |
| R1060S | | | | |
| R1097I | | | | |
| R1136S | | | | |
| R1163M | | | | |
| R1169Q | | | | |
| R1169W | | | | |
| R122* | | | | |
| R1329I | | | | |
| R1509W | | | | |
| R1579C | | | | |
| R1671C | | | | |
| R1671H | | | | |
| R1679H | | | | |
| R1679L | | | | |
| R1685* | | | | |
| R1685Q | | | | |
| R1698L | | | | |
| R172C | | | | |
| R172H | | | | |
| R1788C | | | | |
| R1788H | | | | |
| R1801Q | | | | |
| R1801W | | | | |
| R1806C | | | | |
| R1806H | | | | |
| R1815C | | | | |
| R1815L | | | | |
| R1826I | | | | |
| R1902* | | | | |
| R1902Q | | | | |
| R1917* | | | | |
| R1917Q | | | | |
| R1929I | | | | |
| R2008W | | | | |
| R2190C | | | | |
| R2190H | | | | |
| R2203Q | | | | |
| R2203W | | | | |
| R2289* | | | | |
| R2289Q | | | | |
| R231W | | | | |
| R2324Q | | | | |
| R2329C | | | | |
| R2329P | | | | |
| R2400M | | | | |
| R2425K | | | | |
| R26* | | | | |
| R2685* | | | | |
| R2685Q | | | | |
| R26Q | | | | |
| R2808I | | | | |
| R2842* | | | | |
| R2844* | | | | |
| R2871K | | | | |
| R2958* | | | | |
| R2958Q | | | | |
| R3004I | | | | |
| R3004S | | | | |
| R308W | | | | |
| R316Q | | | | |
| R3174I | | | | |
| R317C | | | | |
| R317H | | | | |
| R3297H | | | | |
| R3297L | | | | |
| R3325H | | | | |
| R3342* | | | | |
| R3342Q | | | | |
| R3363Q | | | | |
| R336C | | | | |
| R3382I | | | | |
| R3470* | | | | |
| R3470Q | | | | |
| R3522L | | | | |
| R3615L | | | | |
| R3615Q | | | | |
| R3615W | | | | |
| R3716C | | | | |
| R3716L | | | | |
| R3735C | | | | |
| R3735H | | | | |
| R3768Q | | | | |
| R3792W | | | | |
| R3819Q | | | | |
| R3830H | | | | |
| R3830L | | | | |
| R4036* | | | | |
| R4065G | | | | |
| R4121I | | | | |
| R4142K | | | | |
| R4168C | | | | |
| R4168H | | | | |
| R4234Q | | | | |
| R4292K | | | | |
| R430H | | | | |
| R4326G | | | | |
| R4326K | | | | |
| R4460S | | | | |
| R4530fs | | | | |
| R4643C | | | | |
| R4643H | | | | |
| R4653M | | | | |
| R4769H | | | | |
| R4794M | | | | |
| R4799C | | | | |
| R4812M | | | | |
| R4827S | | | | |
| R4866K | | | | |
| R4891* | | | | |
| R4896C | | | | |
| R511C | | | | |
| R555L | | | | |
| R555Q | | | | |
| R555W | | | | |
| R619C | | | | |
| R619H | | | | |
| R633C | | | | |
| R674C | | | | |
| R674H | | | | |
| R856K | | | | |
| S1117L | | | | |
| S1220* | | | | |
| S1262I | | | | |
| S1314C | | | | |
| S1366I | | | | |
| S1366N | | | | |
| S1441L | | | | |
| S1456F | | | | |
| S148C | | | | |
| S1613L | | | | |
| S1655I | | | | |
| S1822F | | | | |
| S1823L | | | | |
| S1842F | | | | |
| S1847F | | | | |
| S1847P | | | | |
| S1950I | | | | |
| S2010G | | | | |
| S2033I | | | | |
| S204F | | | | |
| S2136L | | | | |
| S2313I | | | | |
| S2339* | | | | |
| S2339L | | | | |
| S2389L | | | | |
| S2394L | | | | |
| S2413L | | | | |
| S2506L | | | | |
| S2510F | | | | |
| S2532L | | | | |
| S2537F | | | | |
| S2592Y | | | | |
| S2600P | | | | |
| S2605R | | | | |
| S2683F | | | | |
| S2745L | | | | |
| S2774Y | | | | |
| S2785F | | | | |
| S2785fs | | | | |
| S2810C | | | | |
| S2873N | | | | |
| S2913T | | | | |
| S2965I | | | | |
| S2965N | | | | |
| S3017L | | | | |
| S3017P | | | | |
| S3046F | | | | |
| S3090C | | | | |
| S3092C | | | | |
| S3106F | | | | |
| S3141Y | | | | |
| S3235L | | | | |
| S3414G | | | | |
| S3485L | | | | |
| S3550N | | | | |
| S3556R | | | | |
| S3561G | | | | |
| S3589Y | | | | |
| S3596Y | | | | |
| S3670P | | | | |
| S3691I | | | | |
| S3800Y | | | | |
| S3825F | | | | |
| S3832I | | | | |
| S3885L | | | | |
| S4007G | | | | |
| S4055A | | | | |
| S4090R | | | | |
| S4114Y | | | | |
| S4182L | | | | |
| S424R | | | | |
| S4368F | | | | |
| S4456L | | | | |
| S4483N | | | | |
| S4499F | | | | |
| S4522C | | | | |
| S4650* | | | | |
| S4685R | | | | |
| S4688R | | | | |
| S4690T | | | | |
| S4716N | | | | |
| S4755R | | | | |
| S476* | | | | |
| S4814C | | | | |
| S4815L | | | | |
| S4839C | | | | |
| S4839F | | | | |
| S529T | | | | |
| S621F | | | | |
| S671* | | | | |
| S671L | | | | |
| S706G | | | | |
| S727N | | | | |
| S75R | | | | |
| S931I | | | | |
| S978Y | | | | |
| S979R | | | | |
| T1087S | | | | |
| T1268I | | | | |
| T1312S | | | | |
| T1362S | | | | |
| T1437A | | | | |
| T1516A | | | | |
| T1742M | | | | |
| T1742R | | | | |
| T1866M | | | | |
| T18fs | | | | |
| T1962I | | | | |
| T1993P | | | | |
| T2063K | | | | |
| T2088I | | | | |
| T2169A | | | | |
| T2228P | | | | |
| T2347N | | | | |
| T2409M | | | | |
| T2473I | | | | |
| T2658I | | | | |
| T2792I | | | | |
| T2792S | | | | |
| T2897A | | | | |
| T2897R | | | | |
| T294fs | | | | |
| T3147K | | | | |
| T3163A | | | | |
| T3212S | | | | |
| T3225R | | | | |
| T3267A | | | | |
| T3352N | | | | |
| T3459S | | | | |
| T3472A | | | | |
| T3472I | | | | |
| T3472P | | | | |
| T3499I | | | | |
| T3708I | | | | |
| T3742K | | | | |
| T4049A | | | | |
| T4202I | | | | |
| T4306I | | | | |
| T4306S | | | | |
| T4458K | | | | |
| T4461I | | | | |
| T4514A | | | | |
| T4514I | | | | |
| T4684I | | | | |
| T4694N | | | | |
| T4797K | | | | |
| T4849I | | | | |
| T571P | | | | |
| T643S | | | | |
| T786I | | | | |
| T831S | | | | |
| T882P | | | | |
| V1070I | | | | |
| V134I | | | | |
| V1410M | | | | |
| V1430I | | | | |
| V1546I | | | | |
| V1577A | | | | |
| V1663M | | | | |
| V1707L | | | | |
| V1775I | | | | |
| V1845L | | | | |
| V1845M | | | | |
| V1860L | | | | |
| V2124M | | | | |
| V2140F | | | | |
| V2194A | | | | |
| V2268A | | | | |
| V2282M | | | | |
| V2352I | | | | |
| V2357D | | | | |
| V2398G | | | | |
| V240L | | | | |
| V2459D | | | | |
| V249fs | | | | |
| V2540fs | | | | |
| V2559L | | | | |
| V264I | | | | |
| V2728I | | | | |
| V2740F | | | | |
| V3075L | | | | |
| V3180A | | | | |
| V3187A | | | | |
| V3228L | | | | |
| V3268L | | | | |
| V3369L | | | | |
| V3395M | | | | |
| V3464I | | | | |
| V3699I | | | | |
| V3719I | | | | |
| V3779I | | | | |
| V3798A | | | | |
| V3826E | | | | |
| V3826I | | | | |
| V4243E | | | | |
| V4258I | | | | |
| V4394M | | | | |
| V4509M | | | | |
| V779L | | | | |
| V873M | | | | |
| V879F | | | | |
| V928A | | | | |
| V973I | | | | |
| V986A | | | | |
| W2638C | | | | |
| W29* | | | | |
| W4419* | | | | |
| W4930* | | | | |
| W4936* | | | | |
| W906* | | | | |
| Y1053H | | | | |
| Y1386H | | | | |
| Y1777C | | | | |
| Y1878N | | | | |
| Y2225S | | | | |
| Y2503C | | | | |
| Y2809N | | | | |
| Y3303* | | | | |
| Y3546* | | | | |
| Y3546S | | | | |
| Y3581N | | | | |
| Y3978C | | | | |
| Y4227fs | | | | |
| Y4420H | | | | |
| Y4593C | | | | |
| Y4678C | | | | |
| Y480C | | | | |
| Y4980C | | | | |
| Y588C | | | | |

### Example 3: The Hippo Pathway Mediates Multicellular Resistance to Cytotoxic Drugs

Chemotherapy is widely used for cancer treatment, but its effectiveness is limited by drug resistance. Described herein is a novel role of cell contact-mediated resistance to gemcitabine and several other FDA-approved oncology drugs through the Hippo pathway. Hippo inactivation sensitizes a diverse panel of cell lines and human tumors to gemcitabine in 3D spheroid, mouse xenografts, and patient-derived xenograft models. Nuclear YAP enhances gemcitabine effectiveness by down-regulating multidrug transporters as well by converting gemcitabine to a less active form; both leading to its increased intracellular availability. Cancer cell lines carrying Hippo pathway genetic aberrations showed heightened sensitivity to gemcitabine. Patients, characterized by high expression of genes downstream of YAP evinced prolonged survival. These findings suggest “switching-off” of the Hippo-YAP pathway could present a new opportunity to overcome drug resistance in cancer therapy.

Introduction

Despite the recent excitement surrounding targeted therapy, cytotoxic chemotherapy remains the bedrock of cancer treatment. Ultimately, the efficacy of cytotoxic therapy, like targeted therapy, is limited by drug resistance. Many studies have focused on genetic mechanisms including both intrinsic and acquired means of resistance to chemotherapy. Acquired resistance can occur by genetic mutation during treatment or by selection of preexisting genetic variants in the population. Adaptive responses, such as increased expression of the therapeutic target or activation of compensatory pathways can also influence drug efficacy over time (Holohan et al., 2013). Despite the widespread prevalence of resistance, many oncologists have noted occasional dramatic responses in patients, whom they referred to informally as “exceptional responders” (Chang et al., 2014). Yet, despite the many potential biomarkers and our increasingly sophisticated understanding of the molecular phenotype of the tumor cell, we cannot predict exceptional responders. Instead clinical regimens are still based on prognostic clinico-pathological parameters, such as tumor size, presence of lymph node metastases and histological grade (Weigelt et al., 2012). This state of affairs has produced a growing conviction that the study of drug response and in particular, the exceptional responders, could lead to improvements based on personalizing delivery for targeted and perhaps even for cytotoxic chemotherapies.

The investigation of resistance described herein began with the nucleoside analogue, gemcitabine, which is the first line treatment for locally advanced and metastatic pancreatic cancer (Burris et al., 1997). Regrettably, most pancreatic ductal carcinoma (PDAC) patients treated with gemcitabine do not respond well to treatment. The 1- and 5-year survival rates for pancreatic cancers are about 10% and 4.6%, respectively, which are the lowest survival rates of all major cancers (Burris et al., 1997; Von Hoff et al., 2013). In trying to understand the resistance to gemcitabine and the variable response of patients physiological conditions for pancreatic tumor cells that affected their sensitivity to the drug were unexpectedly found. In each of fifteen pancreatic cancer cell lines that were tested, resistance to gemcitabine very strongly depended on cell crowding. Each cell line was resistant at high density but each was immediately sensitive when re-plated at low density, indicating that the resistance was not due to a preexisting or acquired genetic alteration and led to the characterization of a new physiological means of drug resistance.

Described herein is the profiling of the activity of signaling pathways in six of these lines grown at varying conditions of crowding and the demonstration that increased phosphorylation of YAP was strongly correlated with crowding conditions, consistent with previous observations of the response of Hippo pathway to cell density (Goswami and Nakshatri, 2013). Suppressing the Hippo pathway by expression of a non-phosphorylatable form of YAP or by knockdown of NF2 (an upstream regulator of YAP phosphorylation) sensitized each cell line to gemcitabine, as well as to several other FDA-approved oncology drugs. Furthermore, when the Hippo pathway was inactivated in mouse xenografts of human pancreatic carcinoma cells they became sensitive to gemcitabine. The underlying mechanism by which the Hippo-YAP pathway enhances gemcitabine action included down-regulation of the expression of several multidrug transporters (ABCG2, ABCC3 and MVP) and cytidine deaminase (a key enzyme which metabolizes gemcitabine following its uptake); both lead to increased intracellular concentration of gemcitabine. Overall, these findings highlight a novel role for physiological conditions in mediating sensitivity to gemcitabine; hence, “switching-off” of the upstream regulation of the Hippo-YAP pathway and thus activating YAP could present a new strategy to overcome drug resistance in pancreatic cancer and other cancers.

Results

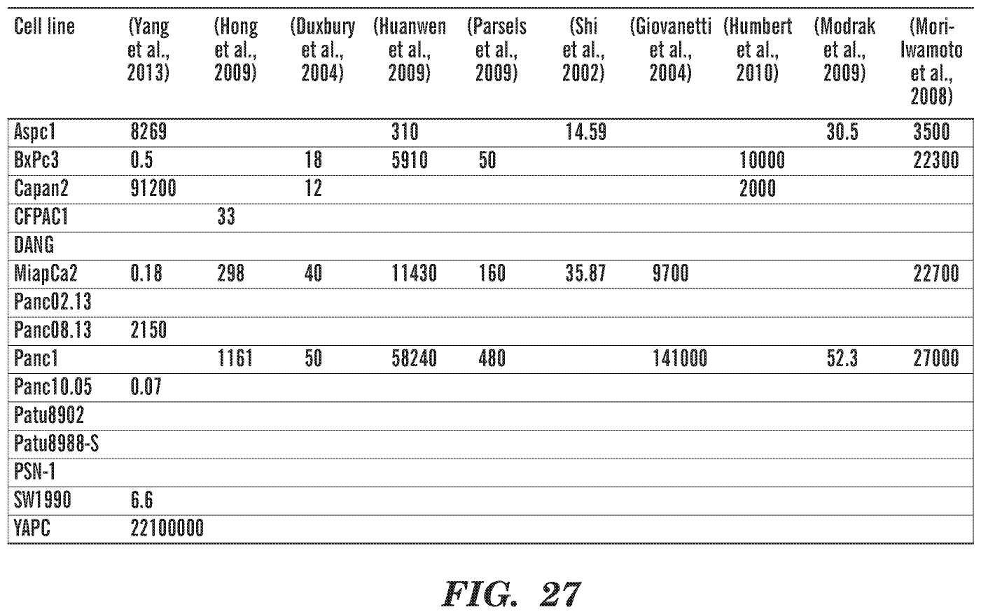

In trying to profile pathways for drug resistance, a large inconsistency in the published studies of the cellular response to gemcitabine was unexpectedly discovered (FIG. 27). The same pancreatic cancer cell line has been reported as sensitive or resistant in different publications; this was true to differing degrees for fifteen cell lines with varying genetic backgrounds. Furthermore, there was little consensus among published large scale Cancer Genome Project (CGP) studies that measured affect of gemcitabine on a large panel of genomically annotated cancer cell lines (Garnett et al., 2012; Haibe-Kains et al., 2013). Since varying assay conditions such as end time point, detection method and seeding density were used in these previous studies, these studies were repeated herein using a real-time (kinetic) cell growth assay.

Cell-Cell Contact-Dependent Response to Gemcitabine in Pancreatic Cancer

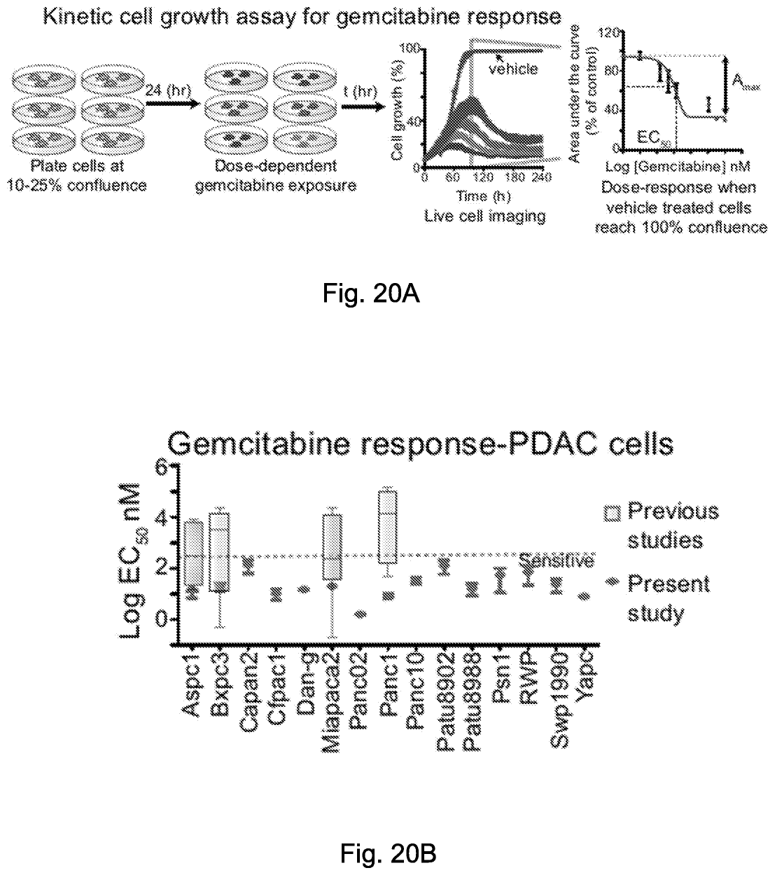

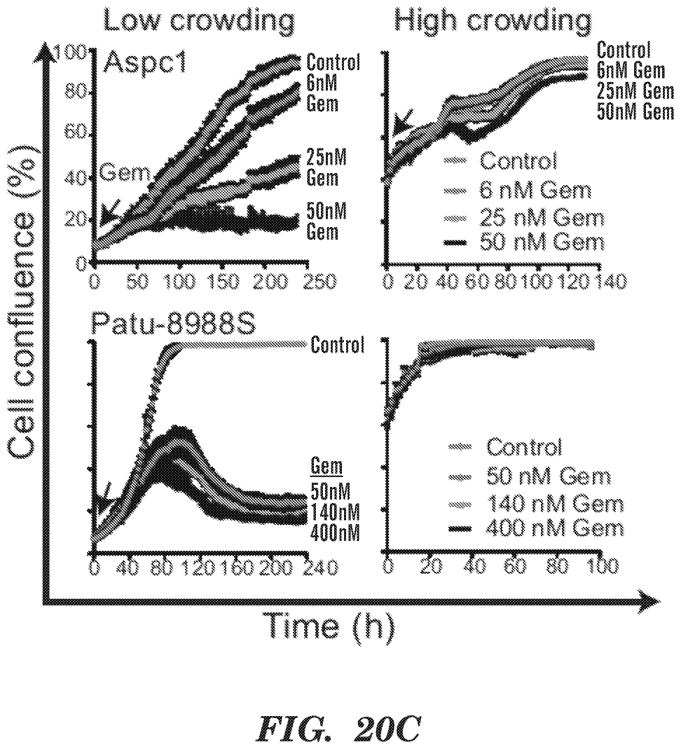

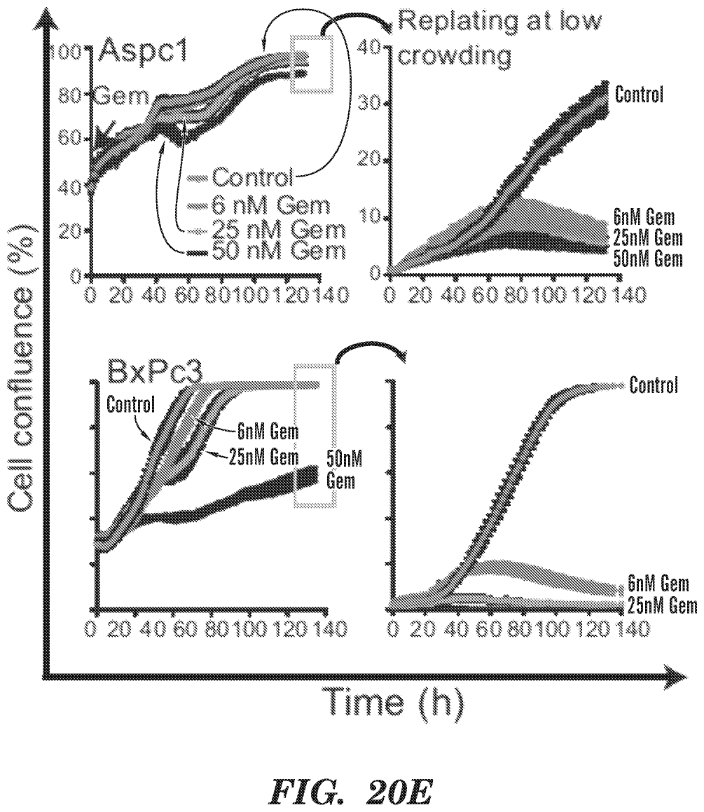

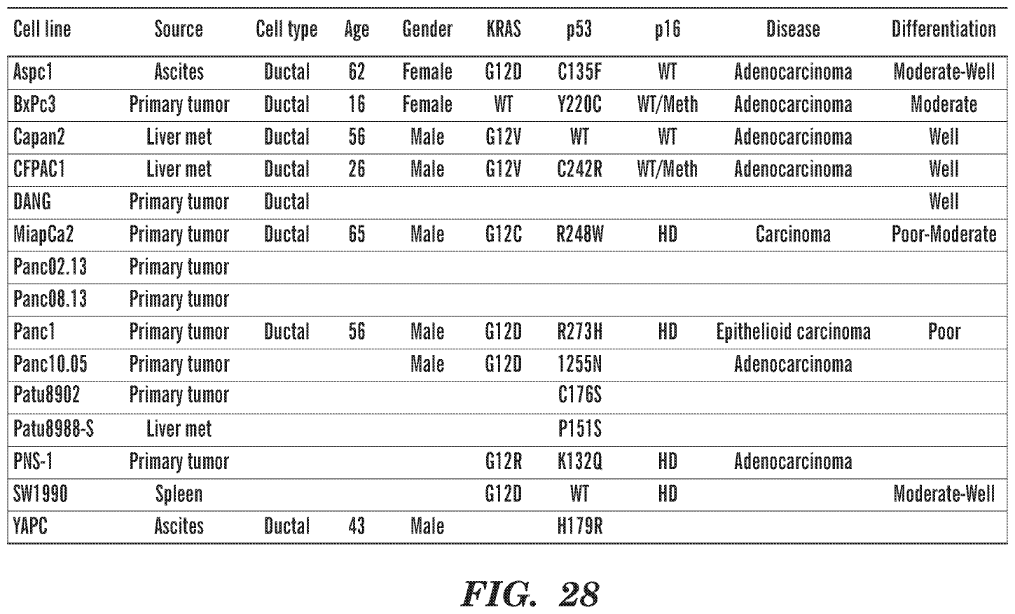

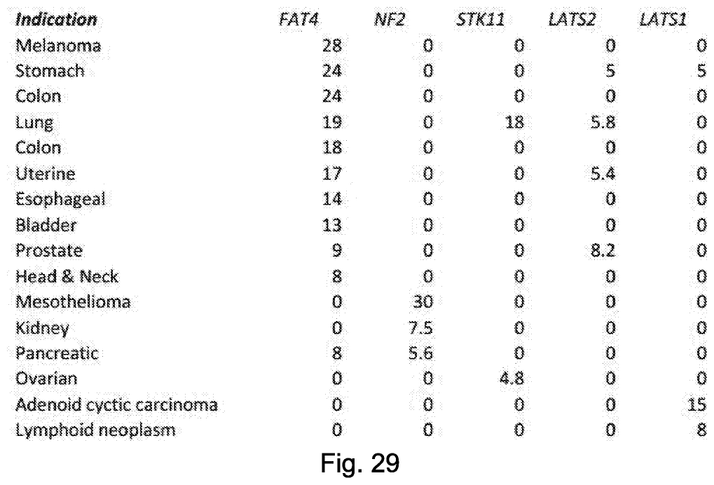

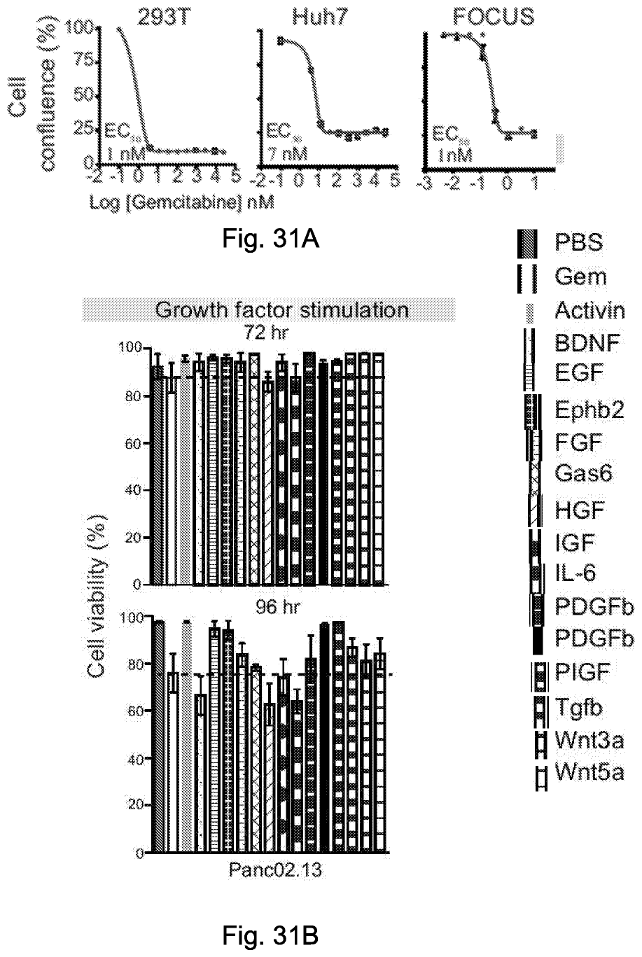

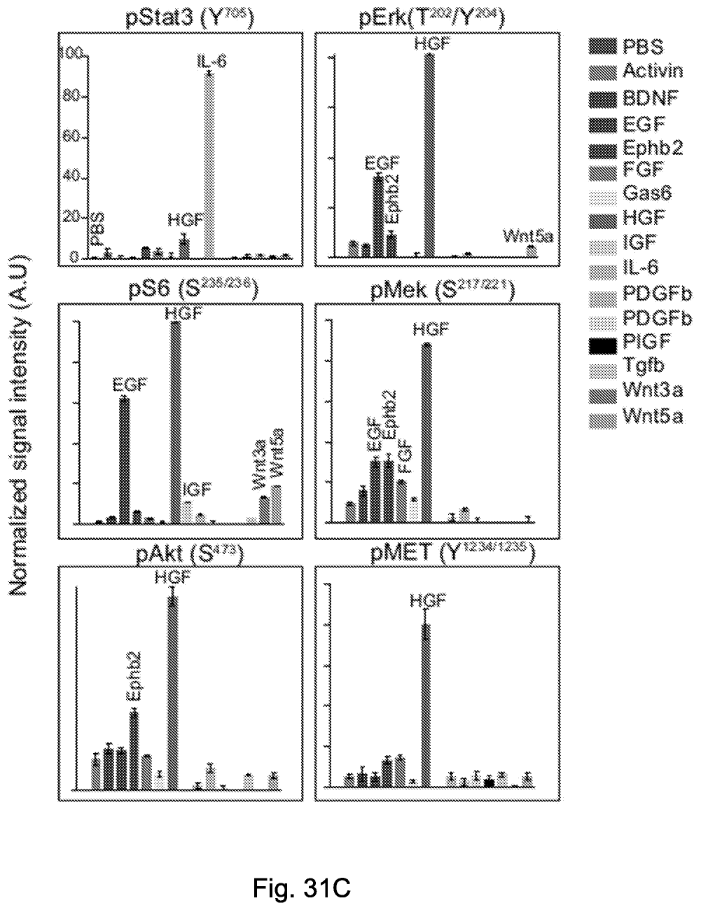

FIG. 20A illustrates the kinetic cell growth assay to determine the effect of gemcitabine in a panel of pancreatic cancer cell lines. Cells are plated at low crowding conditions (10-25% confluence) and 24 hours later exposed to gemcitabine in a dose-dependent manner. They are imaged every 1-3 hours until control (vehicle) treated cells reach 100% confluence. This assay is not confounded by the fact that the time required for each cell line to reach 100% confluence may be very different (as the cell lines have different doubling times). A dose response effect of gemcitabine on cell growth for 16 pancreatic cancer cell lines is shown in FIGS. 20B, 3, 31A-31C and 28, where the range of previous studies is also shown. In our experiments all cell lines tested under these conditions were sensitive to gemcitabine (EC50<200 nM) (FIG. 20B, 3, 31A-31C). Similar responses to gemcitabine were found in liver cancer cell lines (Huh7 and FOCUS) and untransformed (HEK293) cell lines (FIGS. 3, 31A-31C).

In the course of these experiments it was inadvertently found that cells grown in more crowded conditions (40-60% confluence) were much less sensitive to gemcitabine, relative to cells grown in less crowded conditions (10-25% confluence) (FIG. 20C). Every PDAC cell line showed this effect. This was reflected in the EC50 as well as the Amax, as shown in FIG. 20D, which demonstrates the striking disparity of sensitivities at high and low density.

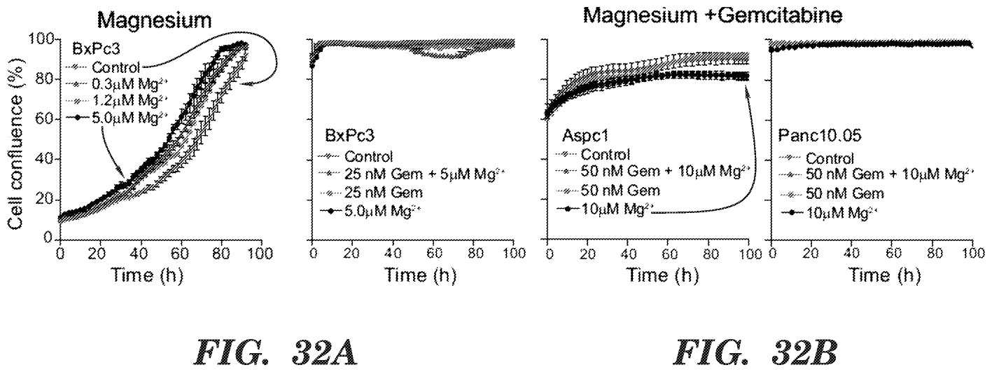

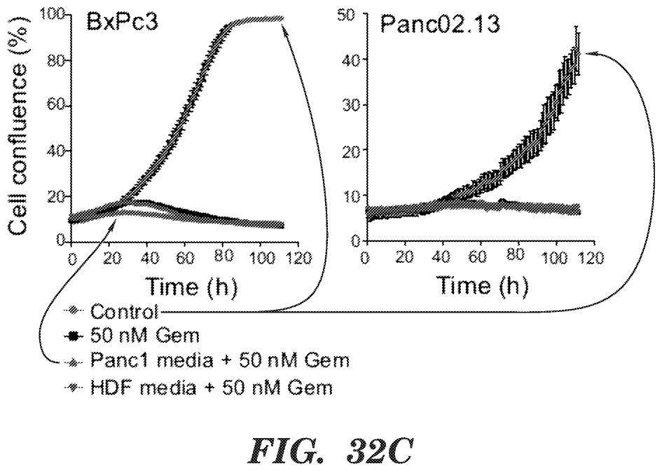

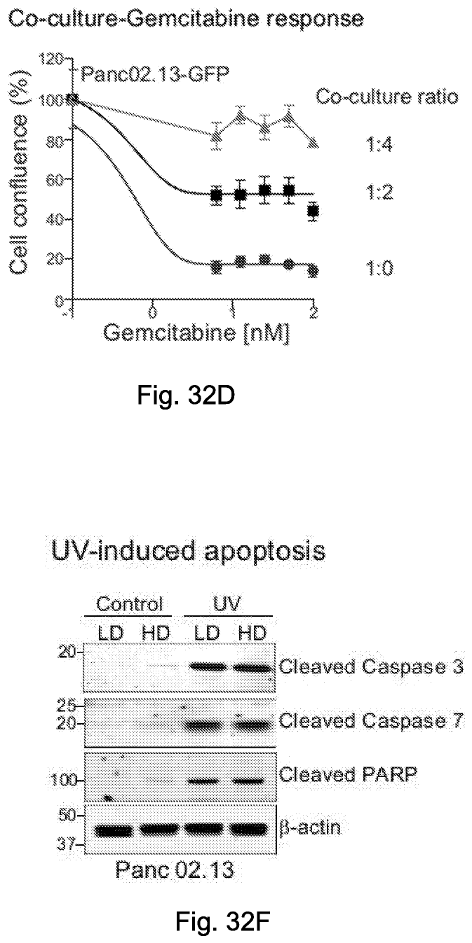

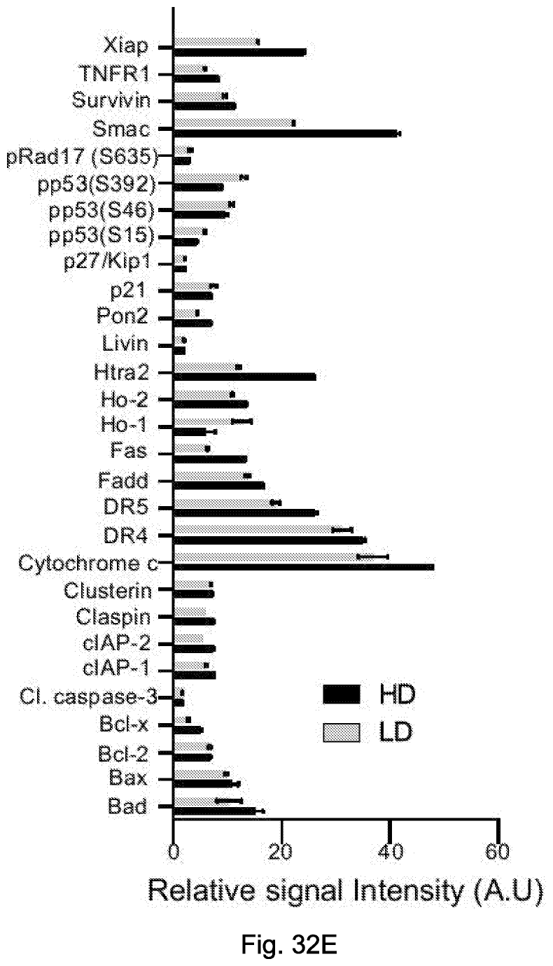

The in vitro crowding conditions had no obvious relevance to the growth conditions in human tumors. Nevertheless, it was investigated how extrinsic factors could so dramatically affect drug sensitivity. One possible explanation was depletion of the culture medium. A change of medium or addition of insulin or fresh serum has been shown to produce a balanced stimulation of macromolecular synthesis and cell division in post-confluent cultures (Griffiths, 1972; Leontieva et al., 2014; Sanford et al., 1967). Replenishing fresh medium, containing serum or supplemented with 15 different growth factors, including EGF, FGF, IGF, HGF, PDGF, Wnt3a, Wnt5a, TGFβ, and IL 6 did not increase the sensitivity of insensitive cells at high-density conditions to gemcitabine (FIGS. 31A-31C). Yet these growth factors had activated their cognate downstream signaling proteins even in the high crowding conditions (FIGS. 31A-31C). For example, stimulation of IL 6 led to phosphorylation of Stat3 while stimulation with HGF and EGF caused increased phosphorylation of ERK, MEK and S6 proteins (FIGS. 31A-31C). In addition, Mg++ concentration, which had also been shown to play a role in modulating protein and DNA synthesis and cell proliferation in cultured cells (Rubin, 2005), also did not increase susceptibility to gemcitabine. Though supplemental Mg++ can cause a marginal increase in the growth, it had no affect on gemcitabine sensitivity in Bxpc3, Aspc1 and Panc10.05 cells (FIGS. 32A-32F). Conditioned media from dermal fibroblasts has recently been shown to cause gemcitabine resistance in colorectal and pancreatic cancer cells, implying that changes in the tumor microenvironment could alter drug resistance (Straussman et al., 2012). Yet exposure of pancreatic cancer cells to the conditioned media of human dermal fibroblast, vascular endothelial cells, or other mesenchymal cancer cells (Pancl) had no affect on gemcitabine response in Bxpc3 and Panc02.13 cells (FIGS. 32A-32F). Finally, co-culturing of sparse GFP-labeled Pan02.13 cells with fibroblast or other cancer cells to achieve high overall cell density produced the same resistance to gemcitabine found in dense tumor cell culture (FIGS. 32A-32F). These data indicate that a wide variety of extrinsic cell growth conditions do not affect the sensitivity of pancreatic cancer cells to gemcitabine in crowded conditions.

It was considered that pancreatic cancer cells might have become temporarily resistant to apoptosis in high-density growth conditions. There is no change in the protein levels of 29 apoptotic signaling proteins including Bad, Bax and Bcl2 in response to crowding conditions (FIGS. 32A-32F). Furthermore, Panc02.13 cells exposed to UV radiation in crowded conditions underwent apoptosis as assessed by cleaved caspase 3, 7 and PARP levels (FIGS. 32A-32F), indicating that crowded cells are not intrinsically resistant to apoptosis. Finally, re-plating Aspc1 and Bxpc3 cells at low density (using the original growth medium containing gemcitabine) immediately re-established their sensitivity (FIG. 20E), further indicating that the gemcitabine response in pancreatic cancer cells is a function of cell crowding and not dependent on extrinsic cell culture conditions.

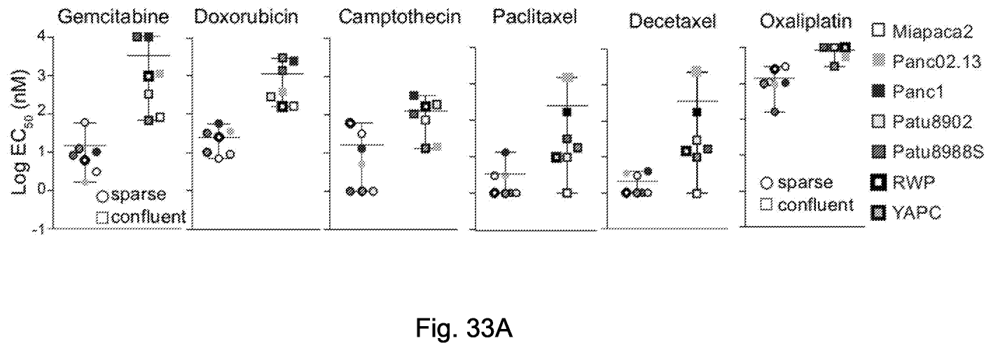

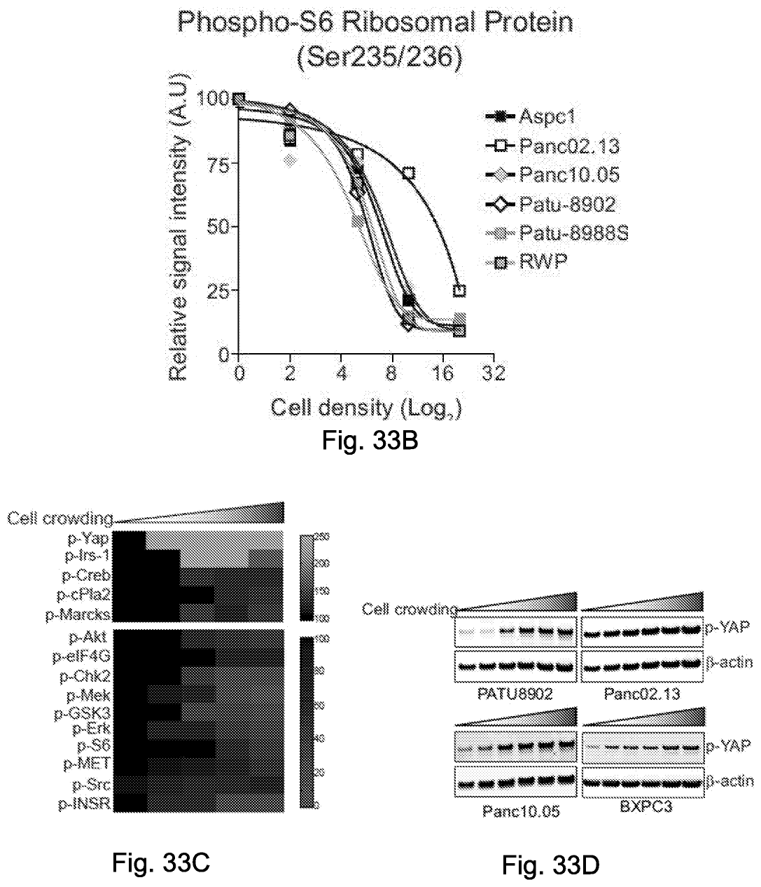

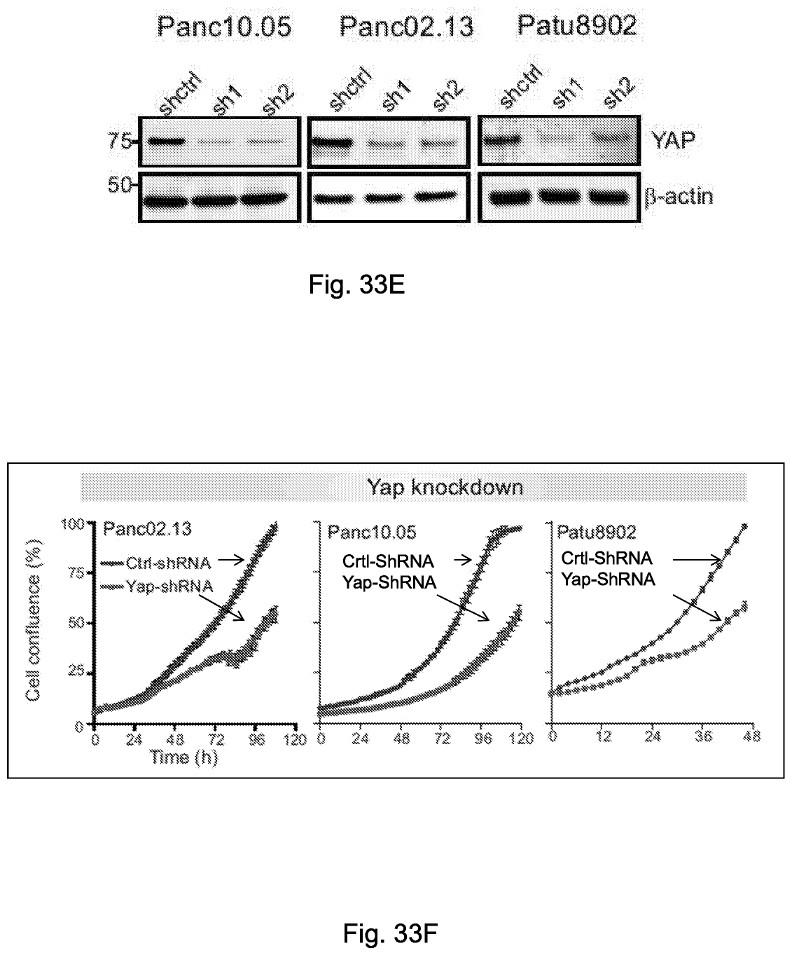

To establish whether the effect of crowding is related to some very special characteristic of gemcitabine's mechanism of action, the effect of cell crowding on a set of 7 diverse cytotoxic drugs was examined. The sensitivity of seven PDAC cell lines grown at varying crowding conditions to these 7 cytotoxic drugs, commonly used in chemotherapy, was tested. The cellular response to both gemcitabine and doxorubicin (a topoisomerase II inhibitor) was dependent on cell crowding (using a >100 fold difference in EC50 as the threshold) while the response to camptothecin, paclitaxel, docetaxel (taxane) and oxaliplatin (platinum) showed weak or no correlation with cell density (FIGS. 33A-33E). That several cytotoxic inhibitors such as taxanes were equally sensitive in low or high crowding conditions further corroborates the conclusion that cells in high crowding conditions are susceptible to apoptosis (FIGS. 33A-33E). Overall, these data indicate that the cellular response of pancreatic cancer cells to cytotoxic drugs, such as gemcitabine is greatly influenced by cell-cell interactions and that this property is shared by some but certainly not by all cytotoxic drugs.

The Hippo-YAP Pathway Controls Sensitivity to Gemcitabine.

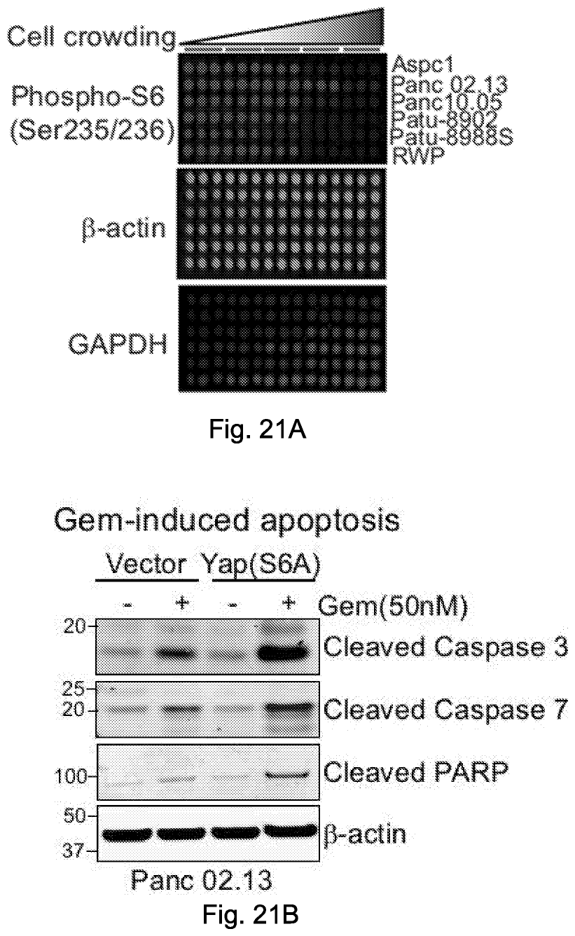

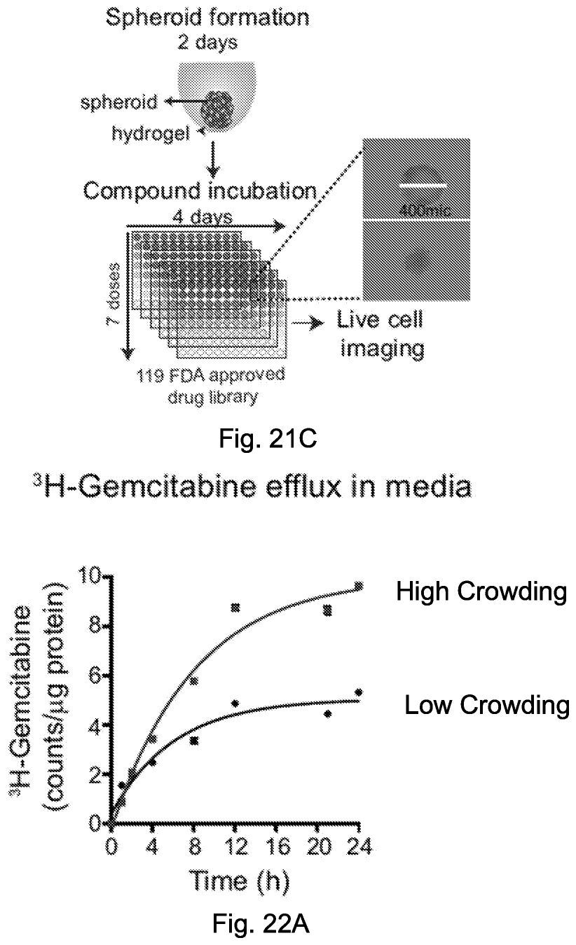

To identify signaling pathways that might mediate the density dependent responses to gemcitabine, reverse-phase protein arrays were used to measure the activity of 75 signaling proteins in a panel of six pancreatic cancer cell lines grown in various crowding conditions (FIG. 21A). As expected, when cell growth is slowed down at high cell density the activities of many growth factor signaling proteins such as Erk, Akt and S6 ribosomal proteins are down-regulated (FIGS. 21A, 5, and 33A-33F). More interestingly, an increase (>10-fold) in phosphorylation of Yes-associated protein (YAP) was observed at increased cell density (FIG. 5), which was confirmed by Western blotting in several PDAC cell lines (FIGS. 33A-33F). Smaller but highly significant increases in the levels of glycolytic enzymes were also observed. YAP is a potent transcriptional co-activator that functions via binding to the TEAD transcription factor in the Hippo pathway; it plays a critical role in the control of organ size and in tumorigenesis (Camargo et al., 2007; Zhao et al., 2010). Pathway activation inactivates the YAP protein. In this circumstance phosphorylation of YAP by upstream kinases, such as the LATS kinases, causes YAP to be excluded from the nucleus and be retained or degraded in the cytoplasm, where it can no longer activate transcription (Hao et al., 2008). YAP localization was already known to be controlled by cell density (Zhao et al., 2007). In agreement with these observations we observed crowding-dependent nuclear localization of YAP in pancreatic cancer cells—that is, nuclear localization was only found in cells at low confluence (data not shown).

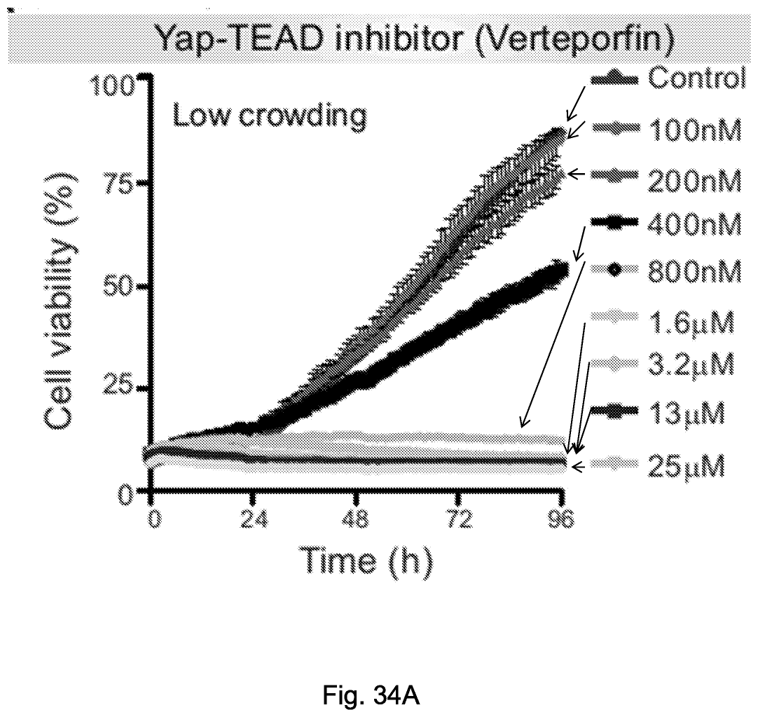

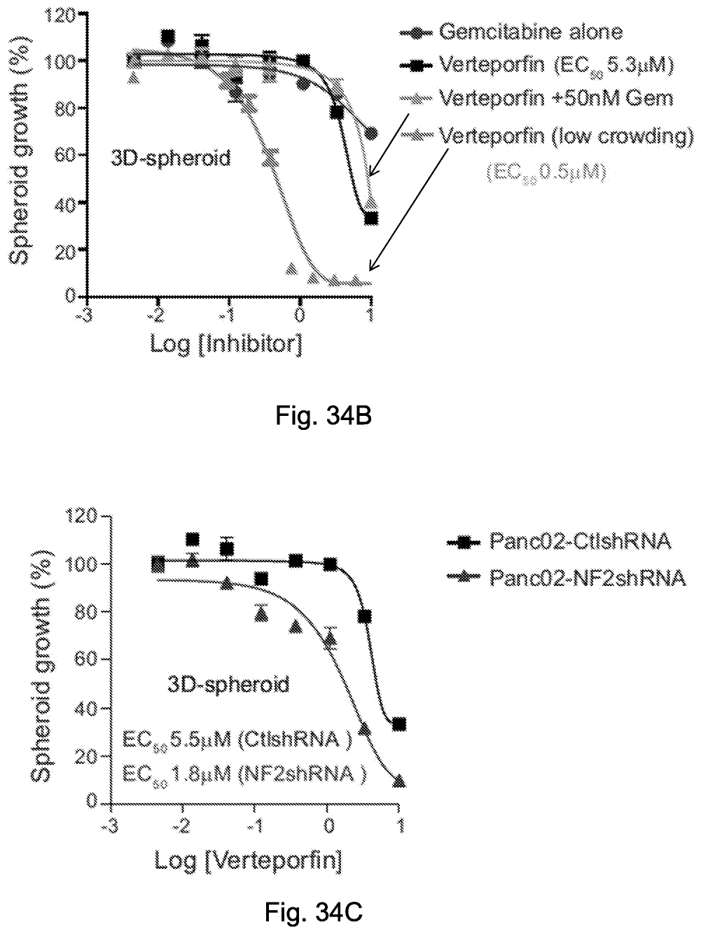

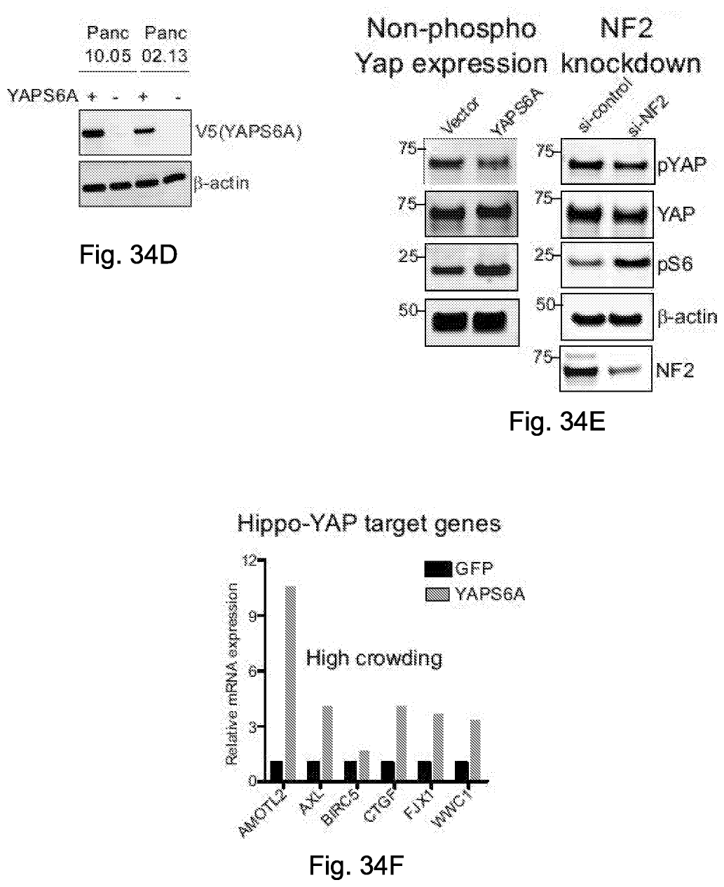

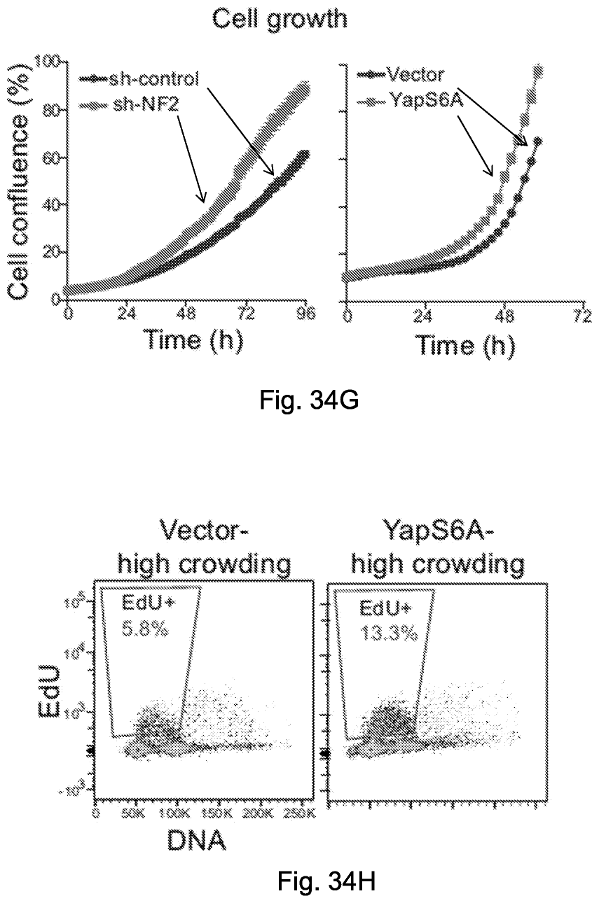

Although there is increasing evidence for a role of the Hippo pathway in cell proliferation, the observed effects here, particularly at high density where the cells are resistant to gemcitabine, is a previously uncharacterized feature of this pathway. Although knockdown of YAP in three different pancreatic cancer cell lines mildly depressed proliferation (FIGS. 33A-33F), it had no effect on gemcitabine response. It was also known that Hippo pathway inactivation (YAP in the nucleus) can trigger tumorigenesis in mice and that altered expression of a subset of Hippo pathway genes can be found in several human cancers (Harvey et al., 2013). When the Hippo pathway is inactivated YAP is localized in the nucleus in 60% of hepatocellular carcinomas, 15% of ovarian cancers and 65% of non-small-cell lung cancers (Harvey et al., 2013). However, only a small fraction of human pancreatic tumors exhibited intense nuclear staining for YAP in late-stage tumors (Zhang et al., 2014). Without wishing to be bound by theory, it is contemplated that the human tumors show the “crowded, gemcitabine-resistant phenotype.” Consistent with the nuclear localization when cells were grown at low density, verteporfin (a YAP-TEAD small molecule inhibitor) (Liu-Chittenden et al., 2012) treatment had a potent affect on pancreatic cancer cell growth in low density growth conditions (Hippo-Off, EC50, <0.5 μM), but had little effect on pancreatic cancer cell growth in 3D-spheroid assays (Hippo-on, EC50, >5 μM) (FIGS. 34A-34H).

In cells at low cell density, where YAP is localized to the nucleus, presumably YAP dependent transcription is turned on. At high density, YAP is in the cytoplasm, transcription is blocked and resistance to gemcitabine is high. Given these correlations it was asked whether inactivation of Hippo pathway could restore gemcitabine sensitivity in crowded conditions. Expression of a non-phosphorylatable form of YAP (YAPS6A) in Panc02.13 pancreatic cancer cells causes constitutive nuclear localization of exogenous YAP even at high crowding (data not shown). Expression of YAPS6A in crowded cells led to increase in expression of YAP-TEAD target genes including AMOTL2 (>10-fold), CTGF (>3-fold), AXL (>3-fold), and BIRC5 (>2-fold (FIGS. 34A-34H). While cells expressing the YAPS6A mutant or knockdown of NF2 (an upstream stimulator of YAP phosphorylation)(Zhang et al., 2010) showed altered morphology and a mildly increased rate of cell growth (FIGS. 34A-34H), the increased sensitivity to gemcitabine (and 5-flurouracil) as measured by growth retardation or increased apoptosis was much more striking (FIG. 6, 21B, 35A-35-35H). NF2 depletion in Panc02.13 cells also restored sensitivity to verteporfin in a high-density spheroid assay (FIGS. 34A-34H). Together, these data indicate YAP phosphorylation (and its export from the nucleus) is the critical determinant of resistance to gemcitabine and perhaps other drugs.

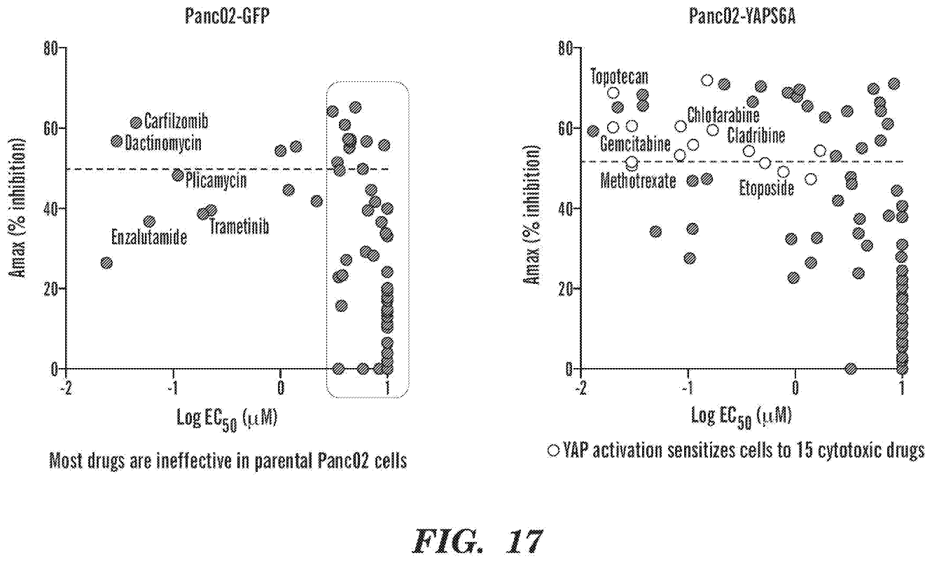

To determine if the Hippo-YAP pathway regulates the sensitivity of pancreatic cancer cells to a broader set of oncology drugs, 119 FDA-approved oncology drugs were screened using the 3D-spheroid (high crowding condition) assay. In this assay, cells were plated in a round-bottom, hydrogel coated wells for 2 days to form compact 3D spheroids (FIG. 21C). Cells were then treated with small molecule inhibitors at varying concentrations (10⁻⁹-10⁻⁵M) and imaged over 4 days (FIG. 21C). A dose response curve for each inhibitor is calculated based on control (no inhibitor/DMSO) treated wells. Most of the inhibitors tested were ineffective in blocking the growth of Panc02.13 cells (EC50, >1000 nM; Amax, <50%) Only carfizomob and dactinomycin showed significant inhibition in these high density growth conditions (FIG. 17). To test the role of the Hippo pathway in regulating sensitivity Panc02.13 cells expressing the YAPS6A mutant were then exposed to the same drugs. 15 drugs showed significantly enhanced sensitivity (EC50, <1000 nM; Amax, >50%) (FIG. 17, 35A-35H). These inhibitors include antimetabolites, anthracyclines, topoisomerase inhibitors and kinase inhibitors, indicating that the role of the Hippo pathway in altering the efficacy is not simply related to the drug's mechanism of action.

The Hippo-YAP Pathway Modulates Gemcitabine Metabolism and Export.

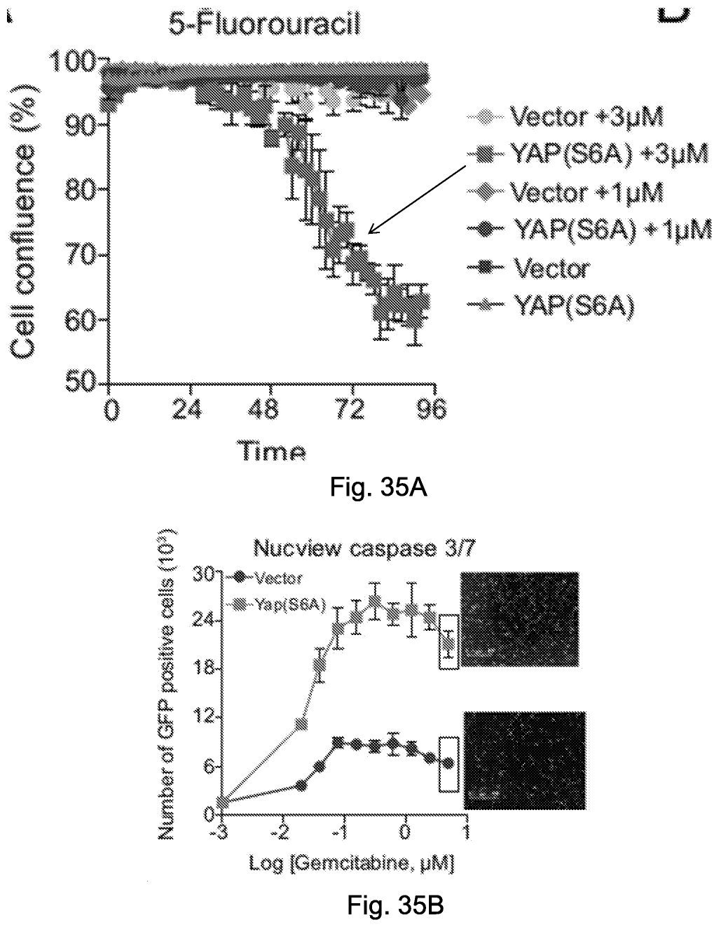

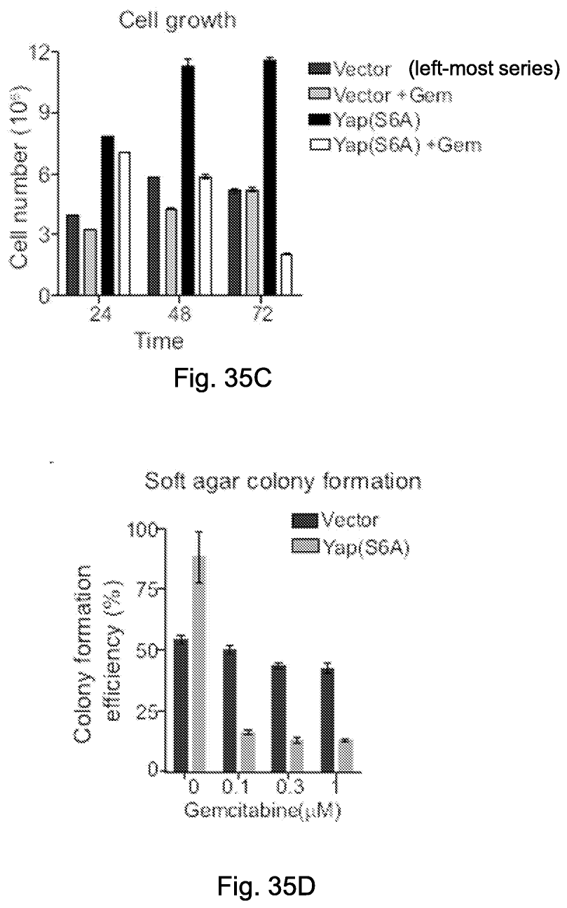

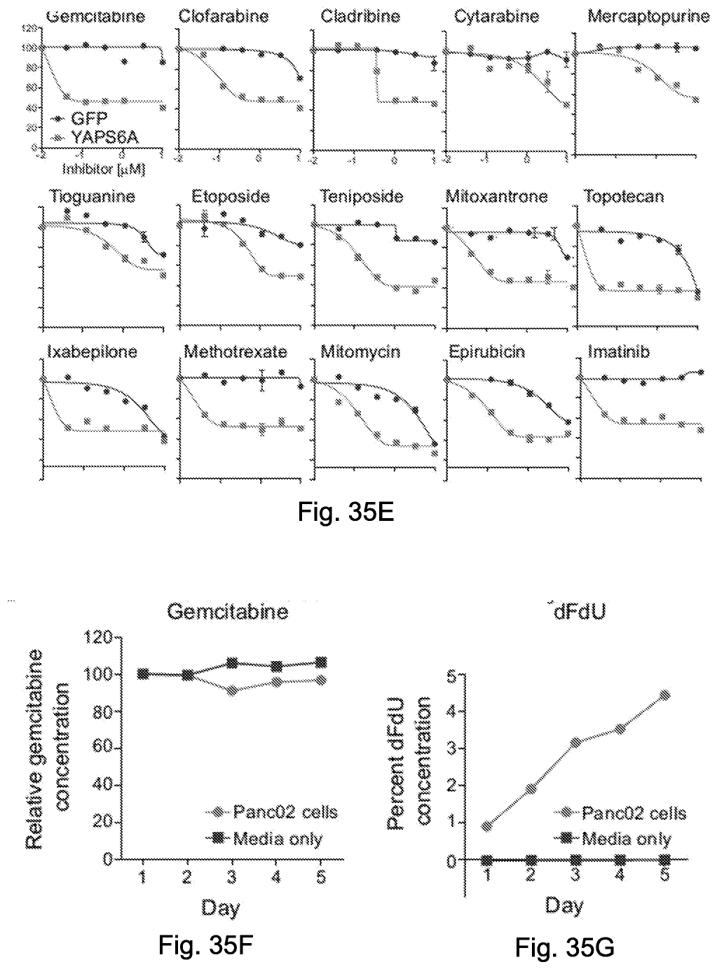

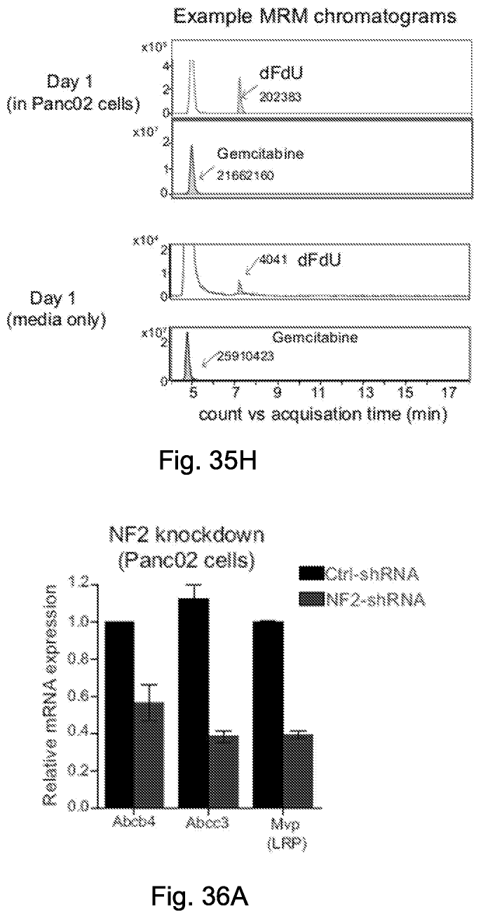

The diverse chemotypes affected by the Hippo pathway, suggested more of a general process of drug availability rather than regulation of a specific cellular pathway. Drug availability mediated by transport or binding or export from the cell is known to be a major determinant of the sensitivity to chemotherapy (O′CONNOR, 2007). It was checked that gemcitabine was not lost from the medium due to lability or enzymatic degradation and found that gemcitabine is not labile in culture media (FIGS. 35A-35H). Furthermore, conditioned media collected from Panc02.13 cells exposed to gemcitabine after 5 days retained 96.7% activity (FIGS. 35A-35H).

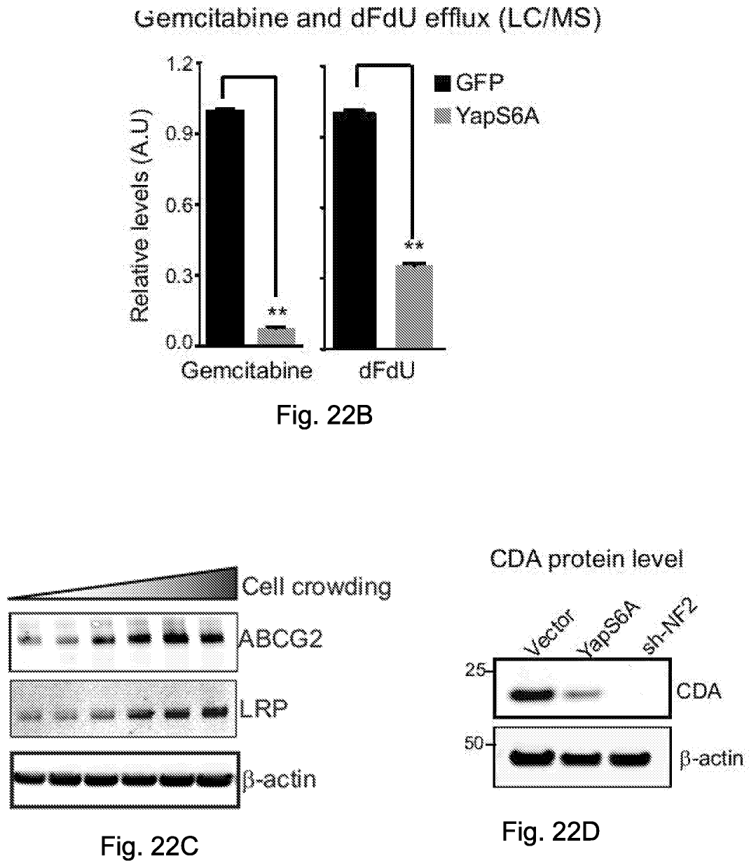

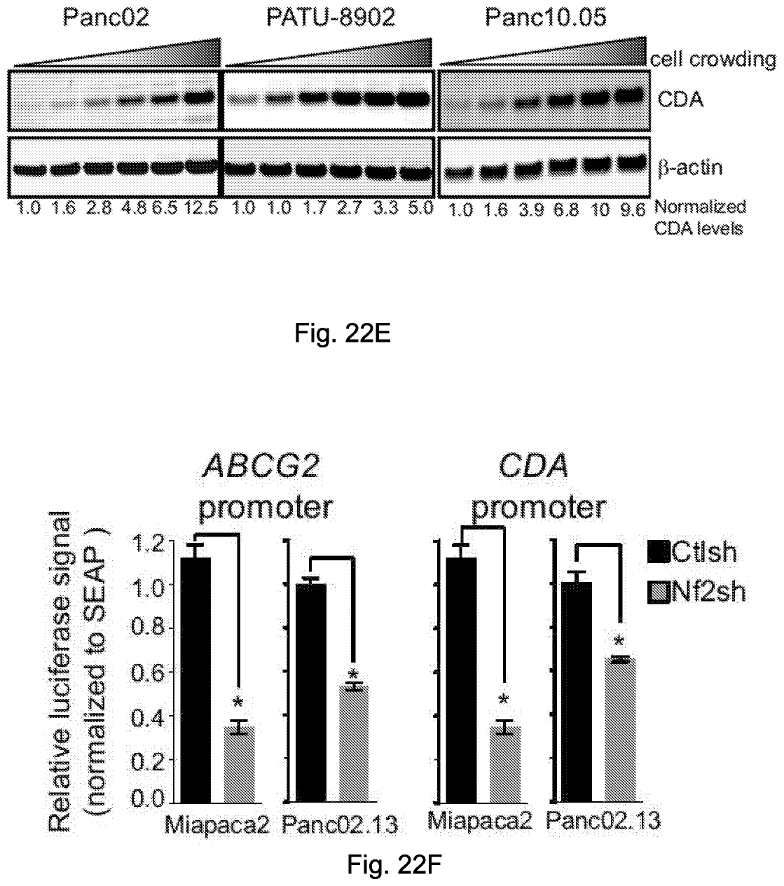

It was considered whether the Hippo pathway might affect the efflux of gemcitabine and/or its metabolites. To assess directly gemcitabine efflux in conditioned media of pancreatic cancer cells both radiolabeled gemcitabine and LC-MS/MS-based methods were used. Panc02.13 cells grown in highly crowded conditions (Hippo-ON) pumped out 2-3-fold more radiolabeled gemcitabine (counts per μg protein) compared with cells grown in less crowded conditions (Hippo-OFF) (FIG. 22A). Another pathway of inactivation and export is the enzymatic conversion of gemcitabine to a uracil derivative (2′,2′-difluorodeoxyuridine, dFdU) by deamination catalyzed by cytidine deaminase (CDA)(Veltkamp et al., 2008). The efflux of gemcitabine and its deaminated metabolite, dFdU was measured by LC-MS/MS (24) in Panc02.13 cells expressing YAPS6A or vector control after gemcitabine treatment (FIG. 22B). Panc02.13 cells expressing YAPS6A (Hippo-OFF) effluxed significantly less gemcitabine (10-fold, p<0.05) compared with GFP expressing cells, in agreement with the radiolabel measurements (FIG. 22B). YAPS6A expressing Panc02.13 cells also effluxed significantly less dFdU (5-fold, p<0.05) compared with GFP expressing cells. Together, these data indicate that activation of the Hippo-YAP pathway in high-density cultures increases efflux of gemcitabine and its metabolic conversion to dFdU resulting in a lower intracellular gemcitabine concentration (FIG. 22B).

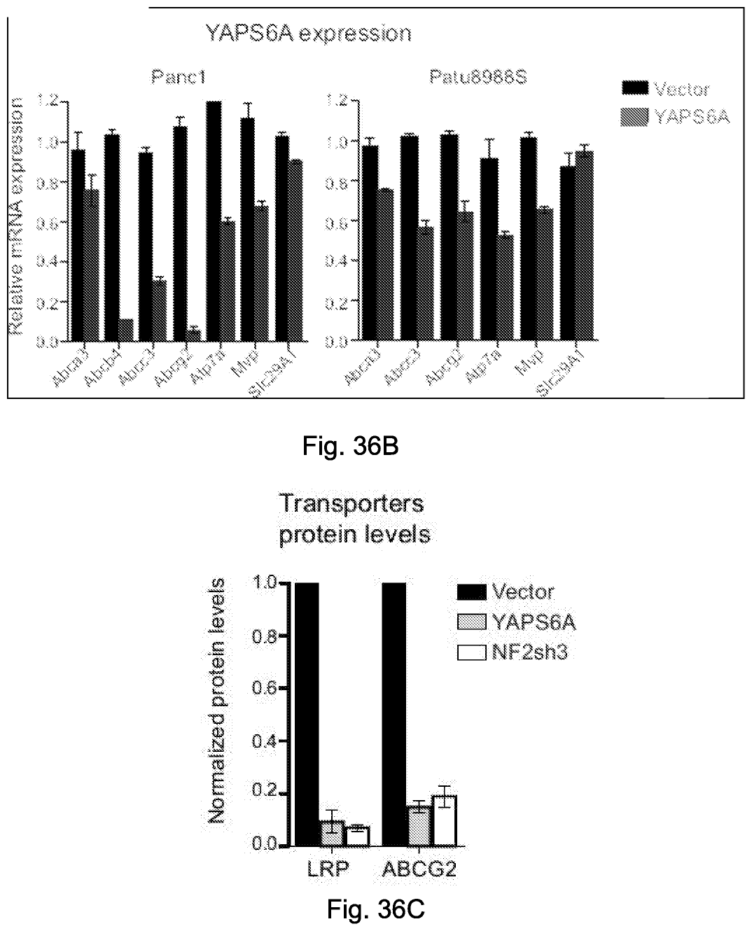

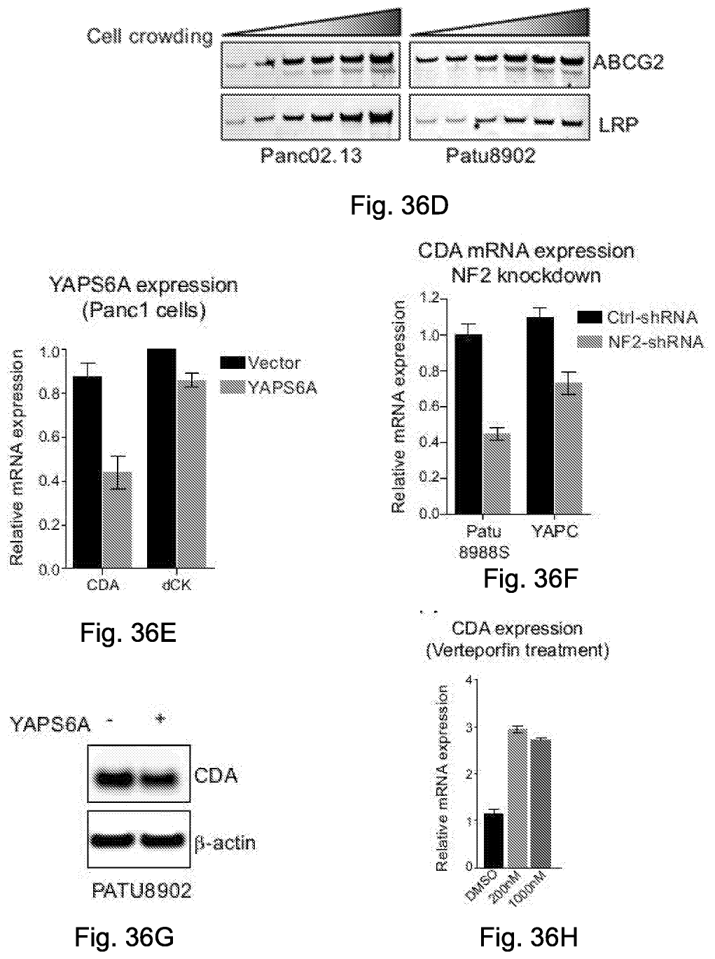

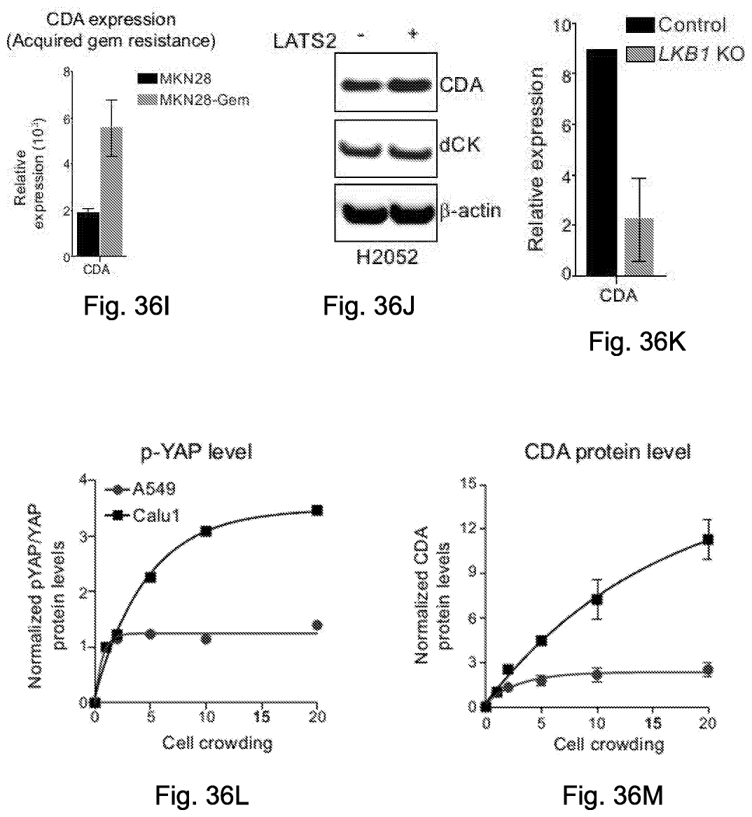

Drug efflux transporters can reduce the concentration of cytotoxic drugs in the cell, allowing cancer cells to survive (Polli et al., 2008). It was investigated by quantitative PCR which transporters might be regulated by the Hippo pathway by profiling the expression of 84 drug efflux transporters in Panc02.13 cells expressing YAPS6A or a control vector. Those include the ABC (ATP-binding cassette) transporters, SLC (solute-carrier) transporters and other transporters, such as voltage-dependent anion channels, aquaporins, and copper pumps. It was found that the mRNA expression levels of eight transporters, mostly from the ABC transporter family, significantly decreased (4-16-fold, p<0.05) in Panc02.13 cells expressing the YAPS6A mutant vector compared with GFP expressing cells (FIG. 9). Quantitative Western blotting also confirmed these findings and revealed that the protein levels of these receptors were reduced when the Hippo pathway is inhibited (FIG. 36A-36M). Similar results were seen in Pancl, Patu8988S, and Patu8902 cells (FIGS. 36A-36M). Many of these transporters including ABCG2, ABCC3 and LRP (lung cancer resistance protein), have previously been implicated in gemcitabine resistance and/or are highly expressed in pancreatic tumors (Hagmann et al., 2010; Rudin et al., 2011; Zhao et al., 2013). Expression levels of the monocarboxylate transporter (SLC3A2), the antigen peptide transporter (TAP2), and an amino acid transporter (SLC16a1) were mildly increased (2-4-fold, p<0.05) in Panc02.13 expressing the YAPS6A construct (FIG. 9). Since cell crowding inhibits the phosphorylation and activity of YAP, which then is retained in the nucleus (FIG. 5) (Zhao et al., 2007), it would be expected that the expression of these drug transporters (ABCG2, LRP and ABCC3) would be significantly increased (FIGS. 22C, 36A-36M). On the other hand the mRNA levels of uptake transporters for gemcitabine (SLC29A1, SLC29A2) were not affected by cell crowding or YAP activity (FIGS. 36A-36M). These data indicate activation of Hippo pathway during crowding decreases the expression of drug efflux transporters, thereby increasing the effective intracellular concentration of gemcitabine.

The activity of the Hippo pathway not only affected the efflux of gemcitabine but also its major metabolite, dFdU (FIG. 22B). Switching-off the Hippo pathway (by depletion of NF2 or expression of YAPS6A) significantly decreased both the mRNA (5-8-fold, p<0.05) and protein levels (5-10-fold, p<0.05) of cytidine deaminase; these changes also increase gemcitabine levels (FIGS. 12, 22D). Similar results were seen in four other pancreatic cancer cell lines (Pancl, Patu8988S, YAPC, and Patu8902 (FIGS. 36A-36M). By contrast, the level of deoxycytidine kinase (dCK, the enzyme involved in the first phosphorylation and activation of gemcitabine) was not affected by the Hippo pathway (FIGS. 12, 36A-36M). Consistently, cell crowding increased the levels of CDA (5-10-fold, p<0.05) in several other pancreatic cancer cell lines (FIG. 22E), which should contribute to the drop in gemcitabine levels and drug resistance. Finally, verteporfin treatment of Panc02.13 cells, which should phenocopy high density by inactivating YAP, led, as expected, to a significant increase in CDA levels (3-fold, p<0.05) (FIGS. 36A-36M), indicating that expression of CDA is negatively regulated by the Hippo pathway and probably not a direct result of treatment with a nucleoside analog.

To further delineate the molecular mechanism of how the Hippo pathway might regulate the levels of gemcitabine efflux pumps and the deaminase enzyme, TEAD binding sites were assessed in the promoter region of ABCG2 and CDA. Transcription factor ChIP-seq data from the Encyclopedia of DNA Elements (ENCODE) (2012) revealed multiple TEAD4 consensus binding sites in the promoter region of ABCG2, ABCC3, LRP and CDA. To validate these findings synthetic promoter activity constructs comprising of promoter region of either ABCG2 or CDA followed by luciferase gene were designed. Promoter activity of both ABCG2 and CDA was significantly decreased in cells expressing YAPS6A mutant in both Panc02.13 (2-fold, p<0.05) and Miapaca2 (3-fold, p<0.05) cells compared with GFP vector expressing cells (FIG. 22F). These data indicate that Hippo-YAP pathway affects gemcitabine action by negatively regulating mRNA expression of drug resistance proteins as well as CDA, thereby modulating export and metabolism of gemcitabine.

Indications that Inhibition of Hippo-YAP Pathway Activity Increase Sensitivity to Gemcitabine in Human Tumors

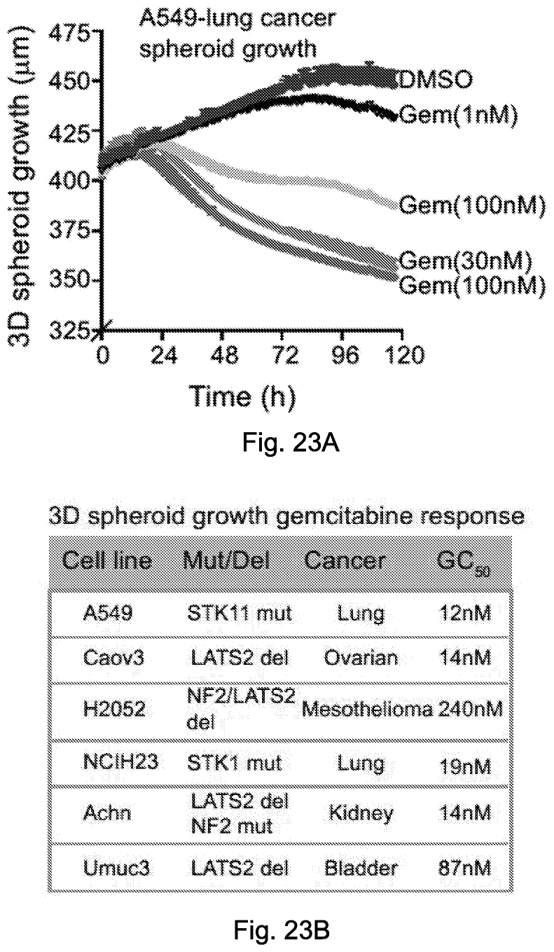

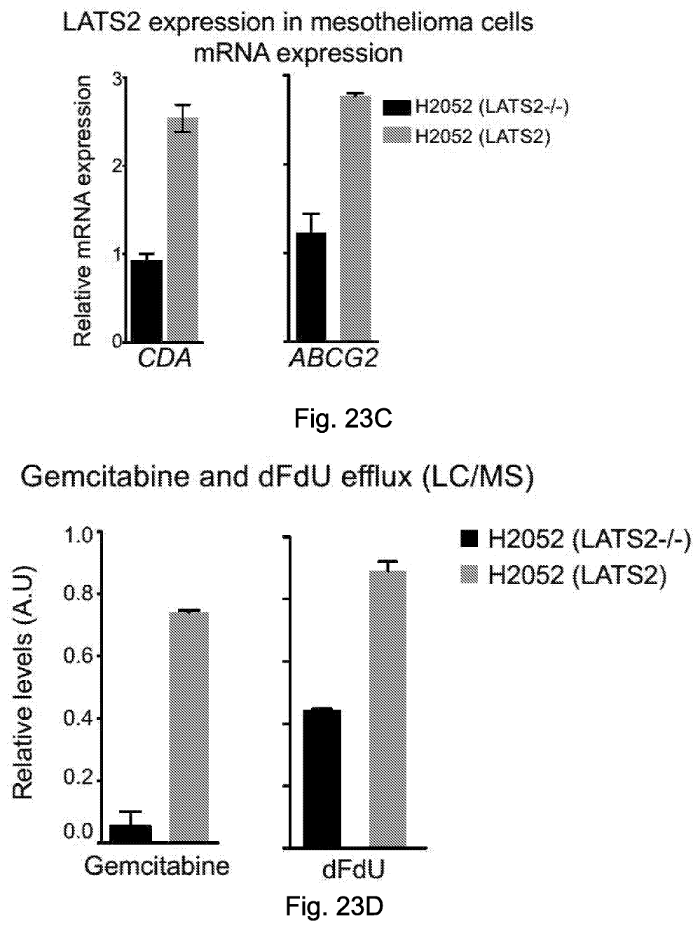

Genetic defects that inhibit the Hippo pathway can induce tumors in model organisms. Such mutations occur in a broad range of human carcinomas, including lung, mesothelioma, colorectal, ovarian and liver cancers (Harvey et al., 2013) (Table S3). Mutations in NF2 and LATS2 are found in ˜30% of mesotheliomas and mutations in STK11 are found in 18% of lung cancers (Table S3). Previous studies have shown that aberrations in LATS2 and NF2 inactivate the Hippo pathway and overcome crowding-mediated YAP inhibition (Murakami et al., 2011). Despite the oncogenic effect of Hippo pathway mutations, the above studies would predict that the same inactivating mutations in the Hippo pathway genes (NF2, LATS2, STK11) could have an important effect, which can be exploited in chemotherapy: they might be hypersensitive to gemcitabine even in highly crowded conditions and increase the effectiveness of treatment. Indeed, gemcitabine treatment of a broad panel of cancer cell lines harboring Hippo pathway genetic alterations from five diverse cancer types significantly reduced 3D spheroid growth (EC50, <1000 nM) (FIGS. 23A-23B). Interestingly, each of these cell lines has been previously found to be extremely sensitive to gemcitabine in in vitro and some even in mouse xenograft models; however, the mechanism of sensitivity was unclear (Achiwa et al., 2004; Boven et al., 1993; Damaraju et al., 2008; Damaraju et al., 2006; Ikeda et al., 2011; Ratner et al., 2012; Rohde et al., 1998). Furthermore, previous studies have shown that mutations in STK11 (LKB1) in lung cancer cell lines confer sensitivity to gemcitabine while ectopic expression of STK11 causes resistance (Xia et al., 2014; Yang, 2014). STK11 has been identified as an upstream kinase that negatively regulates YAP activity (Mohseni et al., 2014). Increases in the phosphorylation of YAP (3-4-fold) and in the levels of CDA (12-fold) due to cell crowding were observed in lung cancer cells expressing wildtype STK11, while relatively subtle changes (pYAP, 1.5-fold, CDA, 2-fold) were observed in STK11 mutant lung cancer cells (FIGS. 36A-36M). Genetic aberrations in the Hippo pathway can be predictive biomarkers for response to gemcitabine.

Are defects in the Hippo pathway the major cause of gemcitabine sensitivity? It was found that restoration of LATS2 expression in H2052 mesothelioma cells (lacking NF2 and LATS2 expression) causes resistance to gemcitabine in high-density growth (FIG. 14). In crowded conditions, exposure of a low dose (<300 nM) of gemcitabine to parental H2052 cells (LATS2−/−) significantly decreases their viability in response to gemcitabine, as compared to the same cells complemented with wild type LATS2 (FIG. 14). Restoring the levels of LATS2 in H2052 cells caused an increase in the mRNA and proteins levels of ABCG2 and CDA (FIGS. 23C, 36A-36M). LC-MS/MS-based measurement also showed significantly higher amounts of effluxed gemcitabine (˜10-fold) and dFdU (2-3-fold) in the media of H2052 (LATS2) compared with parental H2052 (LATS2−/−) cells (FIG. 23D).

Hippo Pathway Inactivation Sensitizes a Diverse Panel of Human Tumors to Gemcitabine in Mouse Xenografts, and Patient-Derived Xenograft Models

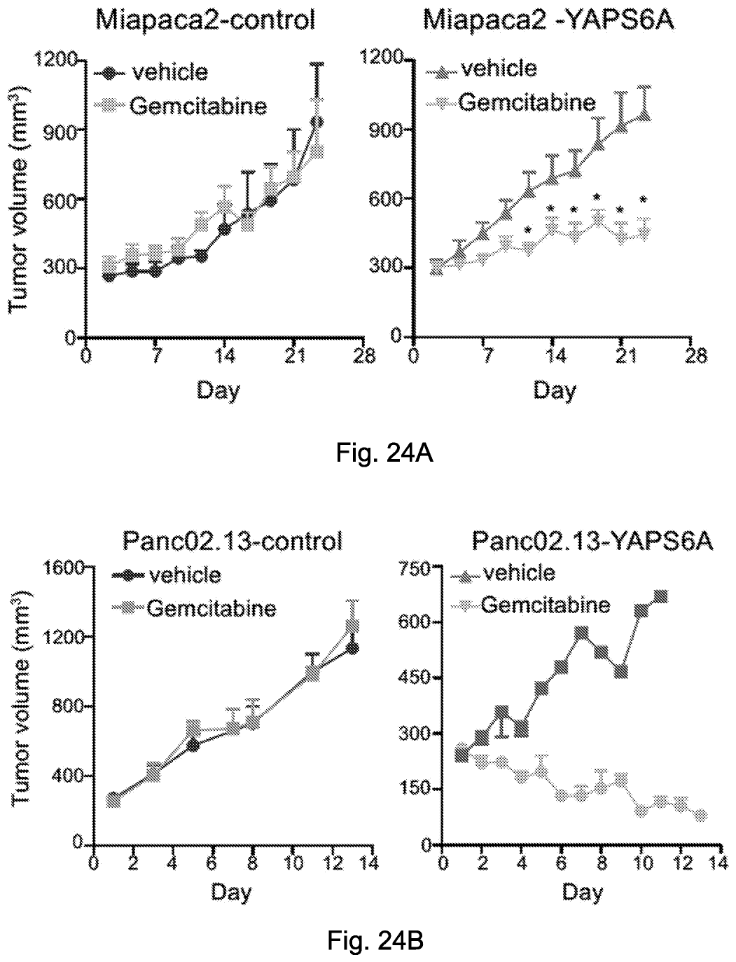

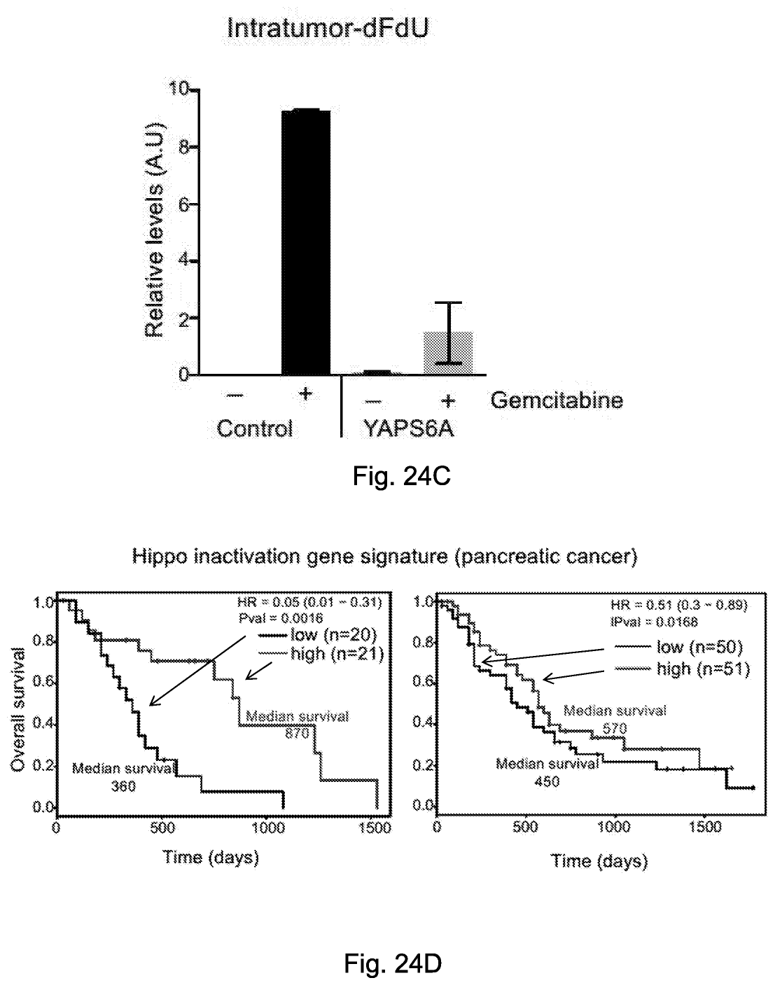

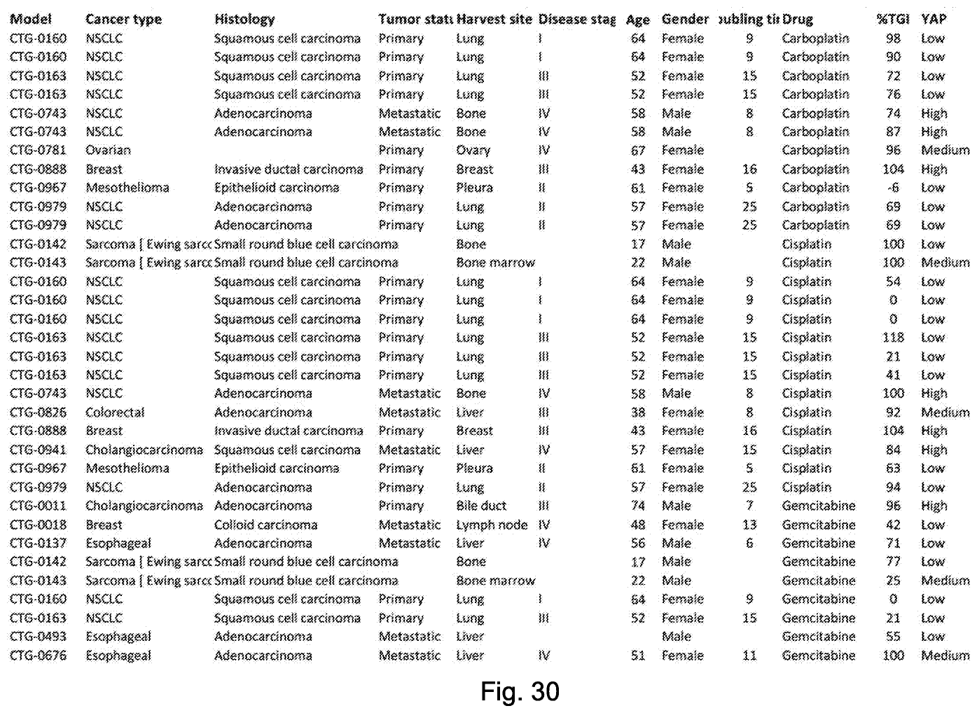

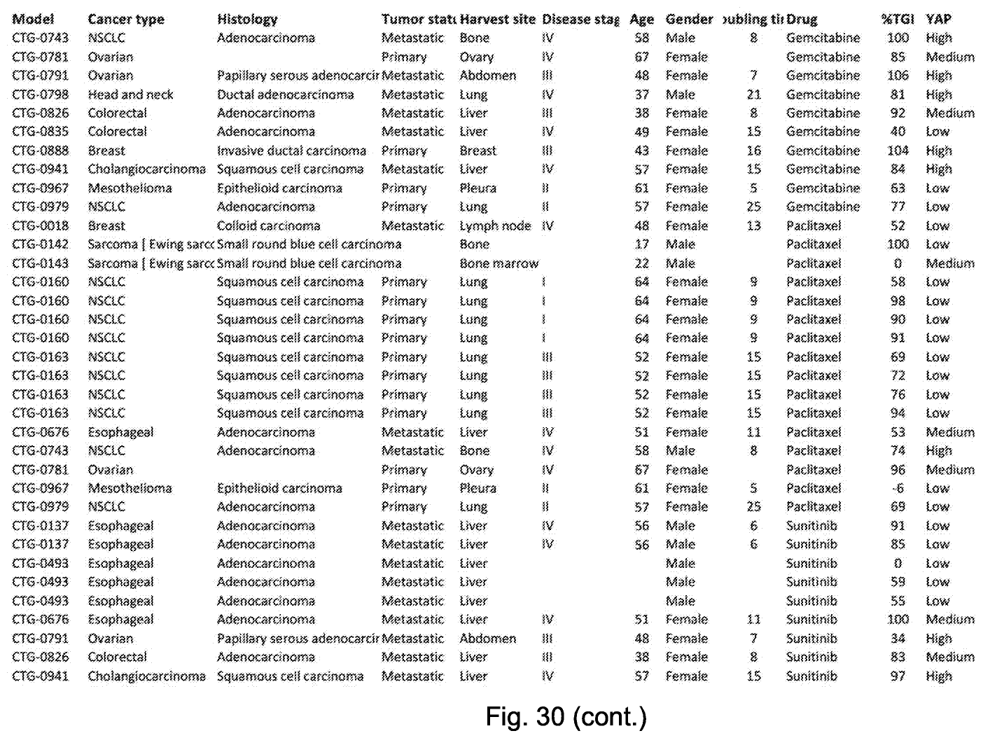

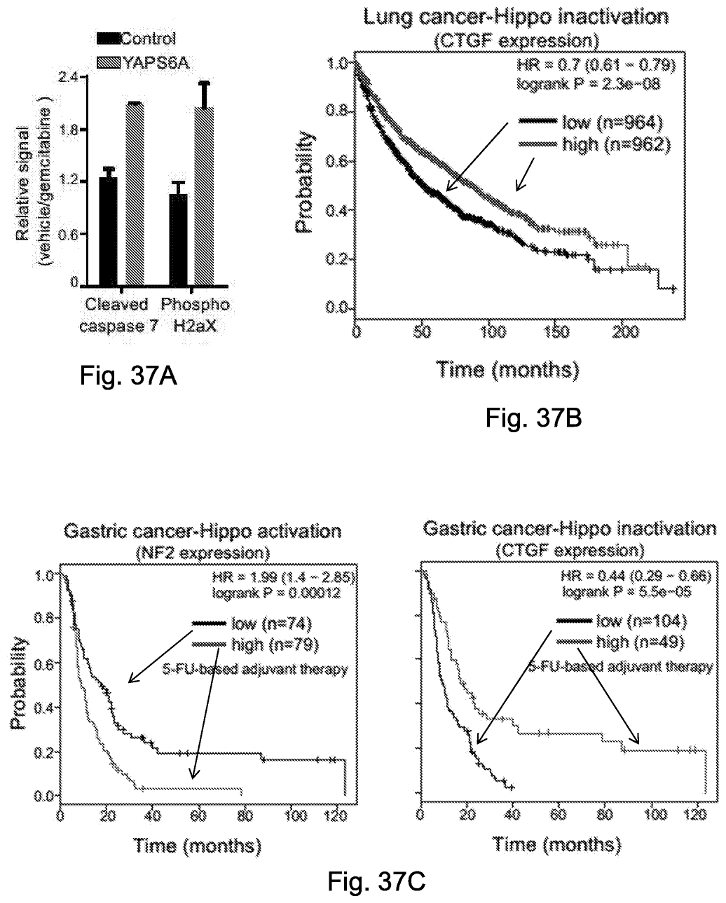

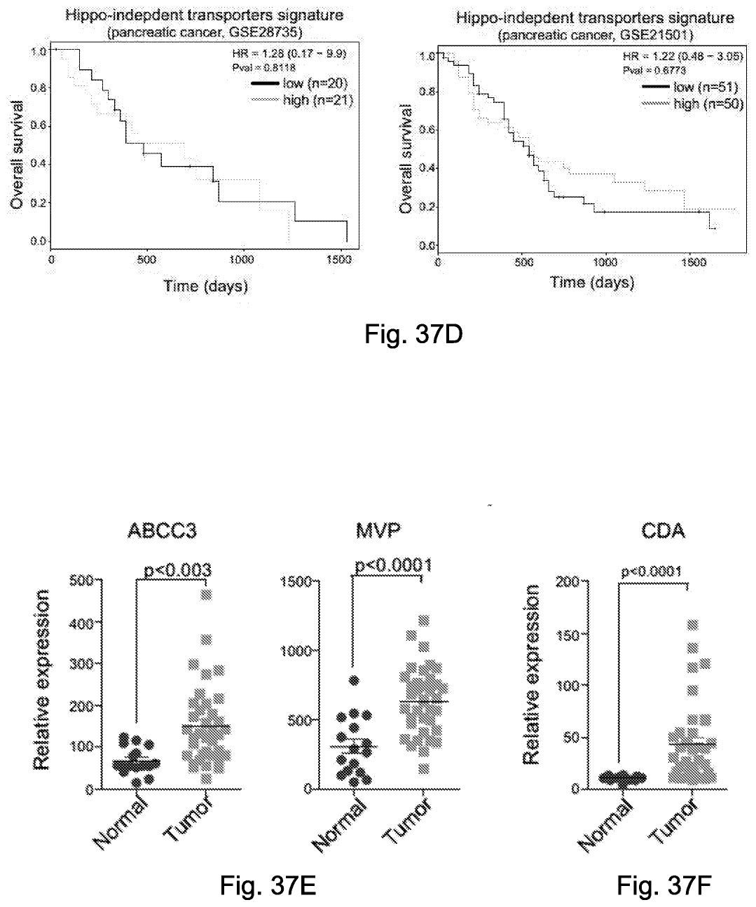

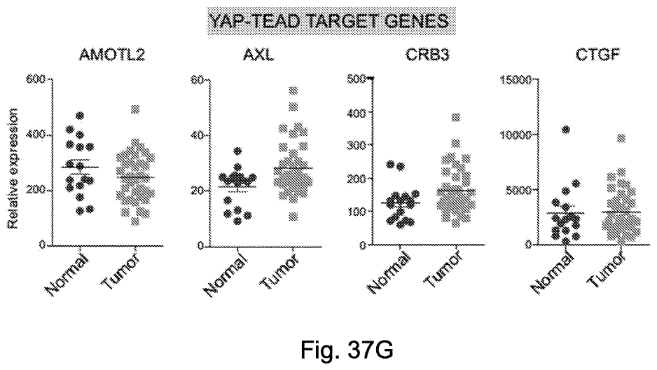

To assess the gemcitabine response to Hippo pathway inactivation in tumors a mouse xenograft model of pancreatic carcinoma cells and patient-derived xenograft (PDX) models from a variety of solid tumors including human cancers from non-small cell lung, esophagus, breast, mesothelium, ovary, colon, head and neck, sarcoma, and cholangiocarcinoma were used (FIG. 30). In mouse xenograft studies, two human pancreatic cancer cell lines (Miapac2 and Panc02.13) expressing GFP or YAPS6A were injected into athymic mice. Both parental or GFP expressing cells grew rapidly, producing palpable tumors in 5-10 days. When the tumors were ˜200 mm³ (as measured using a caliper), mice were randomized into treatment and control groups. The former received i.p. saline injections on alternate days for two weeks, and the latter received gemcitabine (20 mg/kg in Miapaca2-YAPS6A and 50 mg/Kg in Panc02.13-YAPS6A cohorts). Gemcitabine treatment had no affect on the growth of Miapaca2-GFP xenografts as previously observed (Chen et al., 2012) while the growth of Miapaca2-YAPS6A was significantly slowed (FIG. 24A). Similar results were seen in Pan02.13 xenografts where gemcitabine treatment had no affect on the growth of Panc02.13-parental xenografts while gemcitabine treatment of Panc02.13-YAPS6A (50 mg/Kg) led to significant regression in the tumor volume (FIG. 24B). Intra-tumor measurements of the levels of dFdU showed significant reduction (>4-fold, p<0.01) in accumulation of dFdU in Miapca-YAPS6A xenografts compared with parental controls xenografts (FIG. 24C). Consistently, >2-fold induction in apoptosis (measured by levels of cleaved caspase 7 and phosphor-H2aX) was observed in Miapca-YAPS6A xenografts compared with parental controls (FIGS. 37A-37G). These data indicate that “switching-off” the Hippo-YAP pathway overcomes intrinsic drug resistance in PDAC.

It would be natural to next test gemcitabine response in a mouse model of PDA, particularly one that shows a stromal response of connective tissue growth, known as desmoplasia. Unfortunately, the best established PDA mouse models (such as KPC, KrasLSL.G12D/+; p53R172H/+; PdxCretg/+) do not show activation of YAP (the non phosphorylated YAP remains in the nucleus). These tumors would not be expected to be sensitive to gemcitabine. In fact, this mouse model and others are already known to be resistant to gemcitabine (the median survival upon gemcitabine treatment is −15d compared with 10.5d in vehicle control, (Jacobetz et al., 2013)). There may be many interesting features in these mouse PDA models but unfortunately they are not appropriate for studying Hippo and gemcitabine responsiveness.

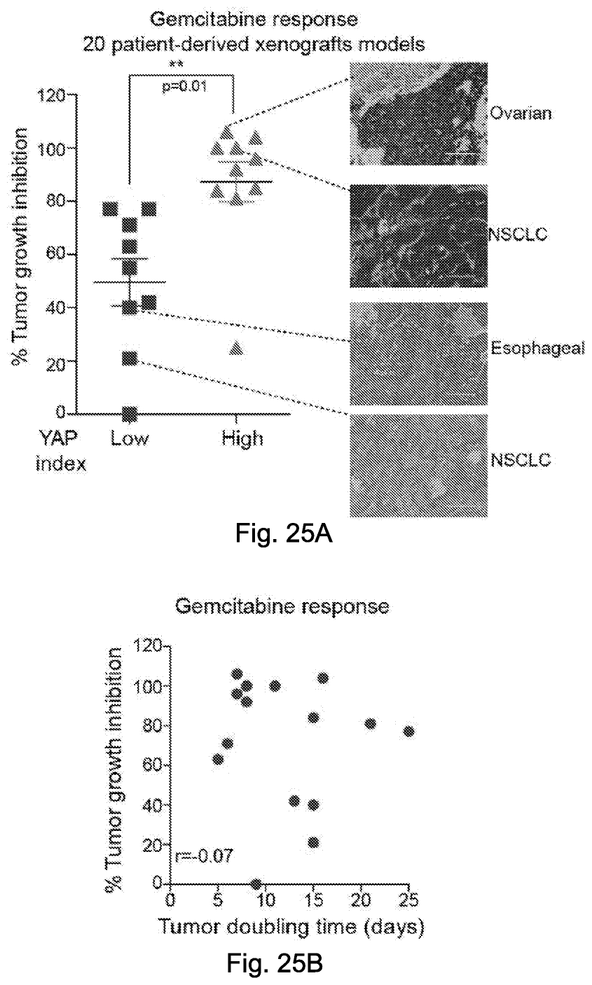

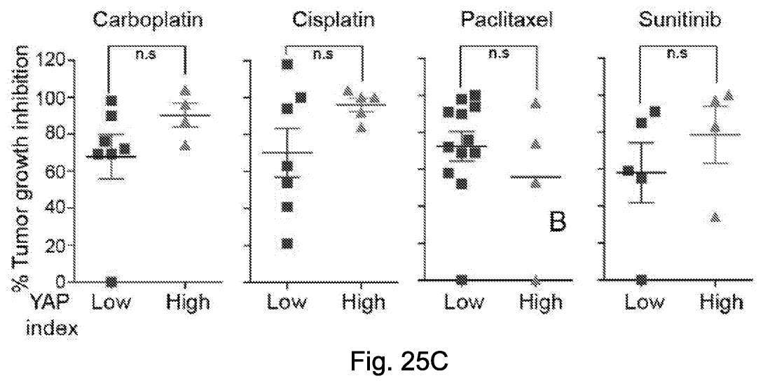

An alternative to an endogenous mouse models for capturing effects of the tumor environment are patient-derived xenograft (PDX) models. PDX models have been shown to retain, the architecture and stromal components of the original tumor and therefore are thought to more accurately represent the complex biochemical and physical interactions between the cancer cells and their microenvironment (Garber, 2009; Tentler et al., 2012). At the cellular level, PDX models also preserve the intra-tumoral heterogeneity, as well as the molecular characteristics of the original cancer, including copy number variants, single-nucleotide polymorphisms, and gene expression profiles (Choi et al., 2014; DeRose et al., 2011). Moreover, studies have found that clinical response of PDXs to therapeutics is correlated with response in patients (Hidalgo et al., 2014). When patient-derived xenograft PDX models were used to assess whether YAP activation sensitizes solid tumors to gemcitabine significant effects were found. The studies were performed in the following manner: Tumor fragments (around 64 mm³) were implanted into the flanks of recipient mice and tumor dimensions recorded with digital calipers. Once tumor implants reached a volume of approximately 200 mm3, dosing with gemcitabine (or vehicle control) began. At the completion of the study completion, the percent tumor growth inhibition (% TGI) was calculated for gemcitabine (G) and the vehicle control (C) using initial (i) and final (f) tumor measurements by the formula: % TGI=[1−(Gf−Gi)/(Cf−Ci)]x100. Tumors with high YAP activity (YAP staining index) showed significantly better response to gemcitabine (˜2-fold difference in % TGI, p=0.01) (FIG. 25A). Notably, there was no correlation between gemcitabine response and tumor doubling time (r=−0.07) (FIG. 25B). In addition, % TGI in response to other cytotoxic drugs including carboplatin and cisplatin was not affected by YAP activity (FIG. 25B). These in vivo data further demonstrate that inactivation of the Hippo-YAP pathway conferd sensitivity to gemcitabine in a diverse panel of cancers.

Gemcitabine is a first line treatment for locally advanced and metastatic pancreatic cancer; therefore, in looking retrospectively at clinical response, it is reasonable to assume that the vast majority of patients were treated with gemcitabine. If Hippo pathway aberrations affect the response of pancreatic cancer to gemcitabine during clinical treatment, this might be revealed by comparing the survival of patients with mutations in the Hippo pathway to those where the Hippo pathway genes were wild type. In two independent studies where exome sequencing was employed it was found herein that high levels of Hippo inactivated genes (AMOTL2, CTGF, AXL, ABCG2, ABCC3, MVP and CRB3) were associated with longer patient survival in pancreatic cancers (FIG. 24D). Specifically, patients with high expression of YAP-TEAD downstream target genes had median survival of 870 days compared with patients with low expression of YAP-TEAD downstream target genes (median survival of 360 days) (FIG. 5D). In lung cancers (˜20% carry STK11 mutations), high expression of CTGF (a YAP-TEAD gene target) correlated with better overall survival (FIGS. 37A-37G), although in this case the data provides no clue to treatment history. Similarly, intrahepatic cholangiocarcinoma patients that express high levels of CTGF have less chance of tumor recurrence and fare better overall survival than those with tumors that lack CTGF expression (Gardini et al., 2005). Gastric cancer patients who received 5-FU-based adjuvant therapy showed better overall survival when the Hippo pathway was inactivated (low NF2 or high CTGF) (FIGS. 37A-37G). Finally, a recent study has also shown that high YAP downstream gene signature correlates with better prognosis in breast cancers (von Eyss et al., 2015). These findings collectively reinforce that Hippo pathway inactivation plays a role in overall survival in certain chemotherapy regimens.

Discussion

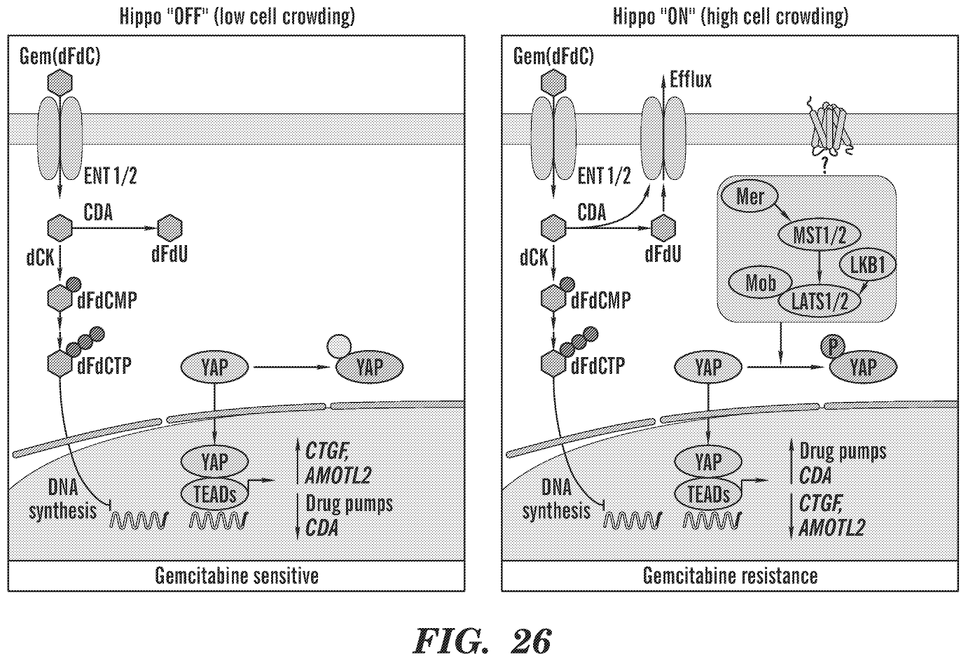

Pancreatic cancer responds poorly to chemotherapy (Oberstein and Olive, 2013); most pancreatic cancer trials have failed, and the current standard-of-care therapy, gemcitabine, has a median overall survival of only six months. (Conroy et al., 2011; Li et al., 2004). Gemcitabine is also used to treat advanced stage lung and breast cancers; however, the determinants of sensitivity and/or resistance to this agent are not fully understood. Comparatively little effort has been directed recently by large drug companies to cytotoxic therapy, possibly because of the belief that there is little to be gained in trying to understand acquired resistance of the current “old fashioned” drugs. Described herein is a previously unknown role of the Hippo-YAP pathway in mediating sensitivity to several chemotherapeutic drugs including gemcitabine (FIG. 26).

At the onset of these experiments with gemcitabine in pancreatic cancer cells, it was surprising to find that there was a large inconsistency in the published results (FIG. 20B, 27). The same cell line in different studies might be reported as sensitive or resistant and this was true in all 15 cell lines tested. In our hands differences in sensitivity depended on the cell density and the effect could be very large (FIG. 20D). Failure to consider cell density is the most likely explanation of this inconsistency and maybe others in large scale pharmacological drug profiling efforts (Haibe-Kains et al., 2013). Today inconsistency is excoriated by critics as another example of the epidemic of irreproducible scientific experiments (Freedman et al., 2015). But it should always be remembered that an alternative and kinder explanation of discrepancies is the extreme sensitivity of some phenomena to experimental conditions, which are often difficult to appreciate. Furthermore, inconsistencies in results have repeatedly been a source of inspiration for discovery, as described herein.

The resolution to the discrepancies concerning gemcitabine is in large part due to the action of the Hippo-YAP pathway, which was activated when cells were grown under crowded conditions (FIG. 5). Inactivation of Hippo-YAP pathway, which naturally occurs under sparse conditions, confers sensitivity to gemcitabine and some other cytotoxic drugs. Experimentally inactivating this pathway by expressing non-phosphorylatable YAP confers sensitivity to crowded cells in 2D and in 3D spheroid culture and also in mouse xenografts (FIGS. 5, 1, 17, 21A-21C, 24A-24D). Most of the interest in the Hippo pathway in cancer is in its role as a tumor suppressor. Paradoxically the present data indicate that upregulating some oncogenes (such as YAPS6A) and downregulating tumor suppressors (such as Retinoblastoma, p53, NF2, or LATS2) can promote the action of certain drugs (Bunz et al., 1999; Herschkowitz et al., 2008; Trere et al., 2009; Zagorski et al., 2007). This appears to be true for gemcitabine and pancreatic cancer, as, described herein, cancer patients carrying a deletion of or inactivating mutation in certain tumor suppressor genes in the Hippo pathway appear to live longer on gemcitabine therapy (FIGS. 24A-24D and 37A-37G).

The present genetic perturbation experiments revealed YAP-TEAD downregulates expression of a suite of multidrug transporters (ABCG2, MVP, ABCC3, ABCC5) as well as cytidine deaminase (CDA), resulting in effectively increasing intracellular availability of gemcitabine (FIGS. 14, 23A-23D). The expression of many of these transporters including ABCG2, ABCC3 and ABCC5 and CDA has been shown to be upregulated in pancreatic carcinoma compared to normal pancreatic tissue (FIGS. 37A-37G) (Konig et al., 2005; Wang et al., 2010). In particular, a recent study has shown that ABCG2 expression regulates gemcitabine response in pancreatic cancer (He et al., 2016). There is some specificity since no correlation was found between overall survival and the levels of Hippo-independent drug transporters in pancreatic cancers (FIGS. 37A-37G). Finally, an increased level of CDA (2-3-fold, p<0.05) was also detected in gastric cancer cells that had acquired resistance to gemcitabine (FIGS. 36A-36M). A recent study has shown that LKB1 (STK11), another activator of the Hippo pathway, enhances chemoresistance to gemcitabine by upregulating CDA in a basal triple negative breast cancer line (Xia et al., 2014). STK11 deletion in mouse Schwann cells led to 6-fold increase in CDA expression levels (FIGS. 36A-36M) (Beirowski et al., 2014). Further, previous studies have shown that poor vascularization of pancreatic tumors limits the intra-tumor availability of gemcitabine (Olive et al., 2009). As described herein, inefficient availability of gemcitabine is an intrinsic property of pancreatic cancer cells and is a major contributor to its drug resistance. Thus, inhibiting Hippo-YAP pathway, which coordinately affects many relevant targets, provides a powerful option for modulating the drug efflux pumps that mediate gemcitabine resistance.

In addition to gemcitabine, several other cytotoxic agents such as antimetabolites and topoisomerase inhibitors are also affected by Hippo-YAP pathway. Therefore, physiological cell crowding seems to mediate the response of several drugs but it is not a completely general condition for all cytotoxic drugs. Without wishing to be bound by theory, it is plausible that the Hippo-YAP sensitization to drugs other than gemcitabine is through modulating intracellular drug levels or drug metabolism. ABCG2 and ABCC3 are known to be broad spectrum drug efflux pumps; substrates of ABCG2 include many drugs which were identified in our screen such as gemcitabine, cladribine, epirubicin, etoposide, imatinib, methotrexate, mitoxantrone, topotecan, teniposide (Cusatis and Sparreboom, 2008) (FIGS. 17, 35A-35H). Alternatively, the intracellular distribution of the drug could be altered by the Hippo pathway, thereby reducing the drug concentration at the site of action. For example, LRP expression is associated with a redistribution of doxorubicin from the nucleus to the cytoplasm without changes in total drug intracellular concentration (Dalton and Scheper, 1999).

The FDA has approved over 100 drugs for use in oncology and there is still a great need to discover more drugs. While drug discovery holds great potential, we can also make important gains through better understanding of how existing drugs work and, perhaps, even more importantly, how they fail (2011). Described herein is how the Hippo pathway plays a role in gemcitabine response and how the status of this pathway can be used as a prognostic marker. Although mutations in the Hippo pathway are relatively uncommon in any given tumor, when specified by organ of origin, in the aggregate they represent a significant frequency of tumor occurrence. Several cell lines harboring genetic alterations with activated YAP in tumors from diverse tissues including lung, ovary, colon and mesothelium. Each was found to be sensitive to gemcitabine in 3D spheroid growth and PDX models (FIGS. 14, 23A-23C, 25A-25C). Due to the relatively low frequency of these mutations, the efficacy of gemcitabine or other drugs would almost certainly have been missed in early trials. Therefore, it could be worth taking into consideration the Hippo pathway status, when considering first line therapy for tumors that harbor Hippo pathway defects. The utility of other drugs that appear to be regulated by the Hippo-YAP pathway should also be considered. With a better understanding of the physiologically adaptive responses of cancer cells to cytotoxic drugs, and the use of molecular markers to identify patients who might therefore qualify as exceptional responders, personalized treatment can be extended to the category of cytotoxic drugs.

Materials and Methods

Cell lines and reagents. Pancreatic cancer cell lines Pancl, Panc02.13, BcPC3, Miapaca2, Panc10.05, Capan2, YAPC, CFPAC1, PATU-8902, PATU-89885, DANG, and ASPC1 cells and mesothelioma cell line H2052 were obtained from American Type Culture Collection (ATCC, Rockville, Md.). Pancl, Miapaca2, PATU-8902, and PATU-89885 were maintained in Dulbecco's Modified Eagle Medium (DMEM) supplemented with 10% (v/v) fetal bovine serum (FBS), 2 mM glutamine, 100 IU/mL penicillin, and 100 μg/mL streptomycin. Panc02.13, BxPC3, Panc10.05, Capan2, YAPC, CFPAC1, DANG, ASPC, and H2052 cells were maintained in Roswell Park Memorial Institute (RPMI) supplemented with 10% (v/v) fetal bovine serum (FBS), 2 mM glutamine, 100 IU/mL penicillin, and 100 μg/mL streptomycin.

Small molecules. Gemcitabine hydrochloride (cat # G-4177) was purchased from LC Labs (Woburn, Mass.). Radiolabeled gemcitabine was purchased from American Radiolabeled Chemicals (St. Louis, Mo.). Irrinotecan (cat # S1198), Paclitaxel (cat # S1150), Docetaxel (cat # S1148), Oxaliplatin (cat # S1224), Etoposide (cat # S1225), Camptothecin (cat # S1288) were purchased from Selleckchem (Houston, Tex.). A set of FDA-approved anticancer drug library consisting of 119 agents was obtained from the Developmental Therapeutics Program, Division of Cancer Treatment and Diagnosis, National Cancer Institute, National Institutes of Health (NIH).

Expression constructs and RNAi. YAP expression construct with serine-to-alanine mutations at S61A, S109A, S127A, S128A, S131A, S163A, S164A, S381A was purchased from Addgene (Plasmid id: 42562). GIPZ Lentiviral shRNAmir clones for human YAP1 or NF2 were purchased from Dharmacon (Lafeyette, Colo.).

Kinetic Cell growth assay. The effect of gemcitabine on pancreatic cancer cell growth was studied using a kinetic cell growth assay. Pancreatic cancer cells were plated on 96-well plates (Essen ImageLock, Essen Instruments, MI, US) at varying densities (2-4X103 for low density or 15-20X103 for high density experiments). Small molecule inhibitors at different doses were added 24 hours after plating and cell confluence was monitored with Incucyte™ Live-Cell Imaging System and software (Essen Instruments). Confluence was observed every hour for 48-144h or until the control (DMSO only) samples reached 100% confluence.

Reverse-Phase Protein Microarray. Cell lysates prepared from various pancreatic cancer cell lines were printed using Aushon 2470 Arrayer™ (Aushon Biosystems). Validation of antibodies, staining, and analysis of array data was performed as described previously (Gujral et al., 2012).

3D spheroid assay. Cancer cell lines were seeded at a 5×10³ cells per well in a 96-well ultra-low adherence plates (Costar) and briefly spun down at 1000 rpm for 5 minutes. After 2 days, cells were treated with small molecule inhibitors at varying concentrations. Growth of spheroids was monitored using live cell imaging every 2-3 hours for 4-7 days in the Incucyte ZOOM™ system (Essen) or as end point assay using CellTiter-Glo™ luminescent cell viability assay (Promega).

Antibodies. Primary antibodies were obtained from the following sources: rabbit phosphor-YAP (S127) (Cell Signaling Technology, Beverly, Mass.; cat. #13008), rabbit anti-YAP (Cell Signaling Technology, Beverly, Mass.; cat. #14074), mouse anti-β-actin (Sigma-Aldrich, Inc., St. Louis, Mo.; cat. # A1978).

Generation of YAPS6A overexpression cell lines. Cell lines (Panc02.13, Panc10.05 or Miapaca2) were transfected with YAPS6A constructs (Addgene plasmid #42562) using Lipofectamine (Invitrogen, Carlsbad, Calif.) following the manufacturer's instructions and 48 hour post-transfection selected in 5-10 μg/ml Blasticidin (InvivoGen, San Diego, Calif.). The clones screened for YAPS6A expression by Western blot. Stable cell lines were maintained in complete medium and 5 μg/ml Blasticidin.

RNA extraction and quantitative real-time PCR. Cells were serum-starved for 24 h and total cellular RNA was isolated using an RNeasy Mini Kit (QIAGEN, Santa Clara, Calif.). mRNA levels for the EMT-related genes were determined using the RT2 Profiler™ qPCR array (SA Biosciences Corporation, Frederick, Md.). Briefly, 1 μg of total RNA was reverse transcribed into first strand cDNA using an RT2 First Strand Kit (SA Biosciences). The resulting cDNA was subjected to qPCR using human gene-specific primers for 75 different genes, and five housekeeping genes (B2M, HPRT1, RPL13A, GAPDH, and ACTB). The qPCR reaction was performed with an initial denaturation step of 10 min at 95° C., followed by 15 s at 95° C. and 60 s at 60° C. for 40 cycles using an Mx3000P™ QPCR system (Stratagene, La Jolla, Calif.).

The mRNA levels of each gene were normalized relative to the mean levels of the five housekeeping genes and compared with the data obtained from unstimulated, serum-starved cells using the 2-ΔΔCt method. According to this method, the normalized level of a mRNA, X, is determined using equation 1:

X=2-Ct(GOI)/2-Ct(CTL) (1)

where Ct is the threshold cycle (the number of the cycle at which an increase in reporter fluorescence above a baseline signal is detected), GOI refers to the gene of interest, and CTL refers to a control housekeeping gene. This method assumes that Ct is inversely proportional to the initial concentration of mRNA and that the amount of product doubles with every cycle.

Protein isolation and quantitative western blotting. Cells were rinsed in Phosphate Buffered Saline (PBS) and lysed in Lysis Buffer (20 mM Tris-HCl, 150 mM NaCl, 1% Triton X-100 (v/v), 2 mM EDTA, pH 7.8 supplemented with 1 mM sodium orthovanadate, 1 mM phenylmethylsulfonyl fluoride (PMSF), 10 μg/mL aprotinin, and 10 μg/mL leupeptin). Protein concentrations were determined using the BCA protein assay (Pierce, Rockford, Ill.) and immunoblotting experiments were performed using standard procedures. For quantitative immunoblots, primary antibodies were detected with IRDye 680-labeled goat-anti-rabbit IgG or IRDye 800-labeled goat-anti-mouse IgG (LI-COR Biosciences, Lincoln, Nebr.) at 1:5000 dilution. Bands were visualized and quantified using an Odyssey™ Infrared Imaging System (LI-COR Biosciences).

Kaplan-Meier Survival Analysis. Kaplan Meier survival curves of pancreatic cancer patients were generated using PROGgene™ using combined signature graph function and Kaplan Meier plotter web-based tools (Gao et al., 2013; Goswami and Nakshatri, 2013; Györffy et al., 2013).

Confocal imaging. Panc02.13 cells were cultured on Lab-Tek II™ chamber glass slides (Nalge Nunc, Naperville, Ill.) or on 24-well glass bottom dishes (MatTek Corporation). Cells were fixed in 4% paraformaldehyde for 15 min at room temperature, washed in PBS, permeabilized with 0.1% Triton X-100, and blocked for 60 min with PBS containing 3% BSA (w/v). Cells were immunostained with the appropriate antibody, following by immunostaining with Alexa Fluor 488-labeled goat-anti-rabbit antibody (Molecular Probes, Eugene, Oreg.). Nuclei were counterstained with Hoescht 33342 (Sigma-Aldrich, St. Louis, Mo.). Fluorescent micrographs were obtained using a Nikon AIR™ point scanning confocal microscope. Individual channels were overlaid using ImageJ™ software (National Institutes of Health, Bethesda, Md.).

Measuring gemcitabine efflux. Panc02.13. cells expressing GFP or YAPS6A plasmid were treated with radiolabeled gemcitabine (0.5 μM) for one hour. Cells were washed twice with PBS and incubated in fresh medium. Medium was collected over the time course of 24 hours and radioactivity was measured using scintillation counter.

Profiling drug transporters. mRNA expression of drug transporters was profiled using Human Drug transporters PCR Array from SA Biosciences (cat # PAHS-070Z) using manufacturer's instructions.

Tumorigenicity in Nude Mice. All in vivo experiments were performed using 6-week-old to 8-week-old athymic nude mice. Mice were maintained in laminar flow rooms with constant temperature and humidity. Miapaca2 or Panc02.13 cells were inoculated subcutaneously (s.c.) into each flank of the mice. Cells (2×10⁶ in suspension) were injected on day 0, and tumor growth was followed every 2 to 3 days by tumor diameter measurements using vernier calipers. Tumor volumes (V) were calculated using the formula: V=AB2/2 (A, axial diameter; B, rotational diameter). When the outgrowths were
200 mm³, mice were divided at random into two groups (control and treated, n=3-8). The treated group received gemcitabine injection or saline control on alternate days (MWF) for 2 weeks.

Patient-derived xenograft (PDX) models. PDX models were established by Champions Oncology (Baltimore, Md.) as described previously (Khor et al., 2015). Drug response to 20 PDX models was obtained from Champions TumorGraft® Database (available on the world wide web at database.championsoncology.com/).

Immunohistochemistry. Human primary tumor tissue slides were obtained from Champions Oncology (Baltimore, Md.). Immunohistochemistry using anti YAP1 antibody (Abcam Cat # ab52771) was performed as previously described (Shi et al., 1999). For negative controls, primary antibody was omitted. The intensity of YAP staining was assessed by an independent pathologist using a four-grade scale: “0” is negative. “0.5” is borderline staining with no significance. “1” is weak staining. “1.5” is weak staining with foci of moderate staining. “2” is moderate staining. “2.5” is moderate staining with foci of strong staining. “3” is homogeneous strong staining. “3.5” is very strong and homogeneous staining with no significant background. “4” is over staining usually with background staining. YAP scoring index was calculated based on staining intensity * % of positive target cells.

Intra-tumor gemcitabine measurements. LC-MS/MS was used to simultaneous quantification of gemcitabine, and it's inactive metabolite dFdU in tumour tissue from a mouse xenograft model of pancreatic cancer as described previously (Bapiro et al., 2011).

### REFERENCES

(2011). Marc Kirschner. Nat Rev Drug Discov 10, 894-894.
(2012). An integrated encyclopedia of DNA elements in the human genome. Nature 489, 57-74. Achiwa, H., Oguri, T., Sato, S., Maeda, H., Niimi, T., and Ueda, R. (2004). Determinants of sensitivity and resistance to gemcitabine: the roles of human equilibrative nucleoside transporter 1 and deoxycytidine kinase in non-small cell lung cancer. Cancer science 95, 753-757. Beirowski, B., Babetto, E., Golden, J. P., Chen, Y. J., Yang, K., Gross, R. W., Patti, G. J., and Milbrandt, J. (2014).
Metabolic regulator LKB1 is crucial for Schwann cell-mediated axon maintenance. Nature neuroscience 17, 1351-1361.
Boven, E., Schipper, H., Erkelens, C., Hatty, S., and Pinedo, H. (1993). The influence of the schedule and the dose of gemcitabine on the anti-tumour efficacy in experimental human cancer. British journal of cancer 68, 52.
Bunz, F., Hwang, P. M., Torrance, C., Waldman, T., Zhang, Y., Dillehay, L., Williams, J., Lengauer, C., Kinzler, K. W., and Vogelstein, B. (1999). Disruption of p53 in human cancer cells alters the responses to therapeutic agents. Journal of Clinical Investigation 104, 263.
Burris, H. r., Moore, M. J., Andersen, J., Green, M. R., Rothenberg, M. L., Modiano, M. R., Cripps, M. C., Portenoy, R. K., Storniolo, A. M., and Tarassoff, P. (1997). Improvements in survival and clinical benefit with gemcitabine as first-line therapy for patients with advanced pancreas cancer: a randomized trial. Journal of clinical oncology 15, 2403-2413.
Camargo, F. D., Gokhale, S., Johnnidis, J. B., Fu, D., Bell, G. W., Jaenisch, R., and Brummelkamp, T. R. (2007). YAP1 increases organ size and expands undifferentiated progenitor cells. Current biology: CB 17, 2054-2060.
Chang, D. K., Grimmond, S. M., Evans, T. J., and Biankin, A. V. (2014) Mining the genomes of exceptional responders. Nature Reviews Cancer 14, 291-292.
Chen, D., Niu, M., Jiao, X., Zhang, K., Liang, J., and Zhang, D. (2012). Inhibition of AKT2 enhances sensitivity to gemcitabine via regulating PUMA and NF-κB signaling pathway in human pancreatic ductal adenocarcinoma. International journal of molecular sciences 13, 1186-1208.
Choi, S. Y., Lin, D., Gout, P. W., Collins, C. C., Xu, Y., and Wang, Y. (2014). Lessons from patient-derived xenografts for better in vitro modeling of human cancer. Advanced drug delivery reviews 79-80, 222-237.
Conroy, T., Desseigne, F., Ychou, M., Bouché, O., Guimbaud, R., Bécouarn, Y., Adenis, A., Raoul, J.-L., Gourgou-Bourgade, S., and de la Fouchardière, C. (2011). FOLFIRINOX versus gemcitabine for metastatic pancreatic cancer. New England Journal of Medicine 364, 1817-1825. Cusatis, G., and Sparreboom, A. (2008). Pharmacogenomic importance of ABCG2.
Dalton, W. S., and Scheper, R. J. (1999). Lung resistance-related protein: determining its role in multidrug resistance. J Natl Cancer Inst 91, 1604-1605.
Damaraju, D., Damaraju, V. L., Brun, M., Mowles, D., Kuzma, M., Berendt, R. C., Sawyer, M. B., and Cass, C. E. (2008). Cytotoxic activities of nucleoside and nucleobase analog drugs in malignant mesothelioma: characterization of a novel nucleobase transport activity. Biochemical pharmacology 75, 1901-1911.
Damaraju, V. L., Damaraju, D., Mowles, D., Berendt, R. C., Sawyer, M. B., and Cass, C. E. (2006). Characterization of nucleoside and nucleobase transporters in a human mesothelial cell line: evaluation of nucleoside and nucleobase antimetabolites for application in malignant mesothelioma. Cancer research 66, 141-141.
DeRose, Y. S., Wang, G., Lin, Y. C., Bernard, P. S., Buys, S. S., Ebbert, M. T., Factor, R., Matsen, C., Milash, B. A., Nelson, E., et al. (2011). Tumor grafts derived from women with breast cancer authentically reflect tumor pathology, growth, metastasis and disease outcomes. Nature medicine 17, 1514-1520. Freedman, L. P., Cockburn, I. M., and Simcoe, T. S. (2015). The Economics of Reproducibility in Preclinical Research. PLoS biology 13, e1002165.
Garber, K. (2009). From human to mouse and back: ‘tumorgraft’ models surge in popularity. J Natl Cancer Inst 101, 6-8.
Gardini, A., Corti, B., Fiorentino, M., Altimari, A., Ercolani, G., Grazi, G., Pinna, A., Grigioni, W., and Grigioni, A. E. (2005). Expression of connective tissue growth factor is a prognostic marker for patients with intrahepatic cholangiocarcinoma. Digestive and liver disease 37, 269-274.
Garnett, M. J., Edelman, E. J., Heidorn, S. J., Greenman, C. D., Dastur, A., Lau, K. W., Greninger, P., Thompson, LR., Luo, X., and Soares, J. (2012). Systematic identification of genomic markers of drug sensitivity in cancer cells. Nature 483, 570-575.
Goswami, C. P., and Nakshatri, H. (2013). PROGgene: gene expression based survival analysis web application for multiple cancers. Journal of clinical bioinformatics 3, 22.
Griffiths, J. (1972). Role of serum, insulin and amino acid concentration in contact inhibition of growth of human cells in culture. Experimental cell research 75, 47-56.
Gujral, T. S., Karp, R. L., Finski, A., Chan, M., Schwartz, P. E., Macbeath, G., and Sorger, P. (2012). Profiling phospho-signaling networks in breast cancer using reverse-phase protein arrays. Oncogene. Hagmann, W., Jesnowski, R., and Lair, J. M. (2010). Interdependence of gemcitabine treatment, transporter expression, and resistance in human pancreatic carcinoma cells. Neoplasia 12, 740-747.
Haibe-Kains, B., El-Hachem, N., Birkbak, N.J., Jin, A. C., Beck, A. H., Aerts, H. J., and Quackenbush, J. (2013). Inconsistency in large pharmacogenomic studies. Nature 504, 389-393. Hao, Y., Chun, A., Cheung, K., Rashidi, B., and Yang, X. (2008). Tumor suppressor LATS1 is a negative regulator of oncogene YAP. Journal of Biological Chemistry 283, 5496-5509.
Harvey, K. F., Zhang, X., and Thomas, D. M. (2013). The Hippo pathway and human cancer. Nature Reviews Cancer 13, 246-257.
Hauswald, S., Duque-Afonso, J., Wagner, M. M., Schertl, F. M., Liibbert, M., Peschel, C., Keller, U., and Licht, T. (2009). Histone deacetylase inhibitors induce a very broad, pleiotropic anticancer drug resistance phenotype in acute myeloid leukemia cells by modulation of multiple ABC transporter genes. Clinical Cancer Research 15, 3705-3715.
He, X., Wang, J., Wei, W., Shi, M., Xin, B., Zhang, T., and Shen, X. (2016). Hypoxia regulates ABCG activity through the activivation of ERK1/2/HIF-lalpha and contributes to chemoresistance in pancreatic cancer cells. Cancer biology & therapy, 1-11.
Herschkowitz, J. I., He, X., Fan, C., and Perou, C. M. (2008). The functional loss of the retinoblastoma tumour suppressor is a common event in basal-like and luminal B breast carcinomas. Breast Cancer Res 10, R75.
Hidalgo, M., Amant, F., Biankin, A. V., Budinska, E., Byrne, A. T., Caldas, C., Clarke, R. B., de Jong, S., Jonkers, J., Maelandsmo, G. M., et al. (2014). Patient-derived xenograft models: an emerging platform for translational cancer research. Cancer discovery 4, 998-1013.
Holohan, C., Van Schaeybroeck, S., Longley, D. B., and Johnston, P. G. (2013). Cancer drug resistance: an evolving paradigm. Nature Reviews Cancer 13, 714-726.
Ikeda, R., Vermeulen, L. C., Lau, E., Jiang, Z., Sachidanandam, K., Yamada, K., and Kolesar, J. M. (2011). Isolation and characterization of gemcitabine-resistant human non-small cell lung cancer A549 cells. International journal of oncology 38, 513-519.
Jacobetz, M. A., Chan, D. S., Neesse, A., Bapiro, T. E., Cook, N., Frese, K. K., Feig, C., Nakagawa, T., Caldwell, M. E., Zecchini, H. I., et al. (2013). Hyaluronan impairs vascular function and drug delivery in a mouse model of pancreatic cancer. Gut 62, 112-120.
König, J., Hartel, M., Nies, A. T., Martignoni, M. E., Guo, J., Büehler, M. W., Friess, H., and Keppler, D. (2005). Expression and localization of human multidrug resistance protein (ABCC) family members in pancreatic carcinoma. International journal of cancer 115, 359-367. Leontieva, O. V., Demidenko, Z. N., and Blagosklonny, M. V. (2014). Contact inhibition and high cell density deactivate the mammalian target of rapamycin pathway, thus suppressing the senescence program. Proceedings of the National Academy of Sciences 111, 8832-8837.
Li, D., Xie, K., Wolff, R., and Abbruzzese, J. L. (2004). Pancreatic cancer. The Lancet 363, 1049-1057.
Liu-Chittenden, Y., Huang, B., Shim, J. S., Chen, Q., Lee, S.-J., Anders, R. A., Liu, J. O., and Pan, D. (2012). Genetic and pharmacological disruption of the TEAD-YAP complex suppresses the oncogenic activity of YAP. Genes & development 26, 1300-1305.
Mohseni, M., Sun, J., Lau, A., Curtis, S., Goldsmith, J., Fox, V. L., Wei, C., Frazier, M., Samson, O., and Wong, K.-K. (2014). A genetic screen identifies an LKB1-MARK signalling axis controlling the Hippo-YAP pathway. Nature cell biology 16, 108-117.
Murakami, H., Mizuno, T., Taniguchi, T., Fujii, M., Ishiguro, F., Fukui, T., Akatsuka, S., Horio, Y., Hida, T., and Kondo, Y. (2011). LATS2 is a tumor suppressor gene of malignant mesothelioma. Cancer research 71, 873-883.
O'CONNOR, R. (2007). The pharmacology of cancer resistance. Anticancer research 27, 1267-1272.
Oberstein, P. E., and Olive, K. P. (2013). Pancreatic cancer: why is it so hard to treat? Therapeutic advances in gastroenterology 6, 321-337.
Olive, K. P., Jacobetz, M. A., Davidson, C. J., Gopinathan, A., McIntyre, D., Honess, D., Madhu, B., Goldgraben, M. A., Caldwell, M. E., and Allard, D. (2009). Inhibition of Hedgehog signaling enhances delivery of chemotherapy in a mouse model of pancreatic cancer. Science 324, 1457-1461.
Perez-Tomas, R. (2006). Multidrug resistance: retrospect and prospects in anti-cancer drug treatment. Current medicinal chemistry 13, 1859-1876.
Polli, J. W., Humphreys, J. E., Harmon, K. A., Castellino, S., O'mara, M. J., Olson, K. L., John-Williams, L. S., Koch, K. M., and Serabjit-Singh, C. J. (2008). The role of efflux and uptake transporters in N-{3-chloro-4-[(3-fluorobenzyl) oxy] phenyl}-6-[5-({[2-(methylsulfonyl) ethyl] amino} methyl)-2-furyl]-4-quinazolinamine (GW572016, lapatinib) disposition and drug interactions. Drug Metabolism and Disposition 36, 695-701.
Quinn, J. E., Kennedy, R. D., Mullan, P. B., Gilmore, P. M., Carty, M., Johnston, P. G., and Harkin, D. P. (2003). BRCA1 functions as a differential modulator of chemotherapy-induced apoptosis. Cancer research 63, 6221-6228.
Ratner, E. S., Keane, F. K., Lindner, R., Tassi, R. A., Paranjape, T., Glasgow, M., Nallur, S., Deng, Y., Lu, L., and Steele, L. (2012). A KRAS variant is a biomarker of poor outcome, platinum chemotherapy resistance and a potential target for therapy in ovarian cancer. Oncogene 31, 4559-4566.
Rohde, D., Hayn, H. K., Blatter, J., and Jakse, G. (1998). The efficacy of 2′,2′-difluorodeoxycytidine (gemcitabine) combined with interferon in human renal cell carcinoma cell lines. International journal of oncology 12, 1361-1366.
Rubin, H. (2005). Magnesium: the missing element in molecular views of cell proliferation control. Bioessays 27, 311-320.
Rudin, D., Li, L., Niu, N., Kalari, K. R., Gilbert, J. A., Ames, M. M., and Wang, L. (2011). Gemcitabine cytotoxicity: interaction of efflux and deamination. Journal of drug metabolism & toxicology 2, 1. Sanford, K. K., Barker, B. E., Woods, M. W., Parshad, R., and Law, L. W. (1967). Search for “indicators” of neoplastic conversion in vitro. Journal of the National Cancer Institute 39, 705-733.
Straussman, R., Morikawa, T., Shee, K., Barzily-Rokni, M., Qian, Z. R., Du, J., Davis, A., Mongare, M. M., Gould, J., and Frederick, D. T. (2012). Tumour micro-environment elicits innate resistance to RAF inhibitors through HGF secretion. Nature 487, 500-504.
Tentler, J. J., Tan, A. C., Weekes, C. D., Jimeno, A., Leong, S., Pitts, T. M., Arcaroli, J. J., Messersmith, W. A., and Eckhardt, S. G. (2012). Patient-derived tumour xenografts as models for oncology drug development. Nature reviews Clinical oncology 9, 338-350.
Trere, D., Brighenti, E., Donati, G., Ceccarelli, C., Santini, D., Taffurelli, M., Montanaro, L., and Derenzini, M. (2009). High prevalence of retinoblastoma protein loss in triple-negative breast cancers and its association with a good prognosis in patients treated with adjuvant chemotherapy. Annals of Oncology 20, 1818-1823.
Veltkamp, S. A., Pluim, D., van Eijndhoven, M. A., Bolijn, M. J., Ong, F. H., Govindarajan, R., Unadkat, J. D., Beijnen, J. H., and Schellens, J. H. (2008). New insights into the pharmacology and cytotoxicity of gemcitabine and 2′, 2′-difluorodeoxyuridine. Molecular cancer therapeutics 7, 2415-2425.
von Eyss, B., Jaenicke, L. A., Kortlever, R. M., Royla, N., Wiese, K. E., Letschert, S., McDuffus, L. A., Sauer, M., Rosenwald, A., Evan, G. I., et al. (2015). A MYC-Driven Change in Mitochondrial Dynamics Limits YAP/TAZ Function in Mammary Epithelial Cells and Breast Cancer. Cancer cell 28, 743-757. Von Hoff, D. D., Ervin, T., Arena, F. P., Chiorean, E. G., Infante, J., Moore, M., Seay, T., Tjulandin, S. A., Ma, W. W., and Saleh, M. N. (2013). Increased survival in pancreatic cancer with nab-paclitaxel plus gemcitabine. New England Journal of Medicine 369, 1691-1703.
Wang, F., Xue, X., Wei, J., An, Y., Yao, J., Cai, H., Wu, J., Dai, C., Qian, Z., and Xu, Z. (2010). hsa-miR-520h downregulates ABCG2 in pancreatic cancer cells to inhibit migration, invasion, and side populations. British journal of cancer 103, 567-574.
Weigelt, B., Reis-Filho, J., and Swanton, C. (2012). Genomic analyses to select patients for adjuvant chemotherapy: trials and tribulations. Annals of Oncology 23, x211-x218.
Xia, C., Ye, F., Hu, X., Li, Z., Jiang, B., Fu, Y., Cheng, X., Shao, Z., and Zhuang, Z. (2014). Liver kinase B1 enhances chemoresistance to gemcitabine in breast cancer MDA-MB-231 cells. Oncology letters 8, 2086-2092.
Yang, C. (2014). LKB1 Deficient Non-small Cell Lung Cancer Cells are Vulnerable to Energy Stress Induced by ATP Depletion.
Zagorski, W. A., Knudsen, E. S., and Reed, M. F. (2007). Retinoblastoma deficiency increases chemosensitivity in lung cancer. Cancer research 67, 8264-8273.
Zhang, N., Bai, H., David, K. K., Dong, J., Zheng, Y., Cai, J., Giovannini, M., Liu, P., Anders, R. A., and Pan, D. (2010). The Merlin/NF2 tumor suppressor functions through the YAP oncoprotein to regulate tissue homeostasis in mammals. Developmental cell 19, 27-38.
Zhang, W., Nandakumar, N., Shi, Y., Manzano, M., Smith, A., Graham, G., Gupta, S., Vietsch, E. E., Laughlin, S. Z., and Wadhwa, M. (2014). Downstream of mutant KRAS, the transcription regulator YAP is essential for neoplastic progression to pancreatic ductal adenocarcinoma. Science signaling 7, ra42.
Zhao, B., Li, L., Lei, Q., and Guan, K.-L. (2010). The Hippo-YAP pathway in organ size control and tumorigenesis: An updated version. Genes & development 24, 862-874.
Zhao, B., Wei, X., Li, W., Udan, R. S., Yang, Q., Kim, J., Xie, J., Ikenoue, T., Yu, J., and Li, L. (2007). Inactivation of YAP oncoprotein by the Hippo pathway is involved in cell contact inhibition and tissue growth control. Genes & development 21, 2747-2761.
Zhao, Y., Lu, H., Yan, A., Yang, Y., Meng, Q., Sun, L., Pang, H., Li, C., Dong, X., and Cai, L. (2013). ABCC3 as a marker for multidrug resistance in non-small cell lung cancer. Scientific reports 3. Zhou, J., Wang, C. Y., Liu, T., Wu, B., Zhou, F., Xiong, J. X., Wu, H. S., Tao, J., Zhao, G., Yang, M., et al. (2008). Persistence of side population cells with high drug efflux capacity in pancreatic cancer. World journal of gastroenterology 14, 925-930.
Bapiro, T. E., Richards, F. M., Goldgraben, M. A., Olive, K. P., Madhu, B., Frese, K. K., Cook, N., Jacobetz, M. A., Smith, D.-M., and Tuveson, D. A. (2011). A novel method for quantification of gemcitabine and its metabolites 2′, 2′-difluorodeoxyuridine and gemcitabine triphosphate in tumour tissue by LC-MS/MS: comparison with 19F NMR spectroscopy. Cancer chemotherapy and pharmacology 68, 1243-1253.
Duxbury, M. S., Ito, H., Zinner, M. J., Ashley, S M., and Whang, E. E. (2004). Inhibition of SRC tyrosine kinase impairs inherent and acquired gemcitabine resistance in human pancreatic adenocarcinoma cells. Clinical cancer research 10, 2307-2318.
Gao, J., Aksoy, B. A., Dogrusoz, U., Dresdner, G., Gross, B., Sumer, S. O., Sun, Y., Jacobsen, A., Sinha, R., Larsson, E., et al. (2013). Integrative analysis of complex cancer genomics and clinical profiles using the cBioPortal. Science signaling 6, p 11.
Giovannetti, E., Mey, V., Danesi, R., Mosca, I., and Del Tacca, M. (2004). Synergistic cytotoxicity and pharmacogenetics of gemcitabine and pemetrexed combination in pancreatic cancer cell lines. Clinical cancer research 10, 2936-2943.
Goswami, C. P., and Nakshatri, H. (2013). PROGgene: gene expression based survival analysis web application for multiple cancers. Journal of clinical bioinformatics 3, 22.
Györffy, B., Surowiak, P., Budczies, J., and Lánczky, A. (2013). Online survival analysis software to assess the prognostic value of biomarkers using transcriptomic data in non-small-cell lung cancer. PloS one 8, e82241.
Hong, S. P., Wen, J., Bang, S., Park, S., and Song, S. Y. (2009). CD44-positive cells are responsible for gemcitabine resistance in pancreatic cancer cells. International journal of cancer 125, 2323-2331.
Huanwen, W., Zhiyong, L., Xiaohua, S., Xinyu, R., Kai, W., and Tonghua, L. (2009). Intrinsic chemoresistance to gemcitabine is associated with constitutive and laminin-induced phosphorylation of FAK in pancreatic cancer cell lines. Mol Cancer 8, 21.
Humbert, M., Castéran, N., Letard, S., Hanssens, K., Iovanna, J., Finetti, P., Bertucci, F., Bader, T., Mansfield, C. D., and Moussy, A. (2010). Masitinib combined with standard gemcitabine chemotherapy: in vitro and in vivo studies in human pancreatic tumour cell lines and ectopic mouse model. PLoS One 5, e9430.
Khor, T. O., Zvi, I. B., Katz, A., Vasquez-Dunddel, D., Sloma, I., Ciznadija, D., Sidransky, D., and Paz, K. (2015). A patient-centric repository of PDX models for translational oncology research. Cancer research 75, 3219-3219.
Modrak, D. E., Leon, E., Goldenberg, D. M., and Gold, D. V. (2009). Ceramide regulates gemcitabine-induced senescence and apoptosis in human pancreatic cancer cell lines. Molecular Cancer Research 7, 890-896.
Mori-Iwamoto, S., Kuramitsu, Y., Ryozawa, S., Taba, K., Fujimoto, M., Okita, K., Nakamura, K., and Sakaida, I. (2008). A proteomic profiling of gemcitabine resistance in pancreatic cancer cell lines. Mol Med Rep 1, 429-434.
Parsels, L. A., Morgan, M. A., Tanska, D. M., Parsels, J. D., Palmer, B. D., Booth, R. J., Denny, W. A., Canman, C. E., Kraker, A. J., and Lawrence, T. S. (2009). Gemcitabine sensitization by checkpoint kinase 1 inhibition correlates with inhibition of a Rad51 DNA damage response in pancreatic cancer cells. Molecular cancer therapeutics 8, 45-54.
Shi, S.-R., Guo, J., Cote, R. J., Young, L. L., Hawes, D., Shi, Y., Thu, S., and Taylor, C. R. (1999). Sensitivity and detection efficiency of a novel two-step detection system (PowerVision) for immunohistochemistry. Applied Immunohistochemistry & Molecular Morphology 7, 201.
Shi, X., Liu, S., Kleeff, J., rg, o., Friess, H., and Buchler, M. W. (2002). Acquired resistance of pancreatic cancer cells towards 5-Fiuorouracil and gemcitabine is associated with altered expression of apoptosis-regulating genes. Oncology 62, 354-362.
Yang, W., Soares, J., Greninger, P., Edelman, E. J., Lightfoot, H., Forbes, S., Binda I, N., Beare, D., Smith, J. A., and Thompson, I. R. (2013). Genomics of Drug Sensitivity in Cancer (GDSC): a resource for therapeutic biomarker discovery in cancer cells. Nucleic acids research 41, D955-D961.

1. A method of treating cancer, the method comprising administering a chemotherapeutic selected from the group consisting of:
an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor;
to a subject having cancer cells determined to have:
a. a deletion, a truncation or inactivating mutation in FAT4; LATS1; LATS2; STK11; or NF2;
b. decreased expression of FAT4; LATS1; LATS2; STK11; or NF2 relative to a reference;
c. increased expression of YAP; CTGF; AREG; AMOTL2; AXL; or BIRC5 relative to a reference;
d. decreased phosphorylation of YAP relative to a reference; or
e. increased nuclear localization of YAP relative to a reference.

2. The method of claim 1, wherein the antimetabolite or nucleoside analog is selected from the group consisting of:
gemcitabine; 5-FU; cladribine; cytarabine; tioguanine; mercaptopurine; and clofarabine.

3. The method of claim 1, wherein the antifolate is methotrexate.

4. The method of claim 1, wherein the topoisomerase I inhibitor is camptothecin, topotecan, or irinotecan or the topoisomerase II inhibitor is selected from the group consisting of:
epirubicin; daunorubicin; doxorubicin; valrubicin; teniposide; etopiside; and mitoxantrone.

5. (canceled)

6. The method of claim 1, wherein the anthracycline is selected from the group consisting of:
epirubicin; daunorubicin; doxorubicin; and valrubicin.

7. The method of claim 1, wherein the tubulin modulator is ixabepilone.

8. The method of claim 1, wherein the Src family kinase inhibitor or BCR-Abl kinase inhibitor is imatinib.

9. The method of claim 1, wherein the DNA cross-linking agent is mitomycin.

10. A method of treating cancer, the method comprising administering a chemotherapeutic selected from the group consisting of:
an antimetabolite; an anthracycline; an anthracycline topoisomerase II inhibitor; a proteasome inhibitor; an mTOR inhibitor; an RNA synthesis inhibitor; a peptide synthesis inhibitor; an alkylating agent; an antiandrogen; a Src family kinase inhibitor; a BCR-Abl kinase inhibitor; a MEK inhibitor; and a kinase inhibitor; to a subject having cancer cells determined not to have:
a. a deletion, a truncation, or inactivating mutation in FAT4; LATS1; LATS2; STK11; or NF2;
b. decreased expression of FAT4; LATS1; LATS2; STK11; or NF2 relative to a reference;
c. increased expression of YAP; CTGF; AREG; AMOTL2; AXL; or BIRC5 relative to a reference;
d. decreased phosphorylation of YAP relative to a reference; or
e. increased nuclear localization of YAP relative to a reference.

11. The method of claim 10, wherein the anthracycline toposisomerase II inhibitor is selected from the group consisting of:
daunorubicin; doxorubicin; epirubicin; and valrubicin
or the anthracycline is selected from the group consisting of:
daunorubicin, doxorubicins; epirubicin; and valrubicin.

12. (canceled)

13. The method of claim 10, wherein the proteasome inhibitor is carfilzomib or bortezomib.

14. The method of claim 10, wherein the mTOR inhibitor is everolimus.

15. The method of claim 10, wherein the RNA synthesis inhibitor is triethylenemelamine, dactinomycin, or plicamycin.

16. The method of claim 10, wherein the kinase inhibitor is ponatinib or trametinib or the Src family kinase inhibitor or BCR-Abl kinase inhibitor is ponatinib, or the MEK inhibitor is trametinib.

17. (canceled)

18. (canceled)

19. The method of claim 10, wherein the antiandrogen is enzalutamide.

20. The method of claim 10, wherein the peptide synthesis inhibitor is omacetaxine mepesuccinate.

21. The method of claim 1, wherein the mutation in FAT4; LATS1; LATS2; STK11; or NF2 is selected from Table 2.

22. The method of claim 1, wherein the method further comprises a step of detecting the presence of one or more of:
a. a deletion, a truncation, or inactivating mutation in FAT4; LATS1; LATS2; STK11; or NF2;
b. decreased expression of FAT4; LATS1; LATS2; STK11; or NF2 relative to a reference;
c. increased expression of YAP; CTGF; AREG; AMOTL2; AXL; or BIRC5 relative to a reference;
d. decreased phosphorylation of YAP relative to a reference; or
e. increased nuclear localization of YAP relative to a reference.

23.-35. (canceled)

36. The method of claim 1, wherein the cancer is pancreatic cancer; pancreatic ductal adenocarcinoma; metastatic breast cancer; breast cancer; bladder cancer; small cell lung cancer; lung cancer; ovarian cancer;
stomach cancer; uterine cancer; mesothelioma; adenoid cystic carcinoma; lymphoid neoplasm; kidney cancer; colorectal cancer; adenoid cystic carcinoma; prostate cancer;
cervical cancer; head and neck cancer; and glioblastoma.

37. An assay comprising:
detecting, in a test sample obtained from a subject in need of treatment for cancer;
i. a deletion, a truncation or inactivating mutation in FAT4; LATS1; LATS2; STK11; or NF2;
ii. decreased expression of FAT4; LATS1; LATS2; STK11; or NF2 relative to a reference;
iii. increased expression of YAP; CTGF; AREG; AMOTL2; AXL; or BIRC5 relative to a reference;
iv. decreased phosphorylation of YAP relative to a reference; or
v. increased nuclear localization of YAP relative to a reference.
wherein the presence of any of i.-v. indicates the subject is more likely to respond to treatment with a chemotherapeutic selected from the group consisting of:
an antimetabolite; a nucleoside analog; an antifolate; a topoisomerase I inhibitor; a topoisomerase II inhibitor; an anthracycline; a tubulin modulator; a DNA cross-linking agent; a Src family kinase inhibitor; and a BCR-Abl kinase inhibitor.

38.-103. (canceled)


SEQUENCE LISTING (12 sequences)

SEQ ID NO: 1 - DNA, 16120 bp, Homo sapiens
atggacttagcaccagacagggctactggccgcccgtggctcccgttgcacactctatcagtatctcagctccttcgagtgttttggctactgtcattgcttccggggcaggcctgggtccacggggccgagccgcgccaggtgttccaagtgctggaagagcaacctccaggcactctggtaggcaccatccagacgcgccccggcttcacctacaggctcagcgaaagccacgccctgtttgccataaacagtagcaccggagccctgtacaccacctccaccatcgaccgcgagagcctgcccagcgacgtgatcaacctggtggtcctttccagcgcgcccacctaccccaccgaagtgcgagtgctggtgcgggacctcaatgacaacgcccccgttttcccggacccctctatcgtggtcactttcaaggaagacagtagcagcggacgccaagtcatcttagacaccgccaccgactcggacatcggctcaaacggtgtggaccaccgctcctaccgcatcatccgcggcaatgaggcggggcgcttccgtctggacatcaccctgaacccgagcggcgagggagcgttcctgcatctggtgtccaagggcggactggaccgtgaggtcactccgcagtaccagctcctggttgaggtggaggacaagggtgagcctaagcggcggggctaccttcaggtaaacgtgactgtgcaagacattaatgacaaccccccggtttttggcagttctcactaccaggcgggggtgcctgaggacgcggttgtgggttccagcgtcctccaggtggcggcggcggacgcggacgagggcaccaacgcggacatccgctatcgcctgcaggacgaggggacccccttccaaatggaccctgagacgggacttatcacggtgcgggagcccctggacttcgaagctcggcgccaatactcgcttacggtgcaggcgatggacagaggcgtgccttccctcactgggcgcgccgaggcgctgattcagctgctggacgtgaatgacaatgacccggtagtgaagttccgctacttcccggccacctcgcgctacgcctcggtagatgagaatgctcaagtgggcaccgtggtggctctgctcaccgtgacggacgcagattctcccgcggccaacgggaacatctccgtgcaaattctcgggggcaatgagcagcgccactttgaagtgcaaagcagcaaagtgccgaacctgagcctaatcaaggtggccagcgccttggaccgcgagcgcatcccttcctacaacctcacagtttccgtctctgataactacggggcgccccctggcgcagcagtccaggcgcgctcttctgtggcaagcctggtgatttttgttaatgacatcaatgaccatcctcctgtcttttcacagcaagtgtacagagtgaacctgagcgaggaggcgcctccgggaagctatgtgagtgggatatctgccactgatggcgactctggtctcaatgctaatctgcgttacagcattgtctctggcaatggactgggatggttccatatcagtgaacatagcggcctcgtgaccactgggtcctctgggggcctggaccgtgaacttgcttcccagattgttctgaatataagtgcccgggaccagggagttcaccccaaggtgtcctatgcccagcttgtagtaactctcctagatgtgaatgatgaaaagccagtatttagccagccagaagggtatgatgtgtctgtggttgagaatgccccaacagggacagaactgttgatgctcagggcaactgacggggacctgggtgacaacggaacagtgcgcttctccttacaagaggcagagactgaccggaggtccttccgtctggatcctgtgtctgggaggttgagtactatttcctccttggacagagaagagcaagccttctactccctgttggttctggccacagatctgggctcccctccccagtcatcaatggctcgcataaatgtgagtcttctggatataaatgataacagccctgtcttctacccggtccaatactttgctcacattaaggagaatgagcctggaggtagctacatcaccactgtgtctgccactgacccagacttgggtaccaatggtactgtcaaatatagcatatctgctggggacaggtctcggtttcaggtcaatgctcagagtggggttatttctacaagaatggccctagacagagaagaaaaaacagcttatcagttgcaaatagtagctactgatggtggcaatttacaatctcccaaccaggcaatagtaaccatcactgtattggacactcaagacaacccacctgtattcagtcaggttgcctacagctttgtggtttttgagaacgtggcgctgggatatcatgtgggtagtgtgtctgcatccaccatggatctcaattccaacatcagttatctcattactactggggatcagaaaggtatgtttgctatcaaccaggtcactgggcagcttaccacagcaaatgtgattgatagagaagagcaatccttttatcagctgaaggtagtggccagtgggggcacagtgactggagacactatggttaacataacagttaaggatttgaatgacaactctccccatttccttcaggcaatagagagtgtaaatgtggtggagaattggcaggcaggtcacagcattttccaggccaaagctgtggaccctgatgaaggtgtcaatggcatggtactctatagtctgaagcaaaaccccaagaacctgtttgctatcaatgaaaagaatggcactattagtctgcttgggcccctggatgttcatgctggctcctaccaaatagagatcttggcatctgacatgggtgtcccacagctctcctctagtgtcatcttaacagtttatgtccatgatgtaaatgacaattcaccagtgtttgaccaactctcttatgaagtcaccctttctgagtcagaacctgtgaattctcgattctttaaagtacaagcttctgataaggattcaggagcaaatggtgaaattgcatacaccattgctgaaggaaatacaggggatgcttttggcatattcccagatggtcaattgtatataaaaagtgaactggaccgtgaacttcaagacagatatgttttaatggttgttgcttctgacagagcagtggaaccccttagtgctactgtgaatgttactgtaattttagaagatgtaaatgataacagacctctttttaacagtaccaattacacattttacttcgaagaagagcagagggctgggtcgtttgtgggcaaagtaagtgctgtagataaagactttgggccaaatggagaagtaaggtattcttttgaaatggtgcagccagattttgagttgcatgccatcagtggggaaattacaaatactcatcagtttgacagggagtctcttatgaggcggagagggactgctgtgtttagctttacagtcatagcaacagatcaggggatccctcagcctctcaaggatcaggccactgtacatgtttacatgaaggatataaatgataatgctcccaaatttttaaaagacttttaccaagctacaatatcagaatcagcagccaatctgacacaagtgttaagagtatctgcctcagatgttgatgaaggtaataatggacttattcactattctataataaaaggaaatgaagaaagacagtttgctatagacagtacctctggtcaggtaacactaattggcaaattagactatgaagcaacacctgcctattcccttgtaattcaagcagtggattcagggacaatccccctcaattcaacgtgtactttaaatattgatattttagatgaaaatgacaataccccttctttccctaaatcaacactctttgttgatgttttggaaaacatgagaattggtgaactcgtgtcctctgttactgcaactgattccgattcaggtgacaatgctgatttatattacagtattactgggactaacaaccacggaacttttagcattagcccaaacactgggagtatttttcttgccaaaaaactggactttgaaacacagtctttgtataaattaaatataacagcaaaagaccaaggaagacctcctcgttcatctacaatgtcagtggttattcacgtgagggactttaatgacaatcctcctagctttcctcctggagatattttcaagtctattgttgagaacattcccatcggtacatctgtcatttcagtgactgcacatgaccctgatgcagacattaatggtcaactatcctacacaatcattcaacagatgccaagaggcaaccactttaccatagatgaagtcaaagggactatatatactaatgctgaaatagatcgggaatttgctaatctctttgagttgactgtaaaagccaatgatcaagctgtgccaatagaaactagacggtatgctttgaagaacgtgaccattttggttacagacctcaatgacaatgtcccaatgtttatatcacaaaacgcccttgctgcagacccatcagctgtgattggttccgttctgacaacaattatggctgctgacccagatgaaggtgctaatggagaaatagagtatgagatcatcaatggggacacagacaccttcattgttgatcgttatagtggagacctgagagtggcttcagcgttggtgccttcacagttgatctacaatctcatagtttcagcaacagaccttgggcctgaaaggaggaaatcgaccactgaattgaccatcattcttcagggccttgatggacctgtttttactcaacccaaatatataactattttgaaggaaggagaacccattggcacaaacgtgatatcaatagaagcagctagccccagaggatctgaggccccagtggagtattatattgtttcagttcgttgtgaagaaaaaactgttggacgcctctttactattggacgacatactggtataattcagaccgcagccattctggaccgggagcaaggagcatgtctttacctggtggatgtttatgccatagaaaaatcaactgcttttcccagaacacagagagcagaggtagaaataacacttcaggatatcaatgacaatccaccagtatttccaacggacatgctggatctcacggtagaggagaacattggagatggctctaagattatgcagctgacagccatggatgctgatgagggtgcaaatgctctcgtcacatacactatcattagtggagctgatgatagttttcgcatcgacccagaatccggagatctgatagcaaccaggcggttggacagggaacgccgctccaaatattcactgctagttcgtgctgatgatggtcttcagtcctcggatatgagaattaatatcactgtcagtgatgtgaatgaccatacacccaaattttccagacccgtgtactcttttgacattcctgaggacacaatccctggttctttggtagcagccattttagccacggatgatgactctggtgtgaatggagaaattacatatattgtgaatgaagatgatgaagatggcatctttttcctgaatcctattactggggtctttaatttgactcgattattagattatgaagtacagcaatattatatcctcactgttcgagcagaagatggtgggggacaatttactaccatcagagtttatttcaatattctagatgtaaatgataatccacctattttcagcttgaattcatacagcacatctttaatggagaatctacctgtgggatctactgttcttgtgtttaatgttactgatgcagatgatggcatcaactctcaattgacttatagcattgcttcaggtgatagccttgggcagtttactgttgacaagaatggtgtactcaaagtcctaaaagctttggatcgggaaagtcagtccttctacaacttggttgttcaagtgcatgacctgccacagattccagcctccagattcacaagcactgctcaagtctccattattttgttggatgtaaatgataacccaccgacatttctttcccctaaattgacatacattccagaaaatacacctattgatactgttgttttcaaagctcaagcaactgacccagatagtggcccaaacagctatattgagtacactctgctgaaccctttgggaaacaagttcagtattgggaccattgatggtgaagtgaggctcactggagaactggacagagaagaagtttctaattatactctaacagtggtggctacagacaaaggtcaaccatctctctcttcatctacagaggttgtagttatggtacttgacatcaatgataacaaccccatctttgcacaagctttgtataaagtggagattaatgaaaacacacttactggaacagatataatacaagtgttcgcagcagatggagatgaaggcacaaatggacaggttcgctatggcattgttaatggtaataccaatcaggaatttcggatagactctgtcacaggtgccatcactgtcgctaaacctttggatagagaaaagacccctacctaccatttaactgttcaggcaacagatcgaggcagcacacccagaactgatacctccacggtcagcattgttctactggatattaatgactttgttcctgtatttgagctatctccatattctgtaaatgtccctgagaatttagggacactacccagaacaattcttcaggtggtggcaagagatgatgatcgaggatctaacagcaaactctcatatgttctgtttggtggtaatgaagacaatgcttttactctctcagccagtggagaacttggagtaacacagagtctggatcgggaaacaaaagagcgctttgtcttaatgattacagctacagattcaggatcccctgccttgactggaactggaacaatcaacgtcatagtagatgatgtcaatgacaatgtccccacatttgccagtaaagcgtatttcacaacaattcctgaggatgcaccaactggaacagatgttttattggtaaatgcctcagatgctgatgcttcaaagaatgcagttataagttataggatcatcggtggaaactctcagttcacgatcaacccatcgacaggacaaatcatcaccagcgcattgttagatagggaaacaaaagataattatactttggtagtggtctgcagtgatgcgggatccccagagcctctttccagttccaccagtgtgcttgtcactgtgactgatgtcaatgacaatccaccaagatttcagcatcacccatatgtcactcacatcccatctcctactcttccaggttcctttgtctttgcggttacagtcacagatgctgatattggaccaaattctgaactgcattattctctttcgggtagaaattctgaaaaatttcacattgacccactgaggggagccattatggccgccggaccactaaacggagcttcagaagtgacattttctgtgcatgtaaaagatggtggctcatttccaaagacagattctacaacagtgactgttagattcgtgaataaggccgatttccctaaagtgagagccaaagaacaaacgttcatgtttcctgaaaaccaaccagtcagctctcttgtcaccaccatcacaggatcctctttaagaggagaacctatgtcatattatatcgcaagtgggaatcttggcaatactttccagattgatcagttaacagggcaggtgtctattagtcaacctctggattttgaaaagatacaaaaatatgttgtatggatagaggccagagacggtggtttccctcctttctcctcttacgagaaacttgatataacagtattagatgtcaatgataatgccccaatttttaaggaagacccatttatatctgaaatattggaaaacctttcccctcgaaaaatacttactgtttcggcaatggacaaggacagtggacccaatggacagttagattatgaaattgttaatggcaacatggaaaatagtttcagtatcaatcatgctactggtgaaattagaagcgttagacctttggacagggaaaaagtatctcattatgtcctaaccataaaatcatcagacaaagggtccccgtctcagagtacttcagtaaaagtcatgattaacattttagatgaaaatgataatgcccctaggttttctcagatatttagtgcccatgttcctgaaaattcccccttaggatacacagttacccgtgtcacaacttctgatgaagacattgggatcaatgcaattagtagatattctataatggatgcaagtcttccatttacaattaatcccagcacaggggatattgtcataagcagacctttaaatagggaagatacagaccgttacagaattcgagtttccgcacatgattctgggtggactgtaagtacagatgtcacaatatttgtgacagacatcaatgacaatgctccaagatttagcagaacttcctattatttagattgccctgaacttactgagattggctccaaagtaactcaggtatttgcaacagatcctgatgagggatcaaatggacaagtgttttatttcataaaatcccaatcagaatatttcaggattaatgccaccactggagagattttcaataaacagatcttaaaataccaaaatgtcactggcttcagtaatgtgaatatcaacaggcatagttttatagtgacatcttcagatcgaggtaaaccttccttaattagtgagacaacagttaccatcaatatagtggacagtaatgacaatgcacctcaatttcttaaaagtaaatatttcactccagtcaccaaaaatgttaaggttggtacgaagttaatcagagttacagcaatagatgacaaagattttggactgaattcagaagtggagtatttcatttctaatgataaccatttaggaaaatttaagttggacaatgatacggggtggatttcagtagcatcctccctgatttctgacttgaaccaaaacttttttatcacagtcactgcaaaggataagggaaaccctccactttcttcccaagcaactgttcacataactgtcactgaggaaaactaccatacacctgaattctctcaaagccacatgagtgcaaccatccctgagagccatagcattgggtccattgtcagaactgtttctgcaagagatagagatgcagcgatgaatggcttgattaagtacagcatttcttcaggaaatgaagaaggcatttttgcaatcaattcttctacaggtatattaacactagccaaagctcttgattatgagctatgccagaaacacgaaatgacgattagtgctatagatggaggatgggttgcaagaactggttactgcagtgtgaccgtaaatgtgattgatgtgaatgataattctccagtattcctctctgatgactatttccctactgttttggaaaatgccccaagtggaacaacagttatccacctaaatgcaacagatgctgactctggaacaaatgctgtgattgcgtatactgtacagtcatctgacagtgacctctttgtcattgaccctaacacaggagtcataaccactcaaggcttcttggattttgaaaccaagcagagctaccatcttactgtgaaagccttcaatgtccccgatgaggaaaggtgtagctttgccactgttaatatacaattaaaagggacaaatgaatatgtgccccgttttgtttccaaactttactattttgaaatctcagaagcagctcctaaaggtactattgttggagaagtgtttgctagcgaccgtgatttgggcactgatggggaggtacactatttgatttttggtaatagtcgaaagaagggtttccagatcaataagaagactggacagatttatgtttctggaattcttgatcgagaaaaagaagaaagggtgtctttgaaggtattggccaagaactttggcagcattagaggtgcagatatagatgaggtcactgtaaatgtcaccgtgcttgatgcaaatgacccacccatttttactctaaacatctacagtgtgcagatcagtgaaggggtcccaataggaactcatgtgacctttgtcagtgcctttgactcagactccatccccagctggagcaggttttcttacttcatcggatcagggaatgaaaatggtgccttttctattaatccgcagacaggacagatcaccgttactgcagaattagatcgagaaacccttcccatctataatctctcagttttggctgttgattcagggaccccctcagctacaggtagtgcctctttattagtcaccctggaagatataaatgataacgggcccatgctgactgtcagtgaaggagaagtcatggaaaacaaacggccaggcactttggtgatgacccttcagtccactgaccctgatctccctccaaatcaaggtccctttacttattacttgctgagcacaggtcctgccaccagttatttcagtctgagcactgctggagttctgagcacaaccagagagattgacagagagcagattgcagacttctatctgtctgtggttaccaaggattctggtgttcctcaaatgtcttccacaggaactgtgcatatcacagttatagaccaaaatgacaatccttcacagtctcggacggtggagatatttgttaattattatggtaacttgtttcccggtgggattttaggctctgtgaagccacaggatccagatgtgttagacagcttccactgctcccttacttcaggagttaccagcctcttcagtattccagggggtacttgtgatctgaattcccagccaaggtccacagatggcacgtttgatctgactgtccttagcaatgatggagttcacagcacagtcacgagcaacatccgagttttctttgctggattttccaatgccacagtggataacagcatcttacttcgtctcggcgtaccaacagtaaaggacttcttgaccaaccactatcttcattttttacgcattgccagctcacagctgacaggcttagggactgctgtgcaactgtacagtgcatatgaagagaacaatagaacgtttcttttggcagctgtgaagcgaaatcataatcagtatgtgaatcccagtggcgtagccaccttctttgaaagcatcaaagagatccttctccggcagagtggagtaaaggtggaatctgtggatcatgactcctgtgtgcatggcccatgtcagaatggagggagctgtctacgaagattggctgtgagctccgtattaaaaagccgtgagagtcttccagtcatcatcgtggcaaatgaacctctgcagcctttcttatgcaagtgtctgccaggatatgcgggtagctggtgtgaaatagatatagatgaatgtcttccatcaccttgccacagtggtggaacctgtcacaatttagtgggaggattttcatgcagctgcccagatggcttcactggtagggcgtgtgagagagatatcaatgagtgcctgcagagtccttgcaagaatggtgccatctgccagaattttccaggaagcttcaactgtgtttgcaaaactggatacacagggaaaatgtgtgaatcttcagtcaattactgtgaatgcaacccctgctttaatggtggttcctgccaaagtggtgtggattcttattattgtcattgtccatttggtgtctttggaaaacactgcgagttgaacagttatggatttgaggagttatcatacatggaatttccaagcttggaccccaataacaactatatttatgtcaaatttgccacgattaaaagtcatgccttattgctttacaactatgacaaccagacaggcgaccgggctgagtttttggcccttgaaattgccgaagaaagactaagattctcttataatttaggcagtggtacatataagctcaccaccatgaagaaggtgtcagatggacattttcacactgtgattgccaggagagcaggaatggcagcctccttaactgtggactcctgttctgagaaccaagagccaggatattgtactgtcagtaatgtggcagtttcagatgactggactcttgatgttcagccaaatagagttacagttggaggtatcagatctctagaaccaatccttcagagaagaggacacgtggaaagccatgattttgttgggtgtataatggagtttgcagtcaatggaaggcctctggaacccagccaagctttggcagcacaaggcatcctagatcaatgccctaggctggaaggcgcttgtactcgcagcccatgccaacatggtggcacatgtatggattactggtcatggcagcagtgtcattgcaaagagggactcactgggaaatactgtgaaaaatctgttactcctgacactgccttatcattagaaggcaaagggcgcttggactaccacatgagtcagaatgagaagcgggaatatttgttaaggcaaagcttacgaggtgccatgttggagccttttggtgtgaacagtctggaagtaaaatttaggaccagaagcgagaatggcgttttaatccatatccaagaaagcagcaattacactactgtgaagattaagaatggcaaagtatattttacatccgatgcaggaattgctgggaaagtggagagaaatattcctgaagtatatgttgcagacggccactggcacacttttctaattgggaaaaatggaacagcaacagtattgtctgttgacagaatatataacagagatattatccaccctactcaggacttcggtggccttgatgtgcttactatatcacttggaggaattccacccaatcaagcacatcgagatgcccaaacagcaggttttgatggctgcattgcttctatgtggtatggtggagaaagtcttcctttcagcgggaagcatagcttggcctccatctcaaaaacagatccctcagtgaagattggctgccgtggcccgaacatttgtgccagcaacccctgctggggtgatttgctgtgcattaatcagtggtatgcctacaggtgtgtccctcctggggactgtgcctcccacccgtgccagaatggtggcagctgtgagccaggcctgcactccggcttcacctgtagctgcccagactcgcacacgggaaggacctgtgagatggtggtggcctgtcttggcgtcctctgtcctcaggggaaggtgtgcaaagctggaagtcctgcggggcatgtctgtgttctgagtcagggccctgaagagatctctctgcctttgtgggctgtgcctgccatcgtgggcagctgcgcaaccgtcttggccctcctggtccttagcctgatcctgtgtaaccagtgcagggggaagaaggccaaaaatcccaaagaggagaagaaaccgaaggagaagaagaaaaagggaagtgagaacgttgcttttgatgaccctgacaatatccctccctatggggatgacatgactgtgaggaagcagcctgaagggaacccaaaaccagatatcattgaaagggaaaacccctaccttatctatgatgaaactgatattcctcacaactcagaaaccatccccagcgcccctttggcatctccagagcaggagatagagcactatgacattgacaacgccagcagcatcgccccttcggatgcagacatcattcaacactacaagcagttccgcagccacacaccaaaattttcaatccagaggcacagtcccctaggctttgcaaggcaatcccccatgcccttaggagcaagcagtttgacttaccagccttcatatggtcaaggtttgagaaccagctccctaagccactcagcatgcccaactcccaaccctctgtctcgacacagtccagcccctttctccaaatcttctacgttctatagaaacagcccagcaagggaattgcatcttcctataagggatggtaatactttggaaatgcatggtgacacctgccaacctggcattttcaactatgccacaaggctgggaaggagaagcaagagtcctcaggccatggcatcacatggttctagaccagggagtcgcctaaagcagccgattgggcagattccactggaatcttctcctccagtcggactttctattgaagaagtggagaggctcaacacacctcgccctagaaacccaagtatctgcagtgcagaccatgggaggtcttcttcagaggaggactgcagaaggccactgtctagaacaaggaatccagcggatggcattccagctccagaatcctcttctgatagtgactcccatgaatctttcacttgctcagaaatggaatatgacagggagaagccaatggtatatacttccagaatgcccaaattatctcaagtcaatgaatctgatgcagatgatgaagataattatggagccagactgaagcctcgaaggtaccacggtcgcagggccgagggaggacctgtgggcacccaggcagcagcaccaggcactgctgacaacacactgcccatgaagctagggcagcaagcagggactttcaactgggacaaccttttgaactggggccctggctttggccattatgtagatgtttttaaagatttggcatctcttccagaaaaagcagcagcaaatgaagaaggcaaagctgggacaactaaaccagtccccaaagatggggaagcagaacagtatgtgtgaagtttatgtactggcactataaaatataaaaacaagaaataatactcaaaccattgtaaagttgctgactaggttgggtcacatttgaaaaacaggccagtatggactagtggtggagggaaaactttaaaaataataaccacaatgctgctgaaacagactcacaacaactcttaatttaaacatgtgtggttgaatttatttccctgcatgcattgtgttttgtaactagttatgtggcatgcagcatttggaaaatttttcttatttaccagtgtttgatttgtgatttttaaaaattgatacctttaccattgcagaaaagaacttgtgctttcccagtggcgtatgtgtattgtttcaactgtattattataattatattttgcattgcaagattcttgatgttaaaccaatccttgtaaagtgtaaaaaggaaccctcctatcgtggaatgaaagattaagtatattaacacttttcagaatgatagtttctgtatttgatgttgctcagaaatgtctgagtatttgagtaagttttacatgacagtgggtactgaaattaagtcattttgttcagcactttaacgctttcttatagaattgtcttaaaacttctggatccttgagcaaatgattatagtctcctgactttcatgaggcttccattaggaacagaatgattgcatgttgtccccagaacactgccaccttgctatgcgaatgatgttctcagcagcacttctaagaaacactcttaaaagttatttattgaaaatttttcgtatgcttttaatattttaaagaattgacctaaggaaagcttatgattggacttattttccaaccagataacatttactctaagtacccagtttttataatttatatgaaatcagatttcaacacttactttgtcattttgtagatcattttttttaaatactgtgtaaaaactttttttacacctaagctgtgtttttgatactgatattttcctatgctgaatagttttcttactttcagggaaggtaagaaaatactttttttatatttgttacttatgtaacattcatatttttctcattttgatatttgtaacatactgtatgctttctacttgtaaatgtcaacaatagaattaaaatatttatttaaaataaaaa

SEQ ID NO: 2 - protein, 4983 aa, Homo sapiens
MDLAPDRATGRPWLPLHTLSVSQLLRVFWLLSLLPGQAWVHGAEPRQVFQVLEEQPPGTLVGTIQTRPGFTYRLSESHALFAINSSTGALYTTSTIDRESLPSDVINLVVLSSAPTYPTEVRVLVRDLNDNAPVFPDPSIVVTFKEDSSSGRQVILDTATDSDIGSNGVDHRSYRIIRGNEAGRFRLDITLNPSGEGAFLHLVSKGGLDREVTPQYQLLVEVEDKGEPKRRGYLQVNVTVQDINDNPPVFGSSHYQAGVPEDAVVGSSVLQVAAADADEGTNADIRYRLQDEGTPFQMDPETGLITVREPLDFEARRQYSLTVQAMDRGVPSLTGRAEALIQLLDVNDNDPVVKFRYFPATSRYASVDENAQVGTVVALLTVTDADSPAANGNISVQILGGNEQRHFEVQSSKVPNLSLIKVASALDRERIPSYNLTVSVSDNYGAPPGAAVQARSSVASLVIFVNDINDHPPVFSQQVYRVNLSEEAPPGSYVSGISATDGDSGLNANLRYSIVSGNGLGWFHISEHSGLVTTGSSGGLDRELASQIVLNISARDQGVHPKVSYAQLVVTLLDVNDEKPVFSQPEGYDVSVVENAPTGTELLMLRATDGDLGDNGTVRFSLQEAETDRRSFRLDPVSGRLSTISSLDREEQAFYSLLVLATDLGSPPQSSMARINVSLLDINDNSPVFYPVQYFAHIKENEPGGSYITTVSATDPDLGTNGTVKYSISAGDRSRFQVNAQSGVISTRMALDREEKTAYQLQIVATDGGNLQSPNQAIVTITVLDTQDNPPVFSQVAYSFVVFENVALGYHVGSVSASTMDLNSNISYLITTGDQKGMFAINQVTGQLTTANVIDREEQSFYQLKVVASGGTVTGDTMVNITVKDLNDNSPHFLQAIESVNVVENWQAGHSIFQAKAVDPDEGVNGMVLYSLKQNPKNLFAINEKNGTISLLGPLDVHAGSYQIEILASDMGVPQLSSSVILTVYVHDVNDNSPVFDQLSYEVTLSESEPVNSRFFKVQASDKDSGANGEIAYTIAEGNTGDAFGIFPDGQLYIKSELDRELQDRYVLMVVASDRAVEPLSATVNVTVILEDVNDNRPLFNSTNYTFYFEEEQRAGSFVGKVSAVDKDFGPNGEVRYSFEMVQPDFELHAISGEITNTHQFDRESLMRRRGTAVFSFTVIATDQGIPQPLKDQATVHVYMKDINDNAPKFLKDFYQATISESAANLTQVLRVSASDVDEGNNGLIHYSIIKGNEERQFAIDSTSGQVTLIGKLDYEATPAYSLVIQAVDSGTIPLNSTCTLNIDILDENDNTPSFPKSTLFVDVLENMRIGELVSSVTATDSDSGDNADLYYSITGTNNHGTFSISPNTGSIFLAKKLDFETQSLYKLNITAKDQGRPPRSSTMSVVIHVRDFNDNPPSFPPGDIFKSIVENIPIGTSVISVTAHDPDADINGQLSYTIIQQMPRGNHFTIDEVKGTIYTNAEIDREFANLFELTVKANDQAVPIETRRYALKNVTILVTDLNDNVPMFISQNALAADPSAVIGSVLTTIMAADPDEGANGEIEYEIINGDTDTFIVDRYSGDLRVASALVPSQLIYNLIVSATDLGPERRKSTTELTIILQGLDGPVFTQPKYITILKEGEPIGTNVISIEAASPRGSEAPVEYYIVSVRCEEKTVGRLFTIGRHTGIIQTAAILDREQGACLYLVDVYAIEKSTAFPRTQRAEVEITLQDINDNPPVFPTDMLDLTVEENIGDGSKIMQLTAMDADEGANALVTYTIISGADDSFRIDPESGDLIATRRLDRERRSKYSLLVRADDGLQSSDMRINITVSDVNDHTPKFSRPVYSFDIPEDTIPGSLVAAILATDDDSGVNGEITYIVNEDDEDGIFFLNPITGVFNLTRLLDYEVQQYYILTVRAEDGGGQFTTIRVYFNILDVNDNPPIFSLNSYSTSLMENLPVGSTVLVFNVTDADDGINSQLTYSIASGDSLGQFTVDKNGVLKVLKALDRESQSFYNLVVQVHDLPQIPASRFTSTAQVSIILLDVNDNPPTFLSPKLTYIPENTPIDTVVFKAQATDPDSGPNSYIEYTLLNPLGNKFSIGTIDGEVRLTGELDREEVSNYTLTVVATDKGQPSLSSSTEVVVMVLDINDNNPIFAQALYKVEINENTLTGTDIIQVFAADGDEGTNGQVRYGIVNGNTNQEFRIDSVTGAITVAKPLDREKTPTYHLTVQATDRGSTPRTDTSTVSIVLLDINDFVPVFELSPYSVNVPENLGTLPRTILQVVARDDDRGSNSKLSYVLFGGNEDNAFTLSASGELGVTQSLDRETKERFVLMITATDSGSPALTGTGTINVIVDDVNDNVPTFASKAYFTTIPEDAPTGTDVLLVNASDADASKNAVISYRIIGGNSQFTINPSTGQIITSALLDRETKDNYTLVVVCSDAGSPEPLSSSTSVLVTVTDVNDNPPRFQHHPYVTHIPSPTLPGSFVFAVTVTDADIGPNSELHYSLSGRNSEKFHIDPLRGAIMAAGPLNGASEVTFSVHVKDGGSFPKTDSTTVTVRFVNKADFPKVRAKEQTFMFPENQPVSSLVTTITGSSLRGEPMSYYIASGNLGNTFQIDQLTGQVSISQPLDFEKIQKYVVWIEARDGGFPPFSSYEKLDITVLDVNDNAPIFKEDPFISEILENLSPRKILTVSAMDKDSGPNGQLDYEIVNGNMENSFSINHATGEIRSVRPLDREKVSHYVLTIKSSDKGSPSQSTSVKVMINILDENDNAPRFSQIFSAHVPENSPLGYTVTRVTTSDEDIGINAISRYSIMDASLPFTINPSTGDIVISRPLNREDTDRYRIRVSAHDSGWTVSTDVTIFVTDINDNAPRFSRTSYYLDCPELTEIGSKVTQVFATDPDEGSNGQVFYFIKSQSEYFRINATTGEIFNKQILKYQNVTGFSNVNINRHSFIVTSSDRGKPSLISETTVTINIVDSNDNAPQFLKSKYFTPVTKNVKVGTKLIRVTAIDDKDFGLNSEVEYFISNDNHLGKFKLDNDTGWISVASSLISDLNQNFFITVTAKDKGNPPLSSQATVHITVTEENYHTPEFSQSHMSATIPESHSIGSIVRTVSARDRDAAMNGLIKYSISSGNEEGIFAINSSTGILTLAKALDYELCQKHEMTISAIDGGWVARTGYCSVTVNVIDVNDNSPVFLSDDYFPTVLENAPSGTTVIHLNATDADSGTNAVIAYTVQSSDSDLFVIDPNTGVITTQGFLDFETKQSYHLTVKAFNVPDEERCSFATVNIQLKGTNEYVPRFVSKLYYFEISEAAPKGTIVGEVFASDRDLGTDGEVHYLIFGNSRKKGFQINKKTGQIYVSGILDREKEERVSLKVLAKNFGSIRGADIDEVTVNVTVLDANDPPIFTLNIYSVQISEGVPIGTHVTFVSAFDSDSIPSWSRFSYFIGSGNENGAFSINPQTGQITVTAELDRETLPIYNLSVLAVDSGTPSATGSASLLVTLEDINDNGPMLTVSEGEVMENKRPGTLVMTLQSTDPDLPPNQGPFTYYLLSTGPATSYFSLSTAGVLSTTREIDREQIADFYLSVVTKDSGVPQMSSTGTVHITVIDQNDNPSQSRTVEIFVNYYGNLFPGGILGSVKPQDPDVLDSFHCSLTSGVTSLFSIPGGTCDLNSQPRSTDGTFDLTVLSNDGVHSTVTSNIRVFFAGFSNATVDNSILLRLGVPTVKDFLTNHYLHFLRIASSQLTGLGTAVQLYSAYEENNRTFLLAAVKRNHNQYVNPSGVATFFESIKEILLRQSGVKVESVDHDSCVHGPCQNGGSCLRRLAVSSVLKSRESLPVIIVANEPLQPFLCKCLPGYAGSWCEIDIDECLPSPCHSGGTCHNLVGGFSCSCPDGFTGRACERDINECLQSPCKNGAICQNFPGSFNCVCKTGYTGKMCESSVNYCECNPCFNGGSCQSGVDSYYCHCPFGVFGKHCELNSYGFEELSYMEFPSLDPNNNYIYVKFATIKSHALLLYNYDNQTGDRAEFLALEIAEERLRFSYNLGSGTYKLTTMKKVSDGHFHTVIARRAGMAASLTVDSCSENQEPGYCTVSNVAVSDDWTLDVQPNRVTVGGIRSLEPILQRRGHVESHDFVGCIMEFAVNGRPLEPSQALAAQGILDQCPRLEGACTRSPCQHGGTCMDYWSWQQCHCKEGLTGKYCEKSVTPDTALSLEGKGRLDYHMSQNEKREYLLRQSLRGAMLEPFGVNSLEVKFRTRSENGVLIHIQESSNYTTVKIKNGKVYFTSDAGIAGKVERNIPEVYVADGHWHTFLIGKNGTATVLSVDRIYNRDIIHPTQDFGGLDVLTISLGGIPPNQAHRDAQTAGFDGCIASMWYGGESLPFSGKHSLASISKTDPSVKIGCRGPNICASNPCWGDLLCINQWYAYRCVPPGDCASHPCQNGGSCEPGLHSGFTCSCPDSHTGRTCEMVVACLGVLCPQGKVCKAGSPAGHVCVLSQGPEEISLPLWAVPAIVGSCATVLALLVLSLILCNQCRGKKAKNPKEEKKPKEKKKKGSENVAFDDPDNIPPYGDDMTVRKQPEGNPKPDIIERENPYLIYDETDIPHNSETIPSAPLASPEQEIEHYDIDNASSIAPSDADIIQHYKQFRSHTPKFSIQRHSPLGFARQSPMPLGASSLTYQPSYGQGLRTSSLSHSACPTPNPLSRHSPAPFSKSSTFYRNSPARELHLPIRDGNTLEMHGDTCQPGIFNYATRLGRRSKSPQAMASHGSRPGSRLKQPIGQIPLESSPPVGLSIEEVERLNTPRPRNPSICSADHGRSSSEEDCRRPLSRTRNPADGIPAPESSSDSDSHESFTCSEMEYDREKPMVYTSRMPKLSQVNESDADDEDNYGARLKPRRYHGRRAEGGPVGTQAAAPGTADNTLPMKLGQQAGTFNWDNLLNWGPGFGHYVDVFKDLASLPEKAAANEEGKAGTTKPVPKDGEAEQYV

SEQ ID NO: 3 - DNA, 3286 bp, Homo sapiens
gcgtgtcgggcgcggaagggggaggcggcccggggcgcccgcgagtgaggcgcggggcggcgaagggagcgcgggtggcggcacttgctgccgcggccttggatgggctgggcccccctcgccgctccgcctcctccacacgcgcggcggccgcggcgagggggacgcgccgcccggggcccggcaccttcgggaaccccccggcccggagcctgcggcctgcgccgcctcggccgccgggagccccgtggagcccccgccgccgcgccgccccgcggaccggacgctgagggcactcggggcggggcgcgcgctcgggcagacgtttgcggggaggggggcgcctgccgggccccggcgaccaccttgggggtcgcgggccggctcggggggcgcccagtgcgggccctcgcgggcgccgggcagcgaccagccctgagcggagctgttggccgcggcgggaggcctcccggacgcccccagccccccgaacgctcgcccgggccggcgggagtcggcgccccccgggaggtccgctcggtcgtccgcggcggagcgtttgctcctgggacaggcggtgggaccggggcgtcgccggagacgcccccagcgaagttgggctctccaggtgtgggggtcccggggggtagcgacgtcgcggacccggcctgtgggatgggcggcccggagaagactgcgctcggccgtgttcatacttgtccgtgggcctgaggtccccggaggatgacctagcactgaaaagccccggccggcctccccagggtccccgaggacgaagttgaccctgaccgggccgtctcccagttctgaggcccgggtcccactggaactcgcgtctgagccgccgtcccggacccccggtgcccgccggtccgcagaccctgcaccgggcttggactcgcagccgggactgacgtgtagaacaatcgtttctgttggaagaagggtttttcccttccttttggggtttttgttgccttttttttttcttttttctttgtaaaattttggagaagggaagtcggaacacaaggaaggaccgctcacccgcggactcagggctggcggcgggactccaggaccctgggtccagcatggaggtggtggacccgcagcagctgggcatgttcacggagggcgagctgatgtcggtgggtatggacacgttcatccaccgcatcgactccaccgaggtcatctaccagccgcgccgcaagcgggccaagctcatcggcaagtacctgatgggggacctgctgggggaaggctcttacggcaaggtgaaggaggtgctggactcggagacgctgtgcaggagggccgtcaagatcctcaagaagaagaagttgcgaaggatccccaacggggaggccaacgtgaagaaggaaattcaactactgaggaggttacggcacaaaaatgtcatccagctggtggatgtgttatacaacgaagagaagcagaaaatgtatatggtgatggagtactgcgtgtgtggcatgcaggaaatgctggacagcgtgccggagaagcgtttcccagtgtgccaggcccacgggtacttctgtcagctgattgacggcctggagtacctgcatagccagggcattgtgcacaaggacatcaagccggggaacctgctgctcaccaccggtggcaccctcaaaatctccgacctgggcgtggccgaggcactgcacccgttcgcggcggacgacacctgccggaccagccagggctccccggctttccagccgcccgagattgccaacggcctggacaccttctccggcttcaaggtggacatctggtcggctggggtcaccctctacaacatcaccacgggtctgtaccccttcgaaggggacaacatctacaagttgtttgagaacatcgggaaggggagctacgccatcccgggcgactgtggccccccgctctctgacctgctgaaagggatgcttgagtacgaaccggccaagaggttctccatccggcagatccggcagcacagctggttccggaagaaacatcctccggctgaagcaccagtgcccatcccaccgagcccagacaccaaggaccggtggcgcagcatgactgtggtgccgtacttggaggacctgcacggcgcggacgaggacgaggacctcttcgacatcgaggatgacatcatctacactcaggacttcacggtgcccggacaggtcccagaagaggaggccagtcacaatggacagcgccggggcctccccaaggccgtgtgtatgaacggcacagaggcggcgcagctgagcaccaaatccagggcggagggccgggcccccaaccctgcccgcaaggcctgctccgccagcagcaagatccgccggctgtcggcctgcaagcagcagtgaggctggccgcctgcagcccgtgtccaggagccccgccaggtgcccgcgccaggccctcagtcttcctgccggttccgcccgccctcccggagaggtggccgccatgcttctgtgccgaccacgccccaggacctccggagcgccctgcagggccgggcagggggacagcagggaccgggcgcagccctcccccctcggccgcccggcagtgcacgcggcttgttgacttcgcagccccgggcggagccttcccgggcgggcgtgggaggagggaggcggcctccatgcactttatgtggagactactggccccgcccgtggcctcgtgctccgcagggcgcccagcgccgtccggcggccccgccgcagaccagctggcgggtgtggagaccaggctcctgaccccgccatgcatgcagcgccacctggaagccgcgcggccgctttggttttttgtttggttggttccattttctttttttctttttttttttaagaaaaaataaaaggtggatttgagctgtggctgtgaggggtgtttgggagctgctgggtggcaggggggctgtggggtcgggctcacgtcgcggccgcctttgcgctctcgggtcaccctgctttggcggcccggccggagggcaggaccctcacctctcccccaaggccactgcgctcttgggaccccagagaaaacccggagcaagcaggagtgtgcggtcaatatttatatcatccagaaaagaaaaacacgagaaacgccatcgcgggatggtgcagacgcggcggggactcggagggtgccgtgcgggcgaggccgcccaaatttggcaataaataaagcttgggaagcttggacctgaaaaaaaaaa

SEQ ID NO: 4 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

SEQ ID NO: 5 - DNA, 7533 bp, Homo sapiens
gcgacgctcacgaacgatcagagctgcgggcgacgcaacgaagcccggaggccgcaggctgcgcgctccctcgcagcagccgggcgggcaaaagcccccagtcctcggcccccgcgcaagcgacgccgggaaatgcccacatccgggaaacctgcagcggagtgcggcggcggcgacactgagtggaaggcaaaatggcggcggcggcggcggtggcctggtgttaaggggagagccaggtcctcacgacccctgggacgggccgcgctggcccgcggcagcccccccgttcgtctccccgctctgccccaccagggatacttggggttgctgggacggactctggccgcctcagcgtccgccctcaggcccgtggccgctgtccaggagctctgctctcccctccagagttaattatttatattgtaaagaattttaacagtcctggggacttccttgaaggatcattttcacttttgctcagaagaaagctctggatctatcaaataaagaagtccttcgtgtgggctacatatatagatgttttcatgaagaggagtgaaaagccagaaggatatagacaaatgaggcctaagacctttcctgccagtaactatactgtcagtagccggcaaatgttacaagaaattcgggaatcccttaggaatttatctaaaccatctgatgctgctaaggctgagcataacatgagtaaaatgtcaaccgaagatcctcgacaagtcagaaatccacccaaatttgggacgcatcataaagccttgcaggaaattcgaaactctctgcttccatttgcaaatgaaacaaattcttctcggagtacttcagaagttaatccacaaatgcttcaagacttgcaagctgctggatttgatgaggatatggttatacaagctcttcagaaaactaacaacagaagtatagaagcagcaattgaattcattagtaaaatgagttaccaagatcctcgacgagagcagatggctgcagcagctgccagacctattaatgccagcatgaaaccagggaatgtgcagcaatcagttaaccgcaaacagagctggaaaggttctaaagaatccttagttcctcagaggcatggcccgccactaggagaaagtgtggcctatcattctgagagtcccaactcacagacagatgtaggaagacctttgtctggatctggtatatcagcatttgttcaagctcaccctagcaacggacagagagtgaaccccccaccaccacctcaagtaaggagtgttactcctccaccacctccaagaggccagactccccctccaagaggtacaactccacctcccccttcatgggaaccaaactctcaaacaaagcgctattctggaaacatggaatacgtaatctcccgaatctctcctgtcccacctggggcatggcaagagggctatcctccaccacctctcaacacttcccccatgaatcctcctaatcaaggacagagaggcattagttctgttcctgttggcagacaaccaatcatcatgcagagttctagcaaatttaactttccatcagggagacctggaatgcagaatggtactggacaaactgatttcatgatacaccaaaatgttgtccctgctggcactgtgaatcggcagccaccacctccatatcctctgacagcagctaatggacaaagcccttctgctttacaaacagggggatctgctgctccttcgtcatatacaaatggaagtattcctcagtctatgatggtgccaaacagaaatagtcataacatggaactatataacattagtgtacctggactgcaaacaaattggcctcagtcatcttctgctccagcccagtcatccccgagcagtgggcatgaaatccctacatggcaacctaacataccagtgaggtcaaattcttttaataacccattaggaaatagagcaagtcactctgctaattctcagccttctgctacaacagtcactgcaattacaccagctcctattcaacagcctgtgaaaagtatgcgtgtattaaaaccagagctacagactgctttagcacctacacacccttcttggataccacagccaattcaaactgttcaacccagtccttttcctgagggaaccgcttcaaatgtgactgtgatgccacctgttgctgaagctccaaactatcaaggaccaccaccaccctacccaaaacatctgctgcaccaaaacccatctgttcctccatacgagtcaatcagtaagcctagcaaagaggatcagccaagcttgcccaaggaagatgagagtgaaaagagttatgaaaatgttgatagtggggataaagaaaagaaacagattacaacttcacctattactgttaggaaaaacaagaaagatgaagagcgaagggaatctcgtattcaaagttattctcctcaagcatttaaattctttatggagcaacatgtagaaaatgtactcaaatctcatcagcagcgtctacatcgtaaaaaacaattagagaatgaaatgatgcgggttggattatctcaagatgcccaggatcaaatgagaaagatgctttgccaaaaagaatctaattacatccgtcttaaaagggctaaaatggacaagtctatgtttgtgaagataaagacactaggaataggagcatttggtgaagtctgtctagcaagaaaagtagatactaaggctttgtatgcaacaaaaactcttcgaaagaaagatgttcttcttcgaaatcaagtcgctcatgttaaggctgagagagatatcctggctgaagctgacaatgaatgggtagttcgtctatattattcattccaagataaggacaatttatactttgtaatggactacattcctgggggtgatatgatgagcctattaattagaatgggcatctttccagaaagtctggcacgattctacatagcagaacttacctgtgcagttgaaagtgttcataaaatgggttttattcatagagatattaaacctgataatattttgattgatcgtgatggtcatattaaattgactgactttggcctctgcactggcttcagatggacacacgattctaagtactatcagagtggtgaccatccacggcaagatagcatggatttcagtaatgaatggggggatccctcaagctgtcgatgtggagacagactgaagccattagagcggagagctgcacgccagcaccagcgatgtctagcacattctttggttgggactcccaattatattgcacctgaagtgttgctacgaacaggatacacacagttgtgtgattggtggagtgttggtgttattctttttgaaatgttggtgggacaacctcctttcttggcacaaacaccattagaaacacaaatgaaggttatcaactggcaaacatctcttcacattccaccacaagctaaactcagtcctgaagcttctgatcttattattaaactttgccgaggacccgaagatcgcttaggcaagaatggtgctgatgaaataaaagctcatccattttttaaaacaattgacttctccagtgacctgagacagcagtctgcttcatacattcctaaaatcacacacccaacagatacatcaaattttgatcctgttgatcctgataaattatggagtgatgataacgaggaagaaaatgtaaatgacactctcaatggatggtataaaaatggaaagcatcctgaacatgcattctatgaatttaccttccgaaggttttttgatgacaatggctacccatataattatccgaagcctattgaatatgaatacattaattcacaaggctcagagcagcagtcggatgaagatgatcaaaacacaggctcagagattaaaaatcgcgatctagtatatgtttaacacactagtaaataaatgtaatgaggatttgtaaaagggcctgaaatgcgaggtgttttgaggttctgagagtaaaattatgcaaatatgacagagctatatatgtgtgctctgtgtacaatattttattttcctaaattatgggaaatccttttaaaatgttaatttattccagccgtttaaatcagtatttagaaaaaaattgttataaggaaagtaaattatgaactgaatattatagtcagttcttggtacttaaagtacttaaaataagtagtgctttgtttaaaaggagaaacctggtatctatttgtatatatgctaaataattttaaaatacaagagtttttgaaatttttttgaaagacagttttagttttatcttgctttaaccaaatatgaaacataccccctattttacagagctcttttttcccctcataaccttgtttttggtagaaaataagctagagaaattaagccatcgtgttggtgagtgttcctaggctaatgataatctgtataattcacatcctgaaactaaggaatacagggttgaaaaaatattaatatgtttgtcagaaggaaaaataatgcatttatcttcccccccaccccccgccccatggaatatttaatctatttaatcttcttgcatttatttctcaagaattactggctttaaaagaagccaaagcactactagctttttttccatattggtatttttgatgctgcttccaattttaaaagggaacaaagctgccataaatcgaaatgttcaatactaaaagctaaaatatttctcaccatcctaagcagataattattttaattttcatatacttttcctgtatagtaactattttgattatatcatcaatgttacctgtttcctctttcagaacagtgctgcatatacagattgttattggcaaaggaaaatctggctatctggcaatattttacctaagcgcagattaattggtgaaaaaattaactcttaagatggccattaataattaggaaagtttacagagtggtcttagtagaaaattcaagtcctcctaatttatttaaggttcaataatgcgttcaacatgcctgttatgtataacgcttaggttctaaggaagattaaggtttcataccaaaatacatgtagcttatcttttaggaaggggaaaaaggctccattttgaccatagtaaaatttgtgttgtgttttatttccttttcttaagctccactgataagggattgtttttatcaaaagttactatttgtagattggaggcataattttagtgattttcatacttttagctttcttcgcataaaagctaattgaaaccgtatatgtagtaaaattaaaggcagagctgttgcagttgaattggagagttagggcaaagaacacttattagcccacacttcccacctttctacaggtggtcctttcagagctcagcctgaaaacccactactgtgttatcgtgcgtcttttggggttagtggttcttttgagaatctgaaggaagctgtggactcttcctagaaaaaaaaaccacacatacacatacaatgttgcatgcagtttcaagggattttggacatattgaaacctatcacaggctgtaggttatggacctctgtgccatgagaaaattgatacattaaactaagaactttgtttttaacttaccaatcactactcagcacatcttatataagctgataatttgtgatggaaaaggtctgtagcatgtgatataaggtgaccttatgaatgcctctcttgctggtacattaagttgttttaatatatcatttggaggggactgaaatgttaggctcattacaagcttgatacagaaatatttctgaaggatttctaatcagaattgtaaaacaatgtgctatcatgaaatcgcagtcttcacctcatggttcatggaacatttggttagtcccataaaatcctatgcaaaacaaagtagttcaagaatttttaggtgggtagtcacatttataaggtattcctcttactctttgggctttttcagtctgatttatttaaattttcatttagttgttttacttttggactaaggtgcaatacagtagaagataactttgttacatttatgttgtaggaaaactaaggtgctgtctcctcccccttcccttcccacaaaatctgtattccccctattgctgaaatgtaacagacactacaaattttgtattctttttttgttttttgttttgagacagggtctcactctgtcacccaggctggagggcagtggcgcttcacagctcactgcatcctcaaccttgggggctcacgcagtcctcccgcctcagcctcccaagtagctgggcatgcgccaccaagcccagctaatttttgtatctttagtagagatggggtttcgccatgttgcccaggttggtgtggaattcctgggctccagttatatgcccacctcagcctcccaaagtgctgggattacagacgtgacccaccgcgcctggcgcaaatatgtattcttttaaaatttcctctgatactataagctttttgcatttatctgaagcagtatacatgcctttggtatcagcaattttaacagtttggatatacttatcagctatcttattccaaaactacatctacttcttccagtatagaatctggtgcttcctgaccaaaaagatgagaaaaacaatgttaaaaatatagatgctttccattgaaatggagtgaaaacattggttctatatgttttcttttaaaataattttcttattaaaaacttgctgtctttattatacttaccctttttatgcatatcaatagtatttataagatgtgttctataattatgtaattgtagatactgttatgcattgtccagtgacatcataaggcaggccctactgctgtatcttttctaccttcttatttgtaatagaaactatagaatgtatgactaaaaagtcactttgagattgacttttttaaaaagttattaccttctgctgttgcaaagtgcaaaactgtgagtggaattgttttattctgacttaatgtgttagaaattagagaatacagtgggaggatttttagacattgctgctgctgttacccaaggtattttagataaaaaatttttaataaacatccctttggtatttaaagtggaacatttagcctgttcattttaatctaaagcaaaaagtaatttgggtcaaaatattggtatatttgtaaagcgccttaatatatccctttgtggaaggcactacacagtttacttttatattgtattgtgtatataagtattttgtattaaaattgaatcagtggcaacattaaagttttataaaatcatgctttgttagaaaaagaattacagctttgcaatataactaattgtttcgcataattctgaatgtaatagatatgaataatcagcctgtgtttttaatgaacttatttgtattttcccaatcattttctctagtgtaatgtttgctgggataataaaaaaaattcaaatctttcaaaaaaaaaaaaaaaaaa

SEQ ID NO: 6 - protein, 1130 aa, Homo sapiens
MKRSEKPEGYRQMRPKTFPASNYTVSSRQMLQEIRESLRNLSKPSDAAKAEHNMSKMSTEDPRQVRNPPKFGTHHKALQEIRNSLLPFANETNSSRSTSEVNPQMLQDLQAAGFDEDMVIQALQKTNNRSIEAAIEFISKMSYQDPRREQMAAAAARPINASMKPGNVQQSVNRKQSWKGSKESLVPQRHGPPLGESVAYHSESPNSQTDVGRPLSGSGISAFVQAHPSNGQRVNPPPPPQVRSVTPPPPPRGQTPPPRGTTPPPPSWEPNSQTKRYSGNMEYVISRISPVPPGAWQEGYPPPPLNTSPMNPPNQGQRGISSVPVGRQPIIMQSSSKFNFPSGRPGMQNGTGQTDFMIHQNVVPAGTVNRQPPPPYPLTAANGQSPSALQTGGSAAPSSYTNGSIPQSMMVPNRNSHNMELYNISVPGLQTNWPQSSSAPAQSSPSSGHEIPTWQPNIPVRSNSFNNPLGNRASHSANSQPSATTVTAITPAPIQQPVKSMRVLKPELQTALAPTHPSWIPQPIQTVQPSPFPEGTASNVTVMPPVAEAPNYQGPPPPYPKHLLHQNPSVPPYESISKPSKEDQPSLPKEDESEKSYENVDSGDKEKKQITTSPITVRKNKKDEERRESRIQSYSPQAFKFFMEQHVENVLKSHQQRLHRKKQLENEMMRVGLSQDAQDQMRKMLCQKESNYIRLKRAKMDKSMFVKIKTLGIGAFGEVCLARKVDTKALYATKTLRKKDVLLRNQVAHVKAERDILAEADNEWVVRLYYSFQDKDNLYFVMDYIPGGDMMSLLIRMGIFPESLARFYIAELTCAVESVHKMGFIHRDIKPDNILIDRDGHIKLTDFGLCTGFRWTHDSKYYQSGDHPRQDSMDFSNEWGDPSSCRCGDRLKPLERRAARQHQRCLAHSLVGTPNYIAPEVLLRTGYTQLCDWWSVGVILFEMLVGQPPFLAQTPLETQMKVINWQTSLHIPPQAKLSPEASDLIIKLCRGPEDRLGKNGADEIKAHPFFKTIDFSSDLRQQSASYIPKITHPTDTSNFDPVDPDKLWSDDNEEENVNDTLNGWYKNGKHPEHAFYEFTFRRFFDDNGYPYNYPKPIEYEYINSQGSEQQSDEDDQNTGSEIKNRDLVYV

SEQ ID NO: 7 - DNA, 5558 bp, Homo sapiens
gcccgtggaatgccaacaatgtagcgaatgtcccacttgggtctgcgctttggaaccgcggcgtgagcgccccgggaagatggagcagtcgccgtccacgccaccgccgccgcccggggctcccccgtccctgcggggccagcagcagctccagccaccagtgcccggtctcccggcgcgagaggcccgggagccgccggccaggacgcccccgagggtgtagaccgcgcccctggagagagtgataatcttcaaaatgaagactttggaaaattttaggttctctataggaactacaaaaatggaaggaaagaacattttcaaaaggaaattattttgaaagtatgtttacaacaaactgatactattgacagtttttttttttaaataataaaacactttaagaagattgtatttatggtaaaaggaaactggactaacaatgaggccaaagacttttcctgccacgacttattctggaaatagccggcagcgactgcaagagattcgtgaggggttaaaacagccatccaagtcttcggttcaggggctacccgcaggaccaaacagtgacacttccctggatgccaaagtcctggggagcaaagatgccaccaggcagcagcagcagatgagagccaccccaaagttcggaccttatcagaaagccttgagggaaatcagatattccttgttgccttttgctaatgaatcgggcacctctgcagctgcagaagtgaaccggcaaatgctgcaggaactggtgaacgcaggatgcgaccaggagatggctggccgagctctcaagcagactggcagcaggagcatcgaggccgccctggagtacatcagcaagatgggctacctggacccgaggaatgagcagattgtgcgggtcattaagcagacctccccaggaaaggggctcatgccaaccccagtgacgcggaggcccagcttcgaaggaaccggcgattcgtttgcgtcctaccaccagctgagcggtaccccctacgagggcccaagcttcggcgctgacggccccacggcgctggaggagatgccgcggccgtacgtggactaccttttccccggagtcggcccccacgggcccggccaccagcaccagcacccacccaagggctacggtgccagcgtagaggcagcaggggcacacttcccgctgcagggcgcgcactacgggcggccgcacctgctggtgcctggggaacccctgggctacggagtgcagcgcagcccctccttccagagcaagacgccgccggagaccgggggttacgccagcctgcccacgaagggccagggaggaccgccaggcgccggcctcgctttcccaccccctgccgccgggctctacgtgccgcacccacaccacaagcaggccggtcccgcggcccaccagctgcatgtgctgggctcccgcagccaggtgttcgccagcgacagccccccgcagagcctgctcactccctcgcggaacagcctcaacgtggacctgtatgaattgggcagcacctccgtccagcagtggccggctgccaccctggcccgccgggactccctgcagaagccgggcctggaggcgccgccgcgcgcgcacgtggccttccggcctgactgcccagtgcccagcaggaccaactccttcaacagccaccagccgcggcccggtccgcctggcaaggccgagccctccctgcccgcccccaacaccgtgacggctgtcacggccgcgcacatcttgcacccggtgaagagcgtgcgtgtgctgaggccggagccgcagacggctgtggggccctcgcaccccgcctgggtgcccgcgcctgccccggcccccgcccccgcccccgccccggctgcggagggcttggacgccaaggaggagcatgccctggcgctgggcggcgcaggcgccttcccgctggacgtggagtacggaggcccagaccggaggtgcccgcctccgccctacccgaagcacctgctgctgcgcagcaagtcggagcagtacgacctggacagcctgtgcgcaggcatggagcagagcctccgtgcgggccccaacgagcccgagggcggcgacaagagccgcaaaagcgccaagggggacaaaggcggaaaggataaaaagcagattcagacctctcccgttcccgtccgcaaaaacagcagagacgaagagaagagagagtcacgcatcaagagctactcgccatacgcctttaagttcttcatggagcagcacgtggagaatgtcatcaaaacctaccagcagaaggttaaccggaggctgcagctggagcaagaaatggccaaagctggactctgtgaagctgagcaggagcagatgcggaagatcctctaccagaaagagtctaattacaacaggttaaagagggccaagatggacaagtctatgtttgtcaagatcaaaaccctggggatcggtgcctttggagaagtgtgccttgcttgtaaggtggacactcacgccctgtacgccatgaagaccctaaggaaaaaggatgtcctgaaccggaatcaggtggcccacgtcaaggccgagagggacatcctggccgaggcagacaatgagtgggtggtcaaactctactactccttccaagacaaagacagcctgtactttgtgatggactacatccctggtggggacatgatgagcctgctgatccggatggaggtcttccctgagcacctggcccggttctacatcgcagagctgactttggccattgagagtgtccacaagatgggcttcatccaccgagacatcaagcctgataacattttgatagatctggatggtcacattaaactcacagatttcggcctctgcactgggttcaggtggactcacaattccaaatattaccagaaagggagccatgtcagacaggacagcatggagcccagcgacctctgggatgatgtgtctaactgtcggtgtggggacaggctgaagaccctagagcagagggcgcggaagcagcaccagaggtgcctggcacattcactggtggggactccaaactacatcgcacccgaggtgctcctccgcaaagggtacactcaactctgtgactggtggagtgttggagtgattctcttcgagatgctggtggggcagccgccctttttggcacctactcccacagaaacccagctgaaggtgatcaactgggagaacacgctccacattccagcccaggtgaagctgagccctgaggccagggacctcatcaccaagctgtgctgctccgcagaccaccgcctggggcggaatggggccgatgacctgaaggcccaccccttcttcagcgccattgacttctccagtgacatccggaagcagccagccccctacgttcccaccatcagccaccccatggacacctcgaatttcgaccccgtagatgaagaaagcccttggaacgatgccagcgaaggtagcaccaaggcctgggacacactcacctcgcccaataacaagcatcctgagcacgcattttacgaattcaccttccgaaggttctttgatgacaatggctacccctttcgatgcccaaagccttcaggagcagaagcttcacaggctgagagctcagatttagaaagctctgatctggtggatcagactgaaggctgccagcctgtgtacgtgtagatgggggccaggcacccccaccactcgctgcctcccaggtcagggtcccggagccggtgccctcacaggccaatagggaagccgagggctgttttgttttaaattagtccgtcgattacttcacttgaaattctgctcttcaccaagaaaacccaaacaggacacttttgaaaacaggactcagcatcgctttcaataggcttttcaggaccttcactgcattaaaacaatatttttgaaaatttagtacagtttagaaagagcacttattttgtttatatccattttttcttactaaattatagggattaactttgacaaatcatgctgctgttattttctacatttgtattttatccatagcacttattcacatttaggaaaagacataaaaactgaagaacattgatgagaaatctctgtgcaataatgtaaaaaaaaaaaaagataacactctgctcaatgtcacggagaccattttatccacacaatggtttttgttttttattttttcccatgtttcaaaattgtgatataatgatataatgttaaaagctgctttttttggctttttgcatatctagtataataggaagtgtgagcaaggtgatgatgtggctgtgatttccgacgtctggtgtgtggagagtactgcatgagcagagttcttctattataaaattaccatatcttgccattcacagcaggtcctgtgaatacgtttttactgagtgtctttaaatgaggtgttctagacagtgtgctgataatgtattgtgcgggtgacctcttcgctatgattgtatctcttactgttttgttaaagaaatgcagatgtgtaactgagaagtgatttgtgtgtgtgtcttggttgtgattggattctttggggggggggaactgaaacatttgtcatatactgaacttatatacatcaaaagggattaatacagcgatgccaaaaagtttaatcacggacacatgtccgtttctgtagtccgtatgctctttcattcttggtagagctggtatgtggaatgccatacctctgaccctactacttacctttttactgacagactgcccacactgaaagcttcagtgaatgttcttagtcctgttttcttctgttactgtcaggaaactgagtgatctaatggttctctcactttttttttgttcttttagtgtactttgaagtatcaaatcttaacttggtttaaacaatacatattcctaacctttgtaaaaaagcaaagattcttcaaaatgacattgaaataaaaagtaagccatacgtattttcttagaagtatagatgtatgtgcgtgtatacacacacacacacacacacagagataaacacaatattccttatttcaaattagtatgattcctatttaaagtgatttatatttgagtaaaaagttcaattcttttttgctttttaaaaaatctgatgcttcataattttcattatattattccacatatttttccttgaagttcttagcataatgtatccattacttagtatatatctaggcaacaacacttagaagtttatcagtgtttaaactaaaaaaataaagattcctgtgtactggtttacatttgtgtgagtggcatactcaagtctgctgtgcctgtcgtcgtgactgtcagtattctcgctattttatagtcgtgccatgttgttactcacagcgctctgacatactttcatgtggtaggttctttctcaggaactcagtttaactattatttattgatatatcattacctttgaaaagcttctactggcacaatttattattaaaattttgaatccaaaaaaaaaaaaaaaaaa

SEQ ID NO: 8 - protein, 1088 aa, Homo sapiens
MRPKTFPATTYSGNSRQRLQEIREGLKQPSKSSVQGLPAGPNSDTSLDAKVLGSKDATRQQQQMRATPKFGPYQKALREIRYSLLPFANESGTSAAAEVNRQMLQELVNAGCDQEMAGRALKQTGSRSIEAALEYISKMGYLDPRNEQIVRVIKQTSPGKGLMPTPVTRRPSFEGTGDSFASYHQLSGTPYEGPSFGADGPTALEEMPRPYVDYLFPGVGPHGPGHQHQHPPKGYGASVEAAGAHFPLQGAHYGRPHLLVPGEPLGYGVQRSPSFQSKTPPETGGYASLPTKGQGGPPGAGLAFPPPAAGLYVPHPHHKQAGPAAHQLHVLGSRSQVFASDSPPQSLLTPSRNSLNVDLYELGSTSVQQWPAATLARRDSLQKPGLEAPPRAHVAFRPDCPVPSRTNSFNSHQPRPGPPGKAEPSLPAPNTVTAVTAAHILHPVKSVRVLRPEPQTAVGPSHPAWVPAPAPAPAPAPAPAAEGLDAKEEHALALGGAGAFPLDVEYGGPDRRCPPPPYPKHLLLRSKSEQYDLDSLCAGMEQSLRAGPNEPEGGDKSRKSAKGDKGGKDKKQIQTSPVPVRKNSRDEEKRESRIKSYSPYAFKFFMEQHVENVIKTYQQKVNRRLQLEQEMAKAGLCEAEQEQMRKILYQKESNYNRLKRAKMDKSMFVKIKTLGIGAFGEVCLACKVDTHALYAMKTLRKKDVLNRNQVAHVKAERDILAEADNEWVVKLYYSFQDKDSLYFVMDYIPGGDMMSLLIRMEVFPEHLARFYIAELTLAIESVHKMGFIHRDIKPDNILIDLDGHIKLTDFGLCTGFRWTHNSKYYQKGSHVRQDSMEPSDLWDDVSNCRCGDRLKTLEQRARKQHQRCLAHSLVGTPNYIAPEVLLRKGYTQLCDWWSVGVILFEMLVGQPPFLAPTPTETQLKVINWENTLHIPAQVKLSPEARDLITKLCCSADHRLGRNGADDLKAHPFFSAIDFSSDIRKQPAPYVPTISHPMDTSNFDPVDEESPWNDASEGSTKAWDTLTSPNNKHPEHAFYEFTFRRFFDDNGYPFRCPKPSGAEASQAESSDLESSDLVDQTEGCQPVYV

SEQ ID NO: 9 - DNA, 6046 bp, Homo sapiens
tgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcagccgtcagggcccgtcccccaactcccctttccgctcaggcagggtcctcgcggcccatgctggccgctggggacccgcgcagcccagaccgttcccgggccgggcagccggccaccatggtggccctgaggcctgtgcagcaactccaggggggctaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggctttccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctaccgggaccgcgggatggaccagatatcaagagagccatccatagggagctggctgggggtttccgtgggagctccagaactttccccagctgagtgaagagcccagcccctcttatgtgcaattgccttgaactacgaccctgtagagatttctctcatggcgttctagttctctgacctgagtctttgttttaagaagtatttgtcttcctttgtctaatgtgggattcctgactcccttcgtccaaggcaccggtgtgtgtgtgtcttgcactccagagctgacctccaccgcccagcctgggaagtcattgtagggagtgagacactgaagccctgagaagccagtgccatcatccccaccccgcccagggttccggaacattcattcccccaccggtgaggacctggcatgcagcgaagcagcccagcccggcggatcccaggccagcacgcctgccggcttctcatcgtcagggagcccgcccagagctcgtgacgagcaagtgctgggtccccgccaggcaccccgaggcggcgctctggctggcagctggtggggaataggcagggcagctgtggctggggagagactttaggcagaagctgtgatgcaggctgactgccagccgaggggctgggtagtgccgtgcgggagctgatggtacagggcactcgctgtccccctccggccaccctagaccagggtccgagaggcaggcaggagccactcatgtcttccccattgcccgacgcccatagacgctccttcctgtgtggggctggggtactccctggtcgtactgcagtcagcacccgtaacccggctatgaccagggatctgtaagccctgtggctccacaggtgctgcttctcactggcccagactctgagctccaccggcccagtctgcacggcccatctgcttcaccttccctcccagccacgtgccagtggccacagcccacttcccaacccactgttgtacccaggcctcactttgctgttgcccttgtccctcttcgggccctgaattttctgttccctgggggccagccagggccctttgtgcccctcccagcacaggcctgatgcaggtgtccactcacaggtggcgctcacctaggctgtcacaggacccacctccatgccaggcaacagagggccacagaaccacccccacggctcactccttggtctggggccaccttcttgccctttctttttttttttttttttttttttccgagatggagtctctctctgtcacccaggtgggagtgtggtggcacaatctcggctcactgcaacctccacctcctgagttcaagcaattctcctgcctcagcctcccaagtagctgggactacaggcatgcaccaccacacttggccaatgttctgtatttttaatagggacggggttttgccatgttagctaggctggtctcaaactcacacctgggattacaggcatgagccactgcacccagccccttcttgccctttcttttctccatggctgatgctgctgtggccagccagggcccttgagatccttccagtttggctgttatgcaaagcaggtgatttgtcttaatcagataaaagatagaggctatgggggcctcaagatttttggagagcagaggtggtctctggcaattccatctggttttgagaaacttagcagctcacagagcacagagatcctgccttcttcctactatcaggctgacctaatggggttgggctgctcggcaactgcttgggtcaccttgccccaaggaaaccagccctgggtgccacccagccacttagggtctacagggtgggactccagacctagagcgtaagtatggatgttgtggccctgtgtcttcctagtgtgacccagccaggagcggaagcttcaggcgtttgtaaagtgaggtctggctctgcctcctccgttttttttttttttctgtttctgtttctgttttttttttgagatggagtctcgctctgtcgcccaggctggagtgcagtgtcacgatctcagctcactgcaacctccgcctcccaggtacaagagattctcctgcctcagcctcccgagtagctgggactacaggcgtgtgccaccatgcctggctaatttttgtatttttagtagagatggggtttcgccatgttagccagactggtctcgaactcctgacctcaggtgatcctcccaccccggcttcccaaagttctgggattataggcgtgagccaccatgcccggtctcttctcagtcttgaagcccatccctggatttccaccaggagttactttcctcctgacctgtaaatttgttctttaacaatggctgcaggtgggagcatatggtggtttataaaaacgctgtcgggctttgtttccttctttagctgccgtgtctacttctgaagtctgggaagtgccaagccacgcggcctcaagggagctggctggtgtttgagctgtggcagaagcacctggggctccagggagcaggctgggaactgcaggaccttgctcagccaggagcacttccccctccttgaggcaggaatactgaggtgcctccccacagatggagaaggtggagaggaggatgggcctcaggagcatctcaagccccagtagcaggagaaagaaagaaagagatgcctggttttcacagactggttcctgtggctgggatgactgcatccttttttttttttttttgagacggagtttttgcctttgtcgcccaggctggagtgcaatggcgtgatctcggctcaccgcaacctccgcctcccggattcaagcaattctcctgcctcagcctcccgagtagctgggattacaggcacgcacctccacgtccggctaattttgtatttttagtggagacggggtttctccatgtcggtcaggctggtctcgaactcccgacctcaggtgatctgcccacctcggcctcccaaagtgctgggattacaggcatgagccaccgcgcttggccagactgcgtcctttttaagcgaacattttagggcctgggagtttgtcaagtaaggaagtctcaagcccaaagagcagcgtcctgaccatggtggtttcattacgagcccttctgctggctctcaggcagaagccccacagcaccgggaccattcatgaggtcactgcccagctcatgatgtccgtgaggctgtccttttggccagtagccgtgtgcagctgtgtggcacagatggcttcgttcatcctgatcaaggccccacctcagccacagcagtccccccaacctgtgttgtccaccctattattcatgtacctgccaggccctgctagatagcaccccgtggcattacataacacttcatgagtggctgtgtcttgtaattttggggacaggtttctctctttccctctcttttttttgtcaaaagcccagagactgacaaccagctgcagtgtctaagtgttcctcactgacagggtggggcctcaccacccctggagggagcagcgttggcagggagacagcctggcccagtgaccctgggcccaagccagcccctccagggctttcagggaagcgccatccattttcaaagatgtcaaacgtcacttcttcctgtagggcccgagtcctgcctcctatcagggccagatcatagaaggctattttctattctggggaacgattataacttaaatgattgttttaataaaaattctaagctggaaaataaaaaaaaaaaaaaaaaa

SEQ ID NO: 10 - protein, 595 aa, Homo sapiens
MAGAIASRMSFSSLKRKQPKTFTVRIVTMDAEMEFNCEMKWKGKDLFDLVCRTLGLRETWFFGLQYTIKDTVAWLKMDKKVLDHDVSKEEPVTFHFLAKFYPENAEEELVQEITQHLFFLQVKKQILDEKIYCPPEASVLLASYAVQAKYGDYDPSVHKRGFLAQEELLPKRVINLYQMTPEMWEERITAWYAEHRGRARDEAEMEYLKIAQDLEMYGVNYFAIRNKKGTELLLGVDALGLHIYDPENRLTPKISFPWNEIRNISYSDKEFTIKPLDKKIDVFKFNSSKLRVNKLILQLCIGNHDLFMRRRKADSLEVQQMKAQAREEKARKQMERQRLAREKQMREEAERTRDELERRLLQMKEEATMANEALMRSEETADLLAEKAQITEEEAKLLAQKAAEAEQEMQRIKATAIRTEEEKRLMEQKVLEAEVLALKMAEESERRAKEADQLKQDLQEAREAERRAKQKLLEIATKPTYPPMNPIPAPLPPDIPSFNLIGDSLSFDFKDTDMKRLSMEIEKEKVEYMEKSKHLQEQLNELKTEIEALKLKERETALDILHNENSDRGGSSKHNTIKKLTLQSAKSRVAFFEEL

SEQ ID NO: 11 - DNA, 5408 bp, Homo sapiens
gccgccgccagggaaaagaaagggaggaaggaaggaacaagaaaaggaaataaagagaaaggggaggcggggaaaggcaacgagctgtccggcctccgtcaagggagttggagggaaaaagttctcaggcgccgcaggtccgagtgcctcgcagcccctcccgaggcgcagccgccagaccagtggagccggggcgcagggcgggggcggaggcgccggggcgggggatgcggggccgcggcgcagccccccggccctgagagcgaggacagcgccgcccggcccgcagccgtcgccgcttctccacctcggcccgtggagccggggcgtccgggcgtagccctcgctcgcctgggtcagggggtgcgcgtcgggggaggcagaagccatggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggtgaggccacaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatagagccctcaggcagactgaattctaaatctgtgaaggatctaaggagacacatgcaccggaaatttccataagccagttgcagttttcaggctaatacagaaaaagatgaacaaacgtccagcaagatactttaatcctctattttgctcttccttgtccattgctgctgttaatgtattgctgacctctttcacagttggctctaaagaatcaaaagaaaaaaactttttatttcttttgctattaaaactactgttcattttgggggctgggggaagtgagcctgtttggatgatggatgccattccttttgcccagttaaatgttcaccaatcattttaactaaatactcagacttagaagtcagatgcttcatgtcacagcatttagtttgttcaacagttgtttcttcagcttcctttgtccagtggaaaaacatgatttactggtctgacaagccaaaaatgttatatctgatattaaatacttaatgctgatttgaagagatagctgaaaccaaggctgaagactgttttactttcagtattttcttttcctcctagtgctatcattagtcacataatgaccttgattttattttaggagcttataaggcatgagacaatttccatataaatatattaattattgccacatactctaatatagattttggtggataattttgtgggtgtgcattttgttctgttttgttgggttttttgttttttttgtttttggcagggtcggtgggggggttggttggttggttggttttgtcggaacctaggcaaatgaccatattagtgaatctgttaatagttgtagcttgggatggttattgtagttgttttggtaaaatcttcatttcctggttttttttaccaccttatttaaatctcgattatctgctctctcttttatatacatacacacacccaaacataacatttataatagtgtggtagtggaatgtatccttttttaggtttccctgctttccagttaatttttaaaatggtagcgctttgtatgcatttagaatacatgactagtagtttatatttcactggtagtttaaatctggttggggcagtctgcagatgtttgaagtagtttagtgttctagaaagagctattactgtggatagtgcctaggggagtgctccacgccctctgggcatacggtagatattatctgatgaattggaaaggagcaaaccagaaatggctttattttctcccttggactaatttttaagtctcgattggaattcagtgagtaggttcataatgtgcatgacagaaataagctttatagtggtttaccttcatttagctttggaagttttctttgccttagttttggaagtaaattctagtttgtagttctcatttgtaatgaacacattaacgactagattaaaatattgccttcaagattgttcttacttacaagacttgctcctacttctatgctgaaaattgaccctggatagaatactataaggttttgagttagctggaaaagtgatcagattaataaatgtatattggtagttgaatttagcaaagaaatagagataatcatgattatacctttatttttacaggaagagatgatgtaactagagtatgtgtctacaggagtaataatggtttccaaagagtattttttaaaggaacaaaacgagcatgaattaactcttcaatataagctatgaagtaatagttggttgtgaattaaagtggcaccagctagcacctctgtgttttaagggtctttcaatgtttctagaataagcccttattttcaagggttcataacaggcataaaatctcttctcctggcaaaagctgctatgaaaagcctcagcttgggaagatagatttttttccccccaattacaaaatctaagtattttggcccttcaatttggaggagggcaaaagttggaagtaagaagttttattttaagtactttcagtgctcaaaaaaatgcaatcactgtgttgtatataatagttcataggttgatcactcataataattgactctaaggcttttattaagaaaacagcagaaagattaaatcttgaattaagtctggggggaaatggccactgcagatggagttttagagtagtaatgaaattctacctagaatgcaaaattgggtatatgaattacatagcatgttgttgggattttttttaatgtgcagaagatcaaagctacttggaaggagtgcctataatttgccagtagccacagattaagattatatcttatatatcagcagattagctttagcttagggggagggtgggaaagtttggggggggggttgtgaagatttagggggaccttgatagagaactttataaacttctttctctttaataaagacttgtcttacaccgtgctgccattaaaggcagctgttctagagtttcagtcacctaagtacacccacaaaacaatatgaatatggagatcttcctttacccctcaactttaatttgcccagttatacctcagtgttgtagcagtactgtgatacctggcacagtgctttgatcttacgatgccctctgtactgacctgaaggagacctaagagtcctttccctttttgagtttgaatcatagccttgatgtggtctcttgttttatgtccttgttcctaatgtaaaagtgcttaactgcttcttggttgtattgggtagcattgggataagattttaactgggtattcttgaattgcttttacaataaaccaattttataatctttaaatttatcaactttttacatttgtgttattttcagtcagggcttcttagatctacttatggttgatggagcacattgatttggagtttcagatcttccaaagcactatttgttgtaataacttttctaaatgtagtgcctttaaaggaaaaatgaacacagggaagtgactttgctacaaataatgttgctgtgttaagtattcatattaaatacatgccttctatatggaacatggcagaaagactgaaaaataacagtaattaattgtgtaattcagaattcataccaatcagtgttgaaactcaaacattgcaaaagtgggtggcaatattcagtgcttaacacttttctagcgttggtacatctgagaaatgagtgctcaggtggattttatcctcgcaagcatgttgttataagaattgtgggtgtgcctatcataacaattgttttctgtatcttgaaaaagtattctccacattttaaatgttttatattagagaattctttaatgcacacttgtcaaatatatatatatagtaccaatgttacctttttattttttgttttagatgtaagagcatgctcatatgttaggtacttacataaattgttacattattttttcttatgtaatacctttttgtttgtttatgtggttcaaatatattctttccttaaactcttaaaaaaaaaa

SEQ ID NO: 12 - protein, 508 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQVRPQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL